(19) United States
(12) Patent Application Publication
(10) Pub. No.: US 2013/0184318 A1
(43) Pub. Date: Jul. 18, 2013

(54) MODULATING COMPLIANCE OF TRABECULAR MESHWORK

(71) Applicants: Paul Russell, Davis, CA (US); Christopher J. Murphy, Davis, CA (US)

(72) Inventors: Paul Russell, Davis, CA (US); Christopher J. Murphy, Davis, CA (US)

(73) Assignee: THE REGENTS OF THE UNIVERSITY OF CALIFORNIA, Oakland, CA (US)

(21) Appl. No.: 13/643,543

(22) PCT Filed: Apr. 28, 2011
(86) PCT No.: PCT/US2011/034259

§ 371 Date: Dec. 15, 2012

Related U.S. Application Data
(60) Provisional application No. 61/329,885, filed on Apr. 30, 2010.

(51) Int. Cl. C12Q 1/02

(52) U.S. Cl. CPC C12Q 1/025
USPC 514/369; 435/29; 548/187; 435/375; 435/6.12; 435/7.1

(57) ABSTRACT

The present invention provides methods of determining the effect of test agents on the compliance of trabecular meshwork (TM) tissue, at the level of the extracellular matrix (ECM) and/or cells within the TM. Agents that modulate (e.g., increase or decrease) compliance of the TM tissue find use. Agents that increase the compliance of ECM and TM cells within the TM find use in the treatment and prevention of eye disorders characterized by high intraocular pressure, e.g., glaucoma.

### CROSS-REFERENCE TO RELATED APPLICATIONS

This application claims the benefit of U.S. Provisional Application No. 61/329,885, filed on Apr. 30, 2010, the entire disclosure of which is hereby incorporated herein by reference.

### STATEMENT AS TO RIGHTS TO INVENTIONS MADE UNDER FEDERALLY SPONSORED RESEARCH AND DEVELOPMENT

This invention was made with government support under Grant Nos. HL079012, R01EY019475 and 5R01EY016134, awarded by the National Institutes of Health (NIH). The government has certain rights in the invention.

### FIELD OF THE INVENTION

The present invention provides methods of determining the effect of test agents on the compliance of trabecular meshwork (TM) tissue, at the level of the extracellular matrix (ECM) and/or cells within the TM. Agents that modulate (e.g., increase or decrease) compliance of the TM tissue find use. Agents that increase the compliance (i.e., decrease the stiffness) of ECM and TM cells within the TM find use in the treatment and prevention of eye disorders characterized by high intraocular pressure, e.g., glaucoma.

### BACKGROUND OF THE INVENTION

### Glaucoma and Trabecular Meshwork

Glaucoma is a family of irreversible blinding diseases that are predicted to affect 79.6 million people worldwide by the year 2020¹. Primary open angle glaucoma (POAG) is characterized by an increased resistance to aqueous humor outflow. To date, the only rigorously validated treatment for glaucoma is therapeutically lowering the intraocular pressure (IOP)^{2,3,4}. Many studies have sought to determine the principal site responsible for outflow resistance in the eye⁵. Johnstone and Grant demonstrated that increasing IOP causes the trabecular meshwork to dilate through expansion of the spaces in the inner meshwork⁶. Their work implicates the juxtacanalicular region (JCT) of the meshwork at Schlemm's canal as the principal site of outflow resistance in this tissue⁷. A schematic diagram of the anatomy of the TM⁸ is shown in FIG. 2. A secondary outflow site for aqueous humor is the uveo-scleral pathway also named the unconventional outflow pathway. While this pathway has been the target for some therapeutic interventions in glaucoma, it is not the principal outflow pathway in adult humans. Alterations in other tissues may also be occurring throughout the eye during the progression of glaucoma.

Alterations in basement membranes have been implicated in many diseases^{9,10,11}. The analysis of the basement membrane components of the JCT cells revealed a membrane rich in collagen IV as well as an elastic network^{12,13}. Studies have correlated the outflow resistance with several key components of the extracellular matrix in the JCT^{14,15}. Meshwork cells express many glycoaminoglycans (GAGs) including hyaluronic acid and chondroitin sulfate¹⁶. The composition of the GAGs changes in glaucoma with increasing levels of chondroitin sulfate compared to hyaluronic acid¹⁷. Chondroitin sulfate glycoprotein 2, thought to play a role in regulating IOP, has multiple splice variants in HTM cells¹⁸. Changes in the composition of these spliced forms are related to reduced outflow. The excess synthesis of laminin and collagen IV associated with the glaucomatous meshwork, in addition to decreased remodeling of the HTM extracellular matrix, are thought to contribute to the decrease in outflow facility^{19,20}. Significant levels of cross-linked fibronectin, an increased level of the cross-linking enzyme, tissue transglutaminase and protein adducts caused by lipid oxidation have been found in the meshwork from glaucomatous eyes. These could also contribute to an increase in outflow resistance²¹.

### BRIEF SUMMARY OF THE INVENTION

The present invention provides methods of identifying agents that modulate (e.g. increase or decrease) the compliance of trabecular meshwork (TM) tissue, including individual cells and/or the extracellular matrix (ECM) of the TM, by contacting the TM with a test agent and determining the changes in the compliance of the TM. The changes in compliance can be determined on the same cell or tissue before and after contacting with the agent. Alternatively, changes in compliance can be determined by comparing the compliance of a cell or ECM of the TM that is contacted with a test agent with the compliance of a cell or ECM of the TM that is not contacted with the test agent. The identified agents find use in modulating (e.g., increasing or decreasing) the compliance of trabecular meshwork tissue. Agents that increase the compliance (i.e., decrease the stiffness or elastic modulus) of the TM, including agents that disrupt the cytoskeleton of a TM cell and/or remodel the ECM of the TM, find use in decreasing intraocular pressure, promoting outflow of aqueous humor through the TM, and therefore reducing the severity of glaucoma in patients in need thereof.

Accordingly, in one aspect, the invention provides methods of identifying an agent that modulates the intrinsic compliance of a trabecular meshwork (TM) cell. In some embodiments, the methods comprise:

a) contacting the agent to a test TM cell cultured in vitro;

b) determining the compliance of the test TM cell. In some embodiments, the test cell is compared to an untreated control cell. In such cases, a Young's modulus of the test TM cell that is different from the Young's modulus of a control TM cell not contacted with the agent indicates that the agent modulates the compliance of the TM cell. In some embodiments, the compliance of the same test cell is measured before and after contacting the cell with the test agent. In such cases, a Young's modulus of the TM cell after exposure to the test agent that is different from the Young's modulus of the same TM cell prior to exposure to the test agent indicates that the agent modulates the compliance of the TM cell.

With respect to the methods of identifying agents that modulate the compliance of a TM cell, in some embodiments, the intrinsic compliance of a single TM cell or a region of a TM cell is measured.

In some embodiments, the TM cell is cultured on a surface having fabricated topographical features. In some embodiments, the TM cell is cultured on a surface having different or varied values of compliance. In some embodiments, the TM cell is cultured on a surface with a Young's modulus value in the range of about 3 kPa to about 100 kPa. For example, the surface may mimic compliant conditions of normal (i.e., homeomimetic) HTM tissue. In some embodiments, the TM cell is cultured on a surface with a Young's modulus value that is under 10 kPa, for example, in the range of about 3 kPa to about 10 kPa, for example, about 3 kPa, 4 kPa, 5 kPa, 6 kPa, 7 kPa, 8 kPa, 9 kPa or 10 kPa. In various embodiments, the surface may mimic the compliant conditions of glaucomatous (i.e., pathomimetic) HTM tissue. In some embodiments, the TM cell is cultured on a surface with a Young's modulus value that is greater than 10 kPa, for example, in the range of about 10 kPa to about 100 kPa, for example, about 10 kPa, 20 kPa, 30 kPa, 40 kPa, 50 kPa, 60 kPa, 70 kPa, 80 kPa, 90 kPa or 100 kPa. In some embodiments, the TM cell is cultured on a hydrogel. Illustrative hydrogels that find use as culture surfaces for TM cells include without limitation those based upon poly(ethylene glycol)diacrylate (PEGDA), poly (2-hydroxyethylmethacrylate) (PHEMA), collagen I, collagen IV, elastin, hyaluronic acid (HA), polyacrylamide and hybrids of these components.

In some embodiments, the TM cell is from a glaucomatous subject. In some embodiments, the TM cell is from a non-glaucomatous subject. In some embodiments, the subject is human. In some embodiments, the TM cell is from the juxtacanalicular region (JCT).

In various embodiments, the subject is a mammal. For example, in various embodiments, the subject is a human, dog, cat, cow or pig.

In some embodiments, the intrinsic compliance is measured by atomic force microscopy (ATM).

In some embodiments, the agent decreases the intrinsic compliance (i.e., increases stiffness or elastic modulus) of the TM cell. In some embodiments, the agent increases the intrinsic compliance (i.e., decreases stiffness or elastic modulus) of the TM cell. For example, the agent may disrupt the cytoskeleton of the cell, e.g., by disrupting intermediate filaments, microtubules and/or actin polymerization. In some embodiments, the agent increases or decreases gelation of the cytosol, independent of altering cytoskeletal dynamics. In some embodiments, the agent increase or decreases compliance of the lipid bilayer of the TM cell.

In a related aspect, the invention provides methods of identifying an agent that modulates the intrinsic compliance of an extracellular matrix (ECM) within a trabecular meshwork (TM). In some embodiments, the methods comprise:

a) contacting the agent to a test ECM of TM tissue cultured in vitro;

b) determining the compliance of the test ECM. In some embodiments, the test ECM is compared to an untreated control ECM. In such cases, a Young's modulus of the test ECM that is different from the Young's modulus of a control ECM not contacted with the agent indicates that the agent modulates the compliance of the TM cell. In some embodiments, the compliance of the same test ECM is measured before and after contacting the ECM with the test agent. In such cases, a Young's modulus of the ECM after exposure to the test agent that is different from the Young's modulus of the same ECM prior to exposure to the test agent indicates that the agent modulates the compliance of the ECM.

With respect to the methods for identifying agents that modulate the ECM of TM tissue, in some embodiments, the TM tissue is from a glaucomatous subject. In some embodiments, the TM tissue is from a non glaucomatous subject. In some embodiments, the TM tissue is from the juxtacanalicular region (JCT).

In some embodiments, the TM tissue prior to exposure to the test agent has a Young's modulus value that is under 10 kPa, for example, in the range of about 1.5 kPa to about 10 kPa, for example, about 1.5 kPa, 2 kPa, 2.5 kPa, 3 kPa, 3.5 kPa, 4 kPa, 4.5 kPa, 5 kPa, 5.5 kPa, 6 kPa, 6.5 kPa, 7 kPa, 8 kPa, 9 kPa or 10 kPa. In some embodiments, the TM tissue prior to exposure to the test agent has a Young's modulus value that is greater than 10 kPa, for example, in the range of about 10 to 140 kPa, or about 50 to 140 kPa, or about 70 to 140 kPa, for example, about 10 kPa, 20 kPa, 30 kPa, 40 kPa, 50 kPa, 60 kPa, 70 kPa, 80 kPa, 90 kPa, 100 kPa, 110 kPa, 120 kPa, 130 kPa or 140 kPa.

In some embodiments, the agent increases the intrinsic compliance (i.e., decreases stiffness or elastic modulus) of the ECM. In some embodiments, the agent decreases the intrinsic compliance (i.e., increases stiffness or elastic modulus) of the ECM.

In some embodiments, the intrinsic compliance is measured by a method selected from the group consisting of indenting, pulling, compressing, shearing, bending, buckling, wave propagation, and drilling. In some embodiments, the intrinsic compliance is measured by atomic force microscopy (ATM).

With respect to agents that increase the compliance of the ECM of the TM, in some embodiments, the agent increases expression or activity of matricellular proteins, for example, increases the expression of fibronectin, myocilin (NM_{—}000261.1→NP_{—}000252.1) and/or secreted protein, acidic, cysteine-rich (osteonectin or SPARC) (NM_{—}003118.2→NP_{—}003109.1).

In some embodiments, the agent modulates (e.g., increases or decreases) expression or activity of enzymes that degrade, erode or alter the ECM, including but not limited to matrix metalloproteinases (MMPs), disintegrin and metalloproteinases (ADAMs) and disintegrin and metalloproteinase with thrombospondin motifs (ADAM-TSs). Agents that increase compliance (i.e., decrease stiffness or elastic modulus) increase the expression or activity of enzymes that degrade, erode or alter the ECM. Exemplary MMPs include without limitation MMP-1 (e.g., NM_{—}001145938.1→NP_{—}001139410.1 (interstitial collagenase isoform 2); NM_{—}002421.3→NP_{—}002412.1 (interstitial collagenase isoform 1), MMP-2 (NM_{—}001127891.1→NP_{—}001121363.1 (72 kDa type IV collagenase isoform b); NM_{—}004530.4→NP_{—}004521.1 (72 kDa type IV collagenase isoform a), MMP-3 (NM_{—}002422.3→NP_{—}002413.1 (stromelysin-1)), MMP-9 (NM_{—}004994.2→NP_{—}004985.2), MMP-11 (NM_{—}005940.3→NP_{—}005931.2 (stromelysin-3)), MMP-12 (NM_{—}002426.4→NP_{—}002417.2), MMP-14 (NM_{—}004995.2→NP_{—}004986.1 (membrane-type MMP or MT1-MMP)), MMP-15 (NM_{—}002428.2→NP_{—}002419.1), MMP-16 (NM_{—}005941.4→NP_{—}005932.2 (matrix metalloproteinase-16 isoform 1); NM_{—}022564.3→NP_{—}072086.2 (matrix metalloproteinase-16 isoform 2)), MMP-17 (NM_{—}016155.4→NP_{—}057239.4) and MMP-19 (NM_{—}002429.4→NP_{—}002420.1 (matrix metalloproteinase-19 isoform rasi-1)).

Exemplary ADAMs include without limitation ADAM metallopeptidase domain 2 (ADAM2; NM_{—}001464.3→NP_{—}001455.3); ADAM metallopeptidase domain 7 (ADAM7; NM_{—}003817.2→NP_{—}003808.2); ADAM metallopeptidase domain 8 (ADAM8; NM_{—}001109.4→NP_{—}001100.3 (isoform 1); NM_{—}001164489.1→NP_{—}001157961.1 (isoform 2); NM_{—}001164490.1→NP_{—}001157962.1 (isoform 3)); ADAM metallopeptidase domain 9 (meltrin gamma or ADAM9; NM_{—}003816.2→NP_{—}003807.1); ADAM metallopeptidase domain 10 (ADAM10; NM_{—}001110.2→NP_{—}001101.1); ADAM metallopeptidase domain 11 (ADAM11; NM_{—}002390.4→NP_{—}002381.2); ADAM metallopeptidase domain 12 (ADAM12; NM_{—}003474.4→NP_{—}003465.3 (isoform 1); NM_{—}021641.3→NP_{—}067673.2 (isoform 2)); ADAM metallopeptidase domain 15 (ADAM15; NM_{—}003815.3→NP_{—}003806.3 (isoform 1); NM_{—}207191.1→NP_{—}997074.1 (isoform 2); NM_{—}207194.1→NP_{—}997077.1 (isoform 3); NM_{—}207195.1→NP_{—}997078.1 (isoform 4); NM_{—}207196.1→NP_{—}997079.1 (isoform 5); NM_{—}207197.1→NP_{—}997080.1 (isoform 6)); ADAM metallopeptidase domain 17 (ADAM17; NM_{—}003183.4→NP_{—}003174.3); ADAM metallopeptidase domain 18 (ADAM18; NM_{—}014237.1→NP_{—}055052.1); ADAM metallopeptidase domain 19 (meltrin beta or ADAM19; NM_{—}033274.2→NP_{—}150377.1); ADAM metallopeptidase domain 20 (ADAM20; NM_{—}003814.4→NP_{—}003805.3); ADAM metallopeptidase domain 21 (ADAM21; NM_{—}003813.2→NP_{—}003804.1); ADAM metallopeptidase domain 22 (ADAM22; NM_{—}004194.2→NP_{—}004185.1 (isoform 4); NM_{—}016351.3→NP_{—}057435.2 (isoform 3); NM_{—}021721.2→NP_{—}068367.1 (isoform 5); NM_{—}021722.3→NP_{—}068368.2 (isoform 2); NM_{—}021723.2→NP_{—}068369.1 (isoform 1)); ADAM metallopeptidase domain 23 (ADAM23; NM_{—}003812.2→NP_{—}003803.1); ADAM metallopeptidase domain 28 (ADAM28; NM_{—}014265.4→NP_{—}055080.2 (isoform 1); NM_{—}021777.3→NP_{—}068547.2 (isoform 3)); ADAM metallopeptidase domain 29 (ADAM29; NM_{—}001130703.1→NP_{—}001124175.1 (variant 2); NM_{—}001130704.1→NP_{—}001124176.1 (variant 3); NM_{—}001130705.1→NP_{—}001124177.1 (variant 4); NM_{—}014269.4→NP_{—}055084.3 (variant 1)); ADAM metallopeptidase domain 30 (ADAM30; NM_{—}021794.2→NP_{—}068566.2); ADAM metallopeptidase domain 33 (ADAM33; NM_{—}025220.2→NP_{—}079496.1 (isoform alpha); NM_{—}153202.1→NP_{—}694882.1 (isoform beta)).

Exemplary ADAM-TSs include without limitation ADAM-TS1 (NM_{—}006988.3→NP_{—}008919.3), ADAM-TS4 (NM_{—}005099.4→NP_{—}005090.3), and ADAM-TS5 (NM_{—}007038.3→NP_{—}008969.2).

The agent can be a substrate analog for an MMP, ADAM or ADAM-TS enzyme, wherein the analog modulates (e.g., increases or decreases) the catalytic activity of the enzyme. Agents that increase compliance (i.e., decrease stiffness or elastic modulus) increase the catalytic activity of an MMP, ADAM or ADAM-TS enzyme. Agents that decrease compliance (i.e., increase stiffness or elastic modulus) decrease the catalytic activity of an MMP, ADAM or ADAM-TS enzyme. In other embodiment, the agent can modulate effector molecules that inhibit MMPs, ADAMs, ADAM-TSs and other enzymes that degrade or modify the extracellular matrix. Such an agent is exemplified by but is not limited to inhibitors of Tissue Inhibitors of Matrix Metalloproteinases (TIMPs) that inhibit enzyme activity. Exemplary TIMPs include without limitation TIMP1 (NM_{—}003254.2→NP_{—}003245.1), TIMP2 (NM_{—}003255.4→NP_{—}003246.1), TIMP3 (NM_{—}000362.4→NP_{—}000353.1) and TIMP4 (NM_{—}003256.2→NP_{—}003247.1). In one embodiment, the agent is an inhibitory nucleic acid (e.g., an siRNA) that inhibits the expression of one or more TIMPs and thus augments the degradative action of the ECM by native enzymes modulated by TIMPs. In another embodiment, the agent enhances promoter activation of a MMP, ADAM or ADAM-TS gene, thereby increasing endogenous activity of the enzyme. See, e.g., Brew, et al., (2000). Biochim Biophys Acta 1477 (1-2): 267-83; Lee, et al., J Biol Chem. (2005) 280(16):15967-75; Seals and Courtneidge (2003) Genes & Dev. 17:7-30.

The expression and activity of MMPs can be modulated by agents including prostaglandins, COX-2 inhibitors and tetracyclines. Generally, prostaglandins and COX-2 inhibitors up-regulate MMP activity. However, selective COX-2 inhibitors can inhibit expression of MMP9 and tetracyclines decrease MMP activity. Prostaglandin F(2 alpha) (PGF2alpha) inhibits expression of TIMP1, resulting in increased expression of MMPs See, e.g., Ricke, et al., (2002) Biology of Reproduction 66(3):685-691; Ito, et al., (2004) Cancer Research 64:7439-7446 and Steenport, et al., J Immunol. (2009) 183(12):8119-27.

In some embodiments, the agent decreases cross-linking of the ECM. For example, the agent may decrease the cross-linking of one or more ECM components, including but not limited to fibronectin or a collagen, for example, collagen I, collagen III, collagen IV and collagen VI. In some embodiments, the agent decreases or inhibits the expression and/or activity of a transglutaminase.

In some embodiments, the agent increases the ratio of glycosaminoglycans hyaluronic acid (HA) to chondroitin SO₄, or increase the hyaluronic acid. For example, the agent may decrease the expression of a versican isoform, a chondroitin sulfate proteoglycan.

In a related aspect, the invention provides methods of increasing the compliance (i.e., decreasing stiffness or elastic modulus) of a trabecular meshwork (TM), comprising contacting the TM with an agent that increases the compliance of a TM cell, the agent being identified by the methods described herein.

In another aspect, the invention provides agents for increasing the compliance (i.e., decreasing stiffness or elastic modulus) of a trabecular meshwork (TM), the agent being identified by the methods described herein. For example, the invention further contemplates agents that modulate the intrinsic compliance of a trabecular meshwork (TM) cell and/or agents that modulate the intrinsic compliance of an extracellular matrix (ECM) within a trabecular meshwork (TM), wherein the agents are identified by the screening methods described herein.

In some embodiments, the TM is contacted with the agent in vivo. In some embodiments, the TM is contacted with the agent in vitro.

In a related aspect, the invention provides methods of reducing intraocular pressure in a subject in need thereof, comprising administering to the subject an effective amount of an agent that increases the compliance (i.e., decreases the stiffness or elastic modulus) of a TM cell and/or an agent that increases the compliance (i.e., decreases the stiffness or elastic modulus) of an ECM in the TM, wherein the outflow of aqueous humor through the TM is increased, thereby reducing intraocular pressure in the subject. The agents may be identified according to the methods described herein. In various embodiments, a combination of agents is administered, wherein the first agent increases the compliance (i.e., decreases the stiffness or elastic modulus) of a TM cell and the second agent increases the compliance (i.e., decreases the stiffness or elastic modulus) of an ECM in the TM.

In a further aspect, the invention provides methods of reducing the severity of glaucoma in a subject in need thereof, comprising administering to the subject an effective amount of an agent that increases the compliance (i.e., decreases the stiffness or elastic modulus) of the TM cell and/or an agent that increases the compliance (i.e., decreases the stiffness or elastic modulus) of the ECM in the TM, wherein the outflow of aqueous humor through the TM is increased, thereby reducing the severity of glaucoma in the subject. The agent may be identified according to the methods described herein. In various embodiments, a combination of agents is administered, wherein the first agent increases the compliance (i.e., decreases the stiffness or elastic modulus) of a TM cell and the second agent increases the compliance (i.e., decreases the stiffness or elastic modulus) of an ECM in the TM.

With respect to the embodiments of the methods for treatment and/or prevention of glaucoma or reducing intraocular pressure, known agents that disrupt the cytoskeleton of a cell find use. Therefore one or more agents that disrupt intermediate filaments, microtubules, or actin polymerization can be administered. Exemplary agents that disrupt intermediate filaments include without limitation acrylamide, calpain-1 (NM_{—}005186.2→NP_{—}005177.2), calpain-2 (NM_{—}001146068.1→NP_{—}001139540.1 (calpain-2 catalytic subunit isoform 2); NM_{—}001748.4→NP_{—}001739.2 (calpain-2 catalytic subunit isoform 1; NM_{—}032330.1→NP_{—}115706.1 (calpain small subunit 2)), rho kinase inhibitors, blebbistatin, caldesmon (NM_{—}033138.3→NP_{—}149129.2 (isoform 1); NM_{—}033139.3→NP_{—}149130.1; NM_{—}004342.6→NP_{—}004333.1 (isoform 2); NM_{—}033157.3→NP_{—}149347.2 (isoform 3); NM_{—}033139.3→NP_{—}149130.1 (isoform 4); NM_{—}033140.3→NP_{—}149131.1 (isoform 5)), and inhibitory RNA (RNAi) that inhibits expression of proteins involved in formation and maintenance of intermediate filaments. Exemplary agents that disrupt microtubules include without limitation colchicine, colecemid, vinca alkaloids (e.g., vinblastine, vincristine, vinorelbine, vindesine), podophyllotoxin, capecotobine, dolastatin 15, nocodazole, tryprostatin A, rhizoxin, vinflunine, epothilones, ixabepilone, methyl benzimidazol-2-yl-carbamate, estramustine sodium phosphate, taxanes (e.g., paclitaxel, docetaxil, colchitaxel), and indibulin. Exemplary agents that disrupt actin polymerization include without limitation cytochalasin B, cytochalasin D, latrunculin A, latrunculin B, migrastatin, E47 transcription factor for semaphorin 3F, semaphorin 3F (NM_{—}004186.3→NP_{—}004177.3), actin depolymerizing factor (NM_{—}001011546.1→NP_{—}001011546.1 (destrin isoform b); NM_{—}006870.3→NP_{—}006861.1 (destrin isoform a)), cucurbitane-type tritepenes B&E, gelsolin (NM_{—}001747.2→NP_{—}001738.2 (macrophage-capping protein)), olivetoric acid, chivosazole A, chivosazole F, clostridium perfringens iota, clostridium botulinum C2, and desmethoxymajusculamide C.

In some embodiments, the agent administered increases the compliance (i.e., decreases the stiffness or elastic modulus) of the ECM within the TM tissue. Exemplary agents are described above and herein.

In some embodiments, the agent or agents are administered intraocularly. In some embodiments, the agent or agents are administered topically. In some embodiments, the agent or agents are administered directly in and/or around the TM tissue. In some embodiments, the agent or agents are administered systemically, e.g., orally. In some embodiments, the subject is glaucomatous.

### DEFINITIONS

The term “compliance” refers to the property of a body or substance of yielding to an applied force or of allowing a change to be made in its shape; also, the degree of yielding, measured by the displacement produced by a unit change in the force. Compliance can be quantified by elastic modulus or Young's modulus, in units of kPa.

The terms “Young's modulus” or “elastic modulus” interchangeably refer to the ratio of the stress acting on a substance to strain produced.

The term “specifically inhibit” refers to the ability of an agent or ligand to inhibit the expression or the biological function of a target protein. Specific inhibition typically results in at least about a 2-fold inhibition over background, preferably greater than about 10-fold and most preferably greater than 100-fold inhibition of expression (e.g., transcription or translation) of the target protein or measured biological function, for example, by comparing treated and untreated cells, or a cell population before and after treatment. In some embodiments, the expression or biological function of the target protein is completely inhibited. Typically, specific inhibition is a statistically meaningful reduction in expression or biological function (e.g., p≦0.05) using an appropriate statistical test.

The terms “bind(s) specifically” or “specifically bind(s)” or “attached” or “attaching” refers to the preferential association of an agent or ligand, in whole or part, with a target epitope that binds or competes with another agent or ligand for binding to the target epitope expressed on a cell or tissue. It is, of course, recognized that a certain degree of non-specific interaction may occur between an antibody and a non-target epitope. Nevertheless, specific binding, can be distinguished as mediated through specific recognition of the target epitope. Typically specific binding results in a much stronger association between the delivered molecule and an entity (e.g., an assay well or a cell) bearing the target epitope than between the bound antibody and an entity (e.g., an assay well or a cell) lacking the target epitope. Specific binding typically results in at least about a 2-fold increase over background, preferably greater than about 10-fold and most preferably greater than 100-fold increase in amount of bound agent or ligand (per unit time) to a cell or tissue bearing the target epitope as compared to a cell or tissue lacking the target epitope. Specific binding between two entities generally means an affinity of at least 10⁶ M⁻¹. Affinities greater than 10⁸ M⁻¹ or greater are preferred. Specific binding can be determined for nucleic acid as well as protein agents and ligands. Specific binding for nucleic acid agents can be determined using any assay known in the art, including but not limited to northern blots, gel shift assays and in situ hybridization. Specific binding for protein agents and ligands can be determined using any binding assay known in the art, including but not limited to gel electrophoresis, Western blot, ELISA, flow cytometry, and immunohistochemistry.

The term “agent” as used herein refers to polypeptides (e.g., ligands, antibodies), peptidomimetics, nucleic acids, small organic compounds, and the like.

The term “inhibitory nucleic acid” refers to a single-stranded nucleic acid that specifically binds or hybridizes to a complementary nucleic acid to inhibit or decrease gene expression of the complementary nucleic acid. Exemplary inhibitory nucleic acids suitable for use with the present invention include small interfering RNA (“siRNA” or “RNAi”), short hairpin RNA (shRNA), micro RNA (“miRNA”), antisense, ribozymes, and the like.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

The term “ligand” as used herein refers to a molecule that binds specifically to another molecule. A ligand can be a protein, a nucleic acid, or a chemical compound. Ligands that are proteins can include antibody, and non-antibody specific binding agents, for example, those described herein.

The term “antibody” refers to an immunoglobulin molecule obtained by in vitro or in vivo generation of the humoral response, and includes both polyclonal and monoclonal antibodies. The term also includes genetically engineered forms such as chimeric antibodies (e.g., humanized murine antibodies), heteroconjugate antibodies (e.g., bispecific antibodies), and recombinant single chain Fv fragments (scFv). The term “antibody” also includes antigen binding forms of antibody fragments (e.g., Fab, F(ab)₂, V_{H}-V_{L} Fab fragments).

“Topical application” to the eye refers to the administration of an agent to the eye by applying the agent to the eyelids or to the conjunctival sac in aqueous or viscous solutions or suspensions, in ointments, as fine powders, on cotton pledgets, by drug-impregnated contact lenses, by injection into the eye, by mechanical pumps, or by membrane release systems.

The term “therapeutically effective amount” refers to an amount of the compound being administered sufficient to prevent or decrease the development of one or more of the symptoms of the disease, condition or disorder being treated. Preferably, a therapeutically effective amount achieves efficacy with minimal or no undesirable side effects.

The term “co-administer” refers to the simultaneous presence of two active agents in the blood of an individual. Active agents that are co-administered can be concurrently or sequentially delivered.

As used herein, the terms “treating” and “treatment” refer to delaying the onset of, retarding or reversing the progress of, or alleviating or preventing either the disease or condition to which the term applies, or one or more symptoms of such disease or condition.

The terms “subject,” “patient,” or “individual” interchangeably refer to a mammal, in particular, a human or a non-human primate. In some embodiments, the mammal is a domesticated mammal (e.g., canine or feline), an agricultural mammal (e.g., porcine, ovine, bovine, equine) or laboratory mammal (e.g., murine, rattus, lagomorpha, hamster).

The terms “identical” or percent “identity,” in the context of two or more nucleic acids or polypeptide sequences, refer to two or more sequences or subsequences that are the same. Sequences are “substantially identical” if they have a specified percentage of amino acid residues or nucleotides that are the same (i.e., at least 60% identity, optionally at least 65%, 70%, 75%, 80%, 85%, 90%, 95%, 98%, or 99% identity over a specified region (or the whole reference sequence when not specified)), when compared and aligned for maximum correspondence over a comparison window, or designated region as measured using one of the following sequence comparison algorithms or by manual alignment and visual inspection. The present invention provides for promoters that are substantially identical to any of the GenBank accession numbers described herein. Optionally, the identity exists over a region that is at least about 50 nucleotides or amino acids in length, or more preferably over a region that is 100 to 500 or 1000 or more nucleotides or amino acids in length, or over the full-length of the sequence.

The term “similarity,” or “percent similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Sequences having less than 100% similarity but that have at least one of the specified percentages are said to be “substantially similar.” Optionally, this identity exists over a region that is at least about 50 amino acids in length, or more preferably over a region that is at least about 100 to 500 or 1000 or more amino acids in length, or over the full-length of the sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Examples of an algorithm that is suitable for determining percent sequence identity and sequence similarity include the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### BRIEF DESCRIPTION OF THE DRAWINGS

FIG. 1 illustrates the interplay between the biophysical properties of the extracellular matrix (ECM), trabecular meshwork (TM) cells and outflow facility. Normal ECM of the meshwork is compliant and contains topographic features with which the trabecular meshwork cells can interact. In glaucoma, the matrix is stiff causing decreased compliance of the TM cells and reduced matrix degradation. With initiation or progression of glaucoma, dysregulation events can initiate a feedback loop that decreases compliance of the matrix thereby decreasing compliance of the TM cells and lowering outflow facility.

FIG. 2 illustrates a schematic diagram of the trabecular meshwork. The TM consists of two distinct parts. The inner meshwork is formed by lamellae completely covered by TM cells. Next to this region is the JCT that is formed by star-shaped TM cells and extracellular matrix. These TM cells are connected with one another and also to the inner wall cells of Schlemm's canal. Most of the aqueous humor passes through the TM.

FIG. 3 illustrates a confocal micrograph showing secreted protein, acidic, cysteine-rich (osteonectin or SPARC) localization in the TM. The TM was stained with antibodies for SPARC (green), phalloidin for actin (red), and DAPI for the nucleus (blue). TM=Trabecular Meshwork. SC=Schlemm's Canal. Note that the matricellular protein SPARC has extensive staining throughout the TM.

FIG. 4 illustrates that TM cells are modulated by topographic cues. The TM cells were cultured for 24 hours on a planar surface (A) and on a 400 nm pitch substrate (B). The TM cells are aligned and elongated on the topographically patterned substrate. The localization of the actin (phalloidin stain) appears unorganized on the planar surface compared to the topographically patterned substrate. Actin on the 400 nm pitch surface localized to ridges.

FIG. 5 illustrates the percent of TM cell alignment is modulated by topography. Cells were cultured for 24 hours prior to fixation and staining with phalloidin. Images were obtained and analyzed with Zeiss KS300 software. Cells between 0-10° of the patterned ridges were defined as aligned. On the planar surface, the cells were as equally likely to be aligned or perpendicular to an axis. On the 400 to 4000 nm pitch (400 to 4000) surfaces, the cells aligned in the direction of the grooves and ridges. The number of cells aligned above 400 nm pitch was between 70% to 90% depending on the donor TM.

FIG. 6 illustrates that TM cells cultured on substrates having low compliance have greater and more defined numbers of stress fibers. TM cells were cultured on (A) glass (50 GPa) or (B) polyacrylamide (3 kPa) and stained with phalloidin to reveal the actin cytoskeleton. Note the large numbers of stress fibers in the cells on the glass and the rounder shape of the cells on the gel. The alteration in cytoskeletal dynamics resulting from interaction with substrates of differing compliance ultimately results in modulation of the intrinsic compliance of the TM cell. Cells on less compliant substrates have lower intrinsic compliance.

FIG. 7 illustrates the compliance of glaucomatous tissue is decreased compared to normal TM. Atomic force microscopy (AFM) force curve from a normal 78 y.o. TM with a modulus=6.5 kPa (black) and a glaucomatous 92 y.o. with a modulus=75 kPa (blue). A steeper slope is indicative of a more rigid tissue. Force curves were taken with a 1 mm radius borosilicate sphere attached to a cantilever with a nominal spring constant of 0.32 N/m. The cantilever frequency was 8-9 kHz with an applied voltage of 500 mV. The RMS amplitude was 0.3-0.5 V with a scan rate of 0.3 Hz. All force curves were taken at a rate of 2 mm/sec.

FIGS. 8A-D. FIGS. 8A-B illustrate TM elastic modulus vs. donor age. AFM nanoindentation was used to determine the mean elastic modulus for (A) normal (inset: data plotted on smaller y scale) and (B) glaucomatous HTM samples. FIGS. 8C-D illustrate that force curve measurements taken every 50 microns on (C) normal (inset: data plotted on smaller y scale) and (D) glaucomatous TM. Young's modulus values of TM every 50 microns along the tissue show little variation in normal TM but a wide variation in the sample from the donor with glaucoma.

FIG. 9 illustrates an AFM tip probing a TM cell on a topographically patterned surface (1200 nm Pitch). The AFM tip (black) is brought in contact with the TM cell to determine modulus. The substrate patterned with ridges and grooves of 1200 nm pitch (pitch=ridge and groove width) can be discerned in the background. TM cells elongate and align with these features having biomimetic size scale. Bar=20 μm.

FIG. 10 illustrates effect of latrunculin on trabecular meshwork cells. TM cells from a 66 y.o. human donor were labeled with fluorescent wheat germ agglutinin. The modulus of the cells was measured with the AFM. Latrunculin (0.2 μM final) was added and the modulus was measured after 30 minutes. Left. Fluorescent and transmitted light image of TM cells before the addition of latrunculin. The modulus of the cells was 2.0 kPa. Right. Fluorescent and transmitted light image of TM cells after 30 minutes of latrunculin. Note the rounding of the cells. The modulus of the cells at this time was 0.07 kPa. Scale=50 μm.

FIGS. 11A-B illustrate that increased mRNA (A) and protein expression (B) of myocilin in TM cells occurs in response to biophysical cues. Real-time PCR was used to determine the relative levels of mRNA in TM cells (from three different human donors) for myocilin (A; left side). In all cases, expression of myocilin was increased substantially when cells were grown on 400 nm pitch surfaces. Addition of dexamethasone to cells grown on flat surfaces increased myocilin mRNA, but the addition of dexamethasone to cells grown on topographically patterned surfaces having features of biomimetic scale significantly increased myocilin levels. The increases in mRNA caused increases in myocilin protein expression (B; right side). Western blots of the TM 631 cells showed that increases in protein expression were similar to mRNA expression. Not all genes were influenced by biophysical cues as seen with αB-crystallin expression. P=planar control, 4=400 nm pitch control, PD=planar with dexamethasone, and 4D=400 nm pitch with dexamethasone.

FIG. 12 illustrates an increase in transglutaminase-2 gene expression in TM cells grown on stiffer hydrogels. The mRNA level of transglutaminase is increased when TM cells were grown on substrates with a Young's modulus of 100 kPa compared to cells grown on hydrogels of 3 kPa. The value of transglutaminase gene expression was normalized to 1.0 for the cells grown on the 3 kPa hydrogels.

FIG. 13 illustrates the modification of gene splicing in TM cells is directed by biophysical cues. Versican is a proteoglycan with chondroitin sulfate binding sites. The V0 isoform has the most binding sites with the V3 having zero. On 400 nm pitch topography, the V0 and V1 isoforms have increased expression of mRNA about 2.5 fold each compared to TM cells grown on planar surfaces. Note that the splicing of the V2 and V3 isoforms does not appear to be influenced by topography. Addition of dexamethasone in cultures appeared to negate the effect of the topographically patterned surfaces having biomimetic length scale. Control Planar=Cells grown on planar surfaces. Control 400P=Cells grown on 400 nm pitch surfaces. Dex Planar=Cells on planar surfaces cultured with dexamethasone. Dex 400P=Cells grown on 400 nm pitch surfaces cultured with dexamethasone.

FIGS. 14A-D illustrate immunofluorescence of fibronectin expression in TM cells on substrates with different compliance. TM cells cultured on glass (A and C; 30 GPa) show more expression of fibronectin (A and B; green) in a 24 hour time period than cells cultured on polyacrylamide gels (B and D; 30 kPa). Cells were also stained with phalloidin to reveal actin (red) and DAPI for cellular nuclei (blue) (C and D). The composite image with the three stains is shown below the fibronectin stain for cell boundary delineation.

FIG. 15 illustrates matrix metalloproteinase (MMP) and myocilin gene expression in TM cells is modulated by the compliance of the underlying substrate. TM cells were grown on either tissue culture plastic (3 GPa) or 30 kPa polyacrylamide gels in either the presence or absence of dexamethasone. Real-time PCR was used to quantify the expression of MMP-2, MMP-3 and myocilin. MMP-2 expression was similar on both substrates, but the addition of dexamethasone decreased expression by about 3-fold in both cases. Dexamethasone had no effect on the expression of MMP-3, but there was a 5-fold increase in MMP-3 expression on the more compliant polyacrylamide surfaces. Dexamethasone increased myocilin mRNA on flat surfaces, but the more compliant gel elicited a 5 fold increase in expression without added steroid and about a 20-fold increase with dexamethasone addition compared to the control cells grown on stiff plastic.

FIG. 16 illustrates phagocytosis of pHrodo BioParticles by TM Cells. The pHrodo BioParticles were incubated with TM cells for either 0 minutes or 4 hours. (A) Large concentrations of fluorescent particles (green) are seen in many cells at the four hour time point. (B) time course of phagocytosis measured by a fluorescent plate reader in TM cells at the two hour and four hour time point showing an increase in accumulated pHrodo Bioparticles over time.

FIG. 17 illustrates patterned substrate with pores support growth of TM cells. Scanning electron micrographs of our fabricated substrates patterned with pores with diameters of 30 nm (left) and part of a TM cell attached to the substrate after 24 hours.

FIG. 18 illustrates schematic of components of an AFM. The laser light bounces off the cantilever and is measured by the photodiode. Nanoindention of the sample is done by the tip of the cantilever. The cantilever deflection in volts is converted to deflection in nanometers. The conditions used for measurement had a cantilever frequency of 8-9 kHz, an applied cantilever voltage of 500 mV, a probe oscillation of 0.3-0.5 V and a slow scan rate of 0.3 Hz.

FIG. 19 illustrates a mathematical model of a flexible membrane with one micron holes as it becomes stiffer. Using the relationship for Poiseuille flow through the holes, the flow resistance of an aqueous solution is marked altered as the membrane becomes stiffer. Three curves are plotted with flows from 2 to 3 microliter per minute. This simplified example indicates facility is impacted as the TM becomes stiffer.

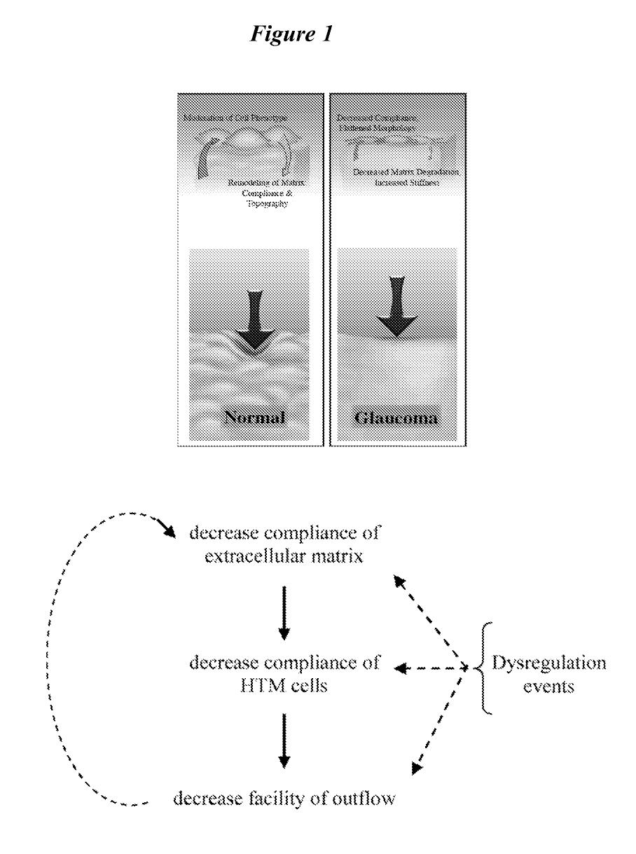

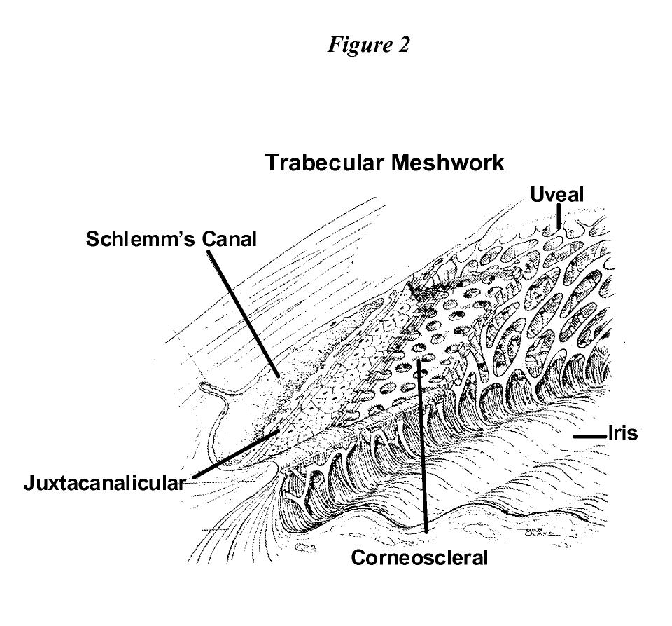

### DETAILED DESCRIPTION

### 1. Introduction

The present invention is based, in part, on the discovery that during progression of glaucoma; there are dysregulatory events that lead to stiffening (i.e., decreased compliance) of the extracellular matrix (ECM) and alterations in the HTM cell-substratum dynamic. Through cell-substratum interactions, the increased stiffness of the ECM increases the rigidity of overlying HTM cells and this change modifies the gene and protein expression profile of the cells and subsequently the ECM composition. Accordingly, the present invention provides methods for identifying agents that directly alter the compliance of the ECM in the trabecular matrix and/or the intrinsic compliance of cells within the HTM. The invention further provides methods for modulating compliance of the ECM and/or cells within the TM, for decreasing intraocular pressure and promoting outflow of fluid from within the aqueous humor, and for reducing the severity of glaucoma in a patient in need thereof by contacting the ECM and cells with the TM with one or more agents identified by the present screening methods.

### Properties of the Meshwork and Meshwork Cells

Alterations in the composition of the basement membrane and its organization modulate the surface topography and local compliance that are fundamental biophysical characteristics of the microenvironment of the HTM cell.

For example, as stated above, glaucoma is associated with an increased amount of transglutaminase. Increasing the relative amount of transglutaminase will increase the levels of cross-linked ECM proteins, which decreases local compliance (increased stiffness). The compliance of the glaucomatous meshwork may be lowered further by the apparent decrease in matrix γ-carboxyglutamic acid protein (MGP), a calcification inhibitor. Consistent with this finding, an increase in alkaline phosphatase, a marker for calcification, had been reported in association with glaucoma^{22,23}. With the changes in ECM composition, the ultrastructure of the glaucomatous meshwork is also changed²⁴. Glaucomatous eyes have elevated amounts of plaque-like material deposited in the JCT and an abundance of long spacing collagen²⁵. A correlation of the severity of optic nerve damage in glaucoma with changes in the trabecular meshwork has been established²⁶ indicating progression of visual loss is linked to alterations in the TM. In the aggregate, a number of ECM constituents of the HTM have been shown to be modulated in glaucomatous human eyes.

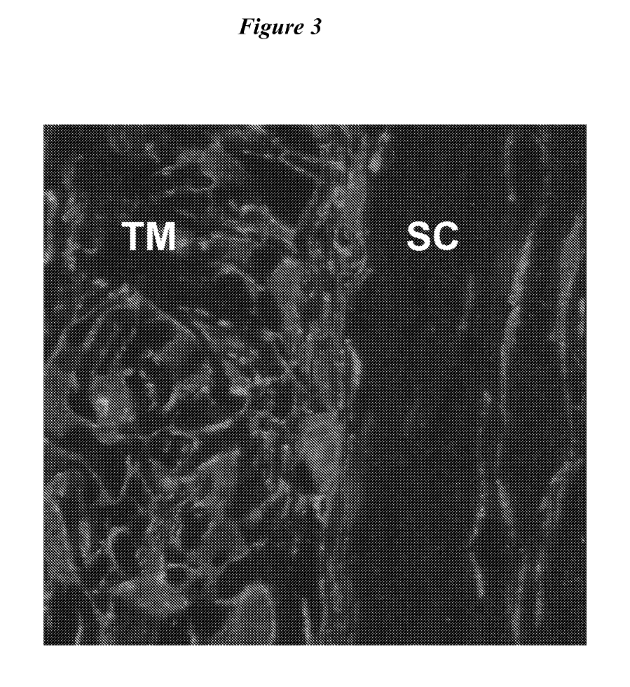

Matricellular proteins are expressed by HTM cells and cause alterations in the adhesion of cells to their underlining substratum. These proteins can also influence cell to cell interactions. For example, HTM cells produce SPARC (secreted protein, acidic, cysteine-rich (osteonectin)) at high levels²⁷ (FIG. 3). Intraocular pressure (IOP) in SPARC-null mice is reduced compared to normal control animals. Myocilin also acts as a matricellular protein modulating cellular adhesion^{28,29,30}. Myocilin, a secreted protein and a member of the olfactomedin family, is inducible in the HTM in response to glucocorticoids³¹. Myocilin is present in human aqueous humor and is synthesized by HTM cells^{32,33}. In the eye of a transgenic mouse that overexpresses myocilin at levels five fold higher than present in human aqueous humor³⁴ some changes in gene expression were measured but there was no change in IOP. With higher myocilin levels, decreases in the expression of genes Wasl were observed, coding for N-WASP, and Ceacam 1, a mediator of homophilic intercellular binding; and increases in expression of Spon2, a member of matricellular thrombospondin type 1 repeat proteins. Taken together, these data suggest the possibility that high expression of myocilin in human aqueous humor may diminish cell adhesion and may increase outflow facility. Further the data herein are consistent with the conclusion that myocilin is dramatically altered by the biophysical properties of the substratum.

The trabecular meshwork undergoes constant remodeling¹⁶, and the matrix metalloproteinases (MMPs) are an integral part of this process Inhibitors of MMPs have been demonstrated to reduce outflow; therefore, the remodeling process is important maintaining normal aqueous humor dynamics^{35,36}. MMP-2 and -14 are highly expressed in HTM with slightly lower expression of MMP-3. In vitro, changes in the MMP levels in HTM cells have been studied after mechanical stretch^{37,38}. Mechanical stretch simulates the type of distention that occurs to the meshwork in vivo and involves alterations in the cytoskeleton of HTM cells. The present invention provides methods for identifying agents that modulate MMP expression and activity, and consequently, the compliance of the ECM within the HTM, as well as HTM cell behavior.

Phagocytosis also plays a central role in remodeling of the ECM of the HTM. In perfused organ culture, HTM cells challenged with blood, latex microspheres or zymosan granules ingested all three types of particles and were retained in the meshwork. Animal studies indicate HTM cells after phagocytic challenge eventually migrated from the TM and were lost³⁹. Data suggest that reduced amounts of fibronectin and laminin are present in the meshwork after a phagocytic challenge, and this results in decreased levels of protein for cell attachment to the ECM40. The amount of phagocytosis in vivo may also involve a pro-inflammatory signal since less phagocytosis was observed in eyes in organ culture than in fellow eyes in vivo⁴¹. Dexamethasone, a compound used to study glucocorticoid induced glaucoma, causes an inhibition of phagocytosis⁴². Addition of γ-interferon also inhibits phagocytosis⁴³, suggesting that actin remodeling was involved in this inhibition. The relationship between actin and phagocytosis may be explained by the regulation in recruitment of actin involved with the phagosomal cup formation⁴⁴. Maintenance of the ECM is a dynamic and complex process, and failure to appropriately balance remodeling activities will lead to disease. Accordingly, alterations in the biophysical properties of the substratum of HTM cells alter the HTM cell-ECM dynamic through modulation of ECM production, density of ECM crosslinking as well as HTM mediated remodeling of the ECM by MMPs and phagocytic activity.

Modeling Glaucoma with HTM Cells

Donor glaucomatous tissue is highly variable with lower HTM cell numbers being able to be harvested than from normal HTM tissue. HTM cells from these donors also have lower proliferation rates than normal HTM and are more senescent^{45,46,47,23,48}. With the lack of a good animal model for the predominant forms of glaucoma in humans, HTM cells in culture are commonly used to test hypotheses concerning the initiation and progression of the disease. There are several systems that try to model changes in the TM using HTM cells. Four existing in vitro model systems have been used to model differing aspects of changes that have been reported in glaucomatous HTM, none of which consider the compliance of HTM cells or the ECM within the HTM.

1. Glucocorticoid Induced Changes:

One of the first systems studied extensively involved the use of glucocorticoids particularly dexamethasone. Glucocorticoids are known to increase IOP in a number of individuals and were shown to specifically induce the expression of myocilin in HTM cells^{49,50}. Unfortunately, the mRNA for myocilin is downregulated once HTM cells are placed in a standard in vitro tissue culture environment. However, myocilin levels will increase in vitro when HTM cells are cultured with dexamethasone or when they are subjected to mechanical stretch. It is significant that, even under these conditions, the levels of myocilin mRNA that are induced are less than observed from intact HTM tissue⁵¹. Microarrays of HTM cells treated with or without dexamethasone have been reported, and numerous genes related to both intra- and extra-cellular proteins have significant changes in expression levels after treatment with this glucocorticoid^{52,53}. Interestingly, dexamethasone also increased fibronectin levels in cultures of HTM cells from donor tissue, a result that is consistent with the increased fibronectin deposition in the glaucomatous outflow pathway⁵⁴. Glucocorticoids have also been shown to alter actin within HTM cells by inducing the formation of cross-linked actin networks. These networks have also been observed in the TM from glaucomatous eyes^{55,56,57}. While the dexamethasone system has revealed important insights into changes in the HTM cells, one of the difficulties with this system is that several of the mRNA and protein alterations only occur after many days. Thus, it is difficult to relate these alterations with specific pathways in the HTM cells.

2. Mechanical Stretch:

Another in vitro model for POAG is the mechanical stretch of HTM cells. This is commonly accomplished by either increasing the IOP in organ cultured HTM or by inducing cellular stretch on polymeric sheets upon which HTM cells have been seeded. Distention and stretching of the outflow tissue occurs in response to increased IOP. In addition to the alterations in cytoskeleton and ECM, at least 11 genes were found to be upregulated with stretch associated with increased IOP among them interleukin-6 and αB-crystallin⁵⁸. Interleukin-6 expression, induced by mechanical stretch of HTM, results in a 30% increase in outflow facility⁴⁶. αB-crystallin, a stress protein, has been shown to be increased in glaucomatous HTM. This stretch model for POAG points to alterations in the ECM and indicates facility can be altered by subtle changes in the composition of the matrix. The HTM actin cytoskeleton and signal transduction pathways, such as the MAP kinase pathway, were altered with a mechanical stretch of 10%⁵⁹. Stretch also results in alternative splicing of a number of genes of the ECM⁶⁰. These splice variants will alter the composition of the ECM, potentially altering outflow. Although versican as a whole is decreased with stretch, the V1 isoform is increased almost four fold. These data suggest not only gene expression but also gene splicing may be altered with glaucoma. The induction of shear stress in this model provides biophysical cues that are distinct from the biophysical cues presented to the HTM cells by providing substrates with biomimetic compliance.

3. TGF-β Induced Changes:

The TGF family includes the bone morphogenetic proteins (BMP). In the HTM, there appears to be a balance between TGF-β and BMPs^{61,62}. Although gremlin, an inhibitor of the actions of BMPs, is present in the eye, exogenous addition of this inhibitor in a perfused anterior segment caused increased IOP. Both gremlin mRNA and protein are elevated in glaucomatous HTM cells suggesting a correlation between BMP inhibition and disease⁶³. In the aqueous humor of individuals with POAG, there are elevated levels of TGF-β2⁶⁴. Thus the balance between the TGF-β and the BMPs is altered in glaucoma. Both TGF-β1 and TGF-β2 can change expression of genes, which influence the cytoskeleton or the ECM⁶⁵. TGF-β will induce αB-crystallin consistent with the increase in this protein in the JCT with glaucoma⁶⁶. Not only will TGF-β2 decrease MMP activity but it will also induce tissue transglutaminase causing irreversible cross-linking of ECM proteins^{12,67}. In perfused anterior segments, TGF-β2 elevated IOP and increased fibronectin content⁶². Thrombospondin-1, a potent activator of TGF-β, is expressed in the aqueous humor outflow pathways and can be induced by either TGF-β1 or dexamethasone⁶⁸.

4. Reactive Oxygen Species Induced Changes:

Reactive oxygen species can be generated as a result of a NF-κB/IL-1 positive feedback loop⁶⁹. IL-1 will increase the levels of MMP-3 both in HTM cells and in the HTM after laser trabeculoplasty^{62,70,71,72}. A sustained positive feedback loop could negate the positive influence of MMP-3 and could cause oxidative ECM modification. Such a sustained stress response has been shown to cause increases in αB-crystallin, IL-6 and vascular endothelial leukocyte adhesion molecule (ELAM-1)^{73,74}.

While these four models have been widely used, they possess significant limitations. No model provides for the appropriate biophysical cues that mimic those encountered in vivo. It is possible that one of the reasons that in vitro results frequently fail to translate to the in vivo conditions is the failure to appropriately model the biophysical properties of the microenvironment of the HTM cells.

### ECM, Outflow Function and Therapeutics

The ECM is important to the outflow pathway. This matrix is constantly being remodeled by HTM cells and disruption of this remodeling process or alterations in the components of the ECM of the basement membrane can cause decreases in outflow facility. Data indicate that medications that just reduce the perfusion of aqueous humor through the HTM are detrimental to facility in the long run^{75,76,77,78}. Therefore, the best therapeutic drugs to lower IOP should work on not reducing inflow or increasing flow through the unconventional outflow pathway, but rather these drugs should cause increased outflow through the trabecular meshwork. Unfortunately, all the drugs currently marketed do not target the HTM. Data suggest that although they decrease pressure, long term effects of these agents will result in reduced facility. Some of the new treatment modalities targeted to the HTM indicate that disruption of the cytoskeleton will increase facility and will reduce IOP^{79,80,81,82,83}. Recent work on the cytoskeleton and ECM shows that there is an interdependence between the two^{84,85}.

### Biophysical Cues and HTM Cell Behaviors

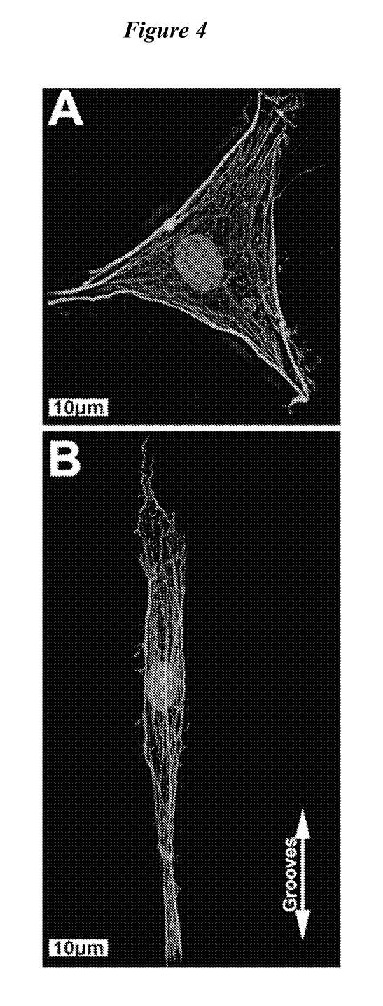

Most work to date has completely disregarded the biophysical cues delivered to HTM cells via the ECM, but rather has focused on exposing cells to soluble biochemical cues. Studies mimicking one of the biophysical cues present in basement membrane, namely surface topography, drove dramatic increases in the expression of myocilin in HTM cells⁸⁶. This single element of the biophysical environment was sufficient to increase myocilin levels expressed in HTM cells to levels that approximate those observed in vivo. Besides the change in protein expression, the cytoskeleton of the HTM cells is also altered by this singular biophysical cue. Intracellular actin architecture is dramatically altered by the provision of substratum topographic cues (FIG. 4). Another fundamental component of the biophysical environment is the compliance of the substratum. As the compliance decreases, the rigidity of the substance increases. Data on the compliance of the HTM indicates normal values of around 3 kiloPascals (kPa). This is similar to many other basement membranes but is substantially different than the GigaPascals (GPa) compliance characteristic of glass or tissue culture plastic. HTM cells grown on soft but uncharacterized substrates indicates cytoskeletal dynamics, protein expression, ECM composition and signal transduction are all altered by varying compliance⁸⁷. In order to create more relevant in vitro systems that model aspects of glaucoma utilizing HTM cells, the present invention provides TM cells with a biophysical environment that mimics the one they sense in vivo. This allows an understanding of how modifications of the ECM delineated in work on the glaucomatous meshwork causes changes in the HTM cells that influence the progression of this disease. Biochemical studies without the incorporation of biophysical parameters are relying on only two cues (biochemical and cell-to-cell) and disregard the importance of the biophysical signals that the HTM cells receive. The presence of biophysical cues can dramatically alter the response of cells to biochemical signals present in the extracellular environment^{88,86}.

### Biophysical Cues—Compliance

The vast majority of studies on the effect of environmental stimuli have been carried out using stiff culture substrates such as silicon, polystyrene, titanium and polyurethane. However, compliance is emerging as an important property of substrates that can impact cell behaviors. The compliance of a material relates the extent of deformation (strain) of the material to an imposed stress (force/unit area). Mechanical compliance of substrates such as membrane supports has been shown to dramatically modify the responses of cells⁸⁹. Tactile sensing of substrate stiffness modulates adhesion processes and cytoskeletal dynamics⁹⁰. Substrate stiffness influences cell behaviors including adhesion, proliferation, migration and differentiation^{91,84,92,93}.

Cells possess an intrinsic elastic modulus that can vary according to cell type as well as the compliance of the underlying substrate to which they are attached. In general, the cellular moduli range from 0.4 to 5 kPa⁹⁴. Data for HTM cells show that on substrates having compliance values in the range of 3 kPa, the cells have a modulus value of around 1.1 kPa. In marked contrast, a HTM cell modulus of 5.5 was measured for cells attached to glass substrates (50 GPa). Our observations demonstrating variations in cell compliance dependent on the compliance of the underlying substrate are supported by work with other cells. Fibroblasts will generally match the substrate stiffness up to about 5 kPa without formation of stress fibers⁹⁵. Above this substrate modulus, fibroblasts increase their rigidity as a result of cytoskeletal changes, including stress fiber formation, until they reach a saturating rigidity. It is presumed that the cells reach a limit on mechanisms to reinforce their cytoskeleton. Phosphorylation of myosin light chain has been implicated in this process^{96,97}. These results motivated subsequent studies on the mechanism involved in transducing the mechanical signal into a cellular signal. MAPK activity was shown to be significantly higher on cells grown on stiffer substrates⁹⁸. In addition, migration exhibited a biphasic dependence on substrate stiffness with cultured smooth muscle cells. Cells cultured on substrates lower than 50 kPa had migration speeds of around 0.42 μm/min, while on surfaces more rigid, the cells has speeds of 0.72 μm/min.

In certain conditions such as diabetes and heart disease, there is a dysregulation of the ECM. Progression of disease leads to increased expression of matrix proteins or increased alterations of ECM proteins resulting in increased substrate rigidity^{99,100,101}. Certain drugs such as glucocorticoids can influence the cytoskeleton and change the modulus of the cells directly¹⁰². In HTM, glucocorticoids also change the ECM components which could easily result in decreased compliance as a result of cross-linking of proteins²¹.

To date, a systematic investigation of the effect of substrate compliance on HTM cells has not been conducted. The present invention provides information for understanding the dynamic relationship between biophysical properties of the ECM and their consequences on HTM cells.

### Biophysical Cues—Topography

The surfaces of normal basement membranes have rich 3-dimensional nanoscale topography. Quantitative studies of the topographic features of corneal basement membranes of the human, Rhesus Macaque and dog, of the vascular endothelium of Rhesus Macaque aorta and carotid, of the aortic valve of the pig, of the Rhesus Macaque urinary bladder and of matrigel, a commercially available basement membrane-like complex have been completed^{107,108,109,110}. Basement membranes possess a “felt-like” architecture consisting of pores, intertwining fibers and bumps. The surface topography of normal basement membranes is largely conserved in nature both across species and anatomic locations. (Table 1).

TABLE 1
Basement Membrane Topographic Features
are Similar Across Species and Anatomic Locations.

| | Tissue Basement | Mean Fiber | Mean Pore |
|---|---|---|---|
| Species | Membrane | Diameter (nm) | Diameter (nm) |
| Rhesus | Cornea | 77 ± 44 | 72 ± 40 |
| Human | Cornea | 46 ± 16 | 92 ± 34 |
| Rhesus | Aorta | 30 ± 11 | 62 ± 37 |
| Rhesus | Bladder | 52 ± 28 | 82 ± 49 |
| Synthetic | Matrigel | 69 ± 35 | 105 ± 70 |

Note
that the sizes of the fiber diameter and the pore size diameter are very similar from one basement membrane to another.

For each basement membrane analyzed, individual feature dimensions reside in the sub-μm (100-1000 nm,) to nanoscale (1-100 nm) range¹¹⁰. This is also supported by reports of basement membranes of the kidney^{111,112,113} and qualitative reports of the basement membrane of human skin¹¹². These studies provide context in which to interpret the effects of topographic cues on cell behavior as well as gene and protein expression. The rich nanoscale and submicron architecture ensures that a single cell interfaces with thousands of topographic features of the underlying basement membrane.

Numerous reports document surface topography of substrates modulating cell behaviors. However, the majority of the surfaces that have been investigated to date used techniques to create well-defined patterned topographies with surface features ≧1 μm. Recently with advances in materials sciences, the generation of large numbers of substrates with controlled fabrication of nanoscale and sub-micron surface features have become possible that enable statistically robust in vitro experiments. Nanoscale features, comparable to the dimension of a single collagen fiber, activate macrophage cell adhesion and subsequent spreading, and alter the amount of F-actin in cells^{114,115}. The orientation of extracellular matrix components has been shown to affect the spatial orientation and morphology of cells. Nanoscale topographic features have been documented to directly affect cell differentiation^{88,116,117}. Investigators have also used porous filters to evaluate the effect of substratum pores on cell behavior. Porous filters (with pore sizes ranging from 100 nm to 3 μm and porosity ranging from ˜3-16%) have been shown to modulate epithelial and fibroblast cell behavior in vitro. This range of pore sizes and periodicities is comparable to that described for native basement membranes. Pores with diameters of 100 nm to 800 nm and porosities of 2.5-12% provided the best surface for cell migration and differentiation^{118,119,120,121}.

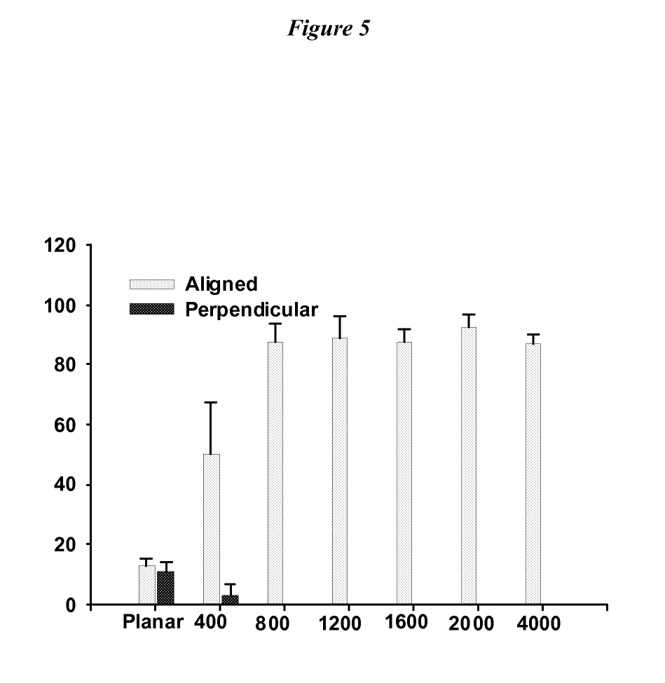

Previous work has focused on the modulation of corneal epithelial cell behavior by nano, submicron and micropatterned substratum topographic features^{109,122}. The approach used is unique in that it is motivated by detailed quantitative description of native basement membranes. Using the topography of the native basement membrane as a guide, anisotropically ordered silicon surfaces that contain a range of feature sizes ranging from 400 nm to 4000 nm in pitch (pitch=groove width+ridge width) with intervening planar regions were designed and fabricated. The smallest feature sizes on these chips were 70 nm ridges on a 400 nm pitch. This allowed the replication on a single chip features that mimicked the biologically relevant nano and submicron length scale of native basement membranes. These chips also contain micron-scale structures that allowed investigation of transitions in cell behaviors between micro and nanoscale features and also provide a link to the bulk of the literature. Although the substrates are not identical to native basement membrane with regards to the orientation of the topographic features, the anisotropically ordered substrates closely mimic the feature dimensions of native structures and allow for unambiguous analysis of contact guidance and other experimental endpoints. Using these chips, it was found that HTM cellular orientation is most profoundly modulated by topographic features with biologic length scale dimensions⁸⁶. Alignment of primary HTM cells is parallel to the long axis on all feature sizes (FIG. 5). In other cell lines differences in cell behavior have been noted at the nano and submicron scale. In the presence of low concentrations of nerve growth factor, PC12 cell neuritogenesis was approximately 3-fold greater when cells were grown on nanoscale topographical features compared to cells grown on planar or micron sized features. This finding shows that topography can act synergistically with sub-optimal growth factor signaling to promote a cellular response⁸⁸. For example, biomimetic cues on HTM impact cellular gene expression, such as myocilin.

A number of synthetic surfaces have been shown to be toxic for cells and data suggested that, in some cases, the toxicity was due to the physical interaction of these substrates with cells rather than due to the release of toxic leachables¹²⁸. In one study, non-textured surfaces were used and their data suggested “surface toxicity” may be related to the inability of the surfaces evaluated to promote cell stretching thus promoting entry into apoptosis¹²⁴. The literature documents that changes in cell volume (which alters with changes in cell shape) affects the activities of certain genes. Some of these genes code for cytoskeletal elements and heat-shock proteins. Several of the genes activated are of a general regulatory type and influence the activity of a wide array of other genes¹²⁹. Taken together, these data document dramatic effects of nano-submicron scale topography on cell behaviors (characteristic of native ECM) and suggest several mechanisms that may be involved in transducing biophysical events at the cell membrane.

Because of the lack of good animal models for glaucoma, investigations into the biological mechanism of the disease process have been done with HTM cells in culture. The preponderance of the studies has utilized cells grown on flat tissue culture plastic. Most of these studies have used biochemical cues to elicit changes in cell behaviors. However, biophysical cues (topography and compliance) also have profound effects on cell behaviors. The biophysical cues received by HTM cells have not been well studied yet these cues undoubtedly influence biochemically mediated behaviors. A better understanding of the interactions of the biophysical and biochemical cues would give more meaningful analysis of HTM cell behaviors to shape strategies for ameliorating the effect of glaucoma.

### 2. Culturing Trabecular Meshwork In Vitro

a. Culturing TM Cells

Trabecular meshwork tissue can be obtained from donor corneal buttons of normal or glaucomatous subjects. Methods for culturing cells within TM tissue are known in the art (See, e.g., Rhee, et al., Exp Eye Res. 2003; 77:749-756). For example, TM cells can be grown in DMEM/F-12 media with 20% fetal bovine serum. Preferably, the TM cells are used in the screening assays before the seventh passage.

The TM cells can be cultured on any surface that supports their growth. The culture surface selected will influence the baseline compliance value or elastic modulus. TM cells cultured on stiff culture substrates, e.g., silicon, polystyrene, titanium and polyurethane, will have decreased compliance. TM cells cultured on soft materials will have increased compliance.

Studies exploring the impact of substratum compliance on cell behavior often take advantage of the versatile properties of a class of materials known as hydrogels. Hydrogels find use as scaffolds for tissues. In vitro studies have demonstrated the utility of hydrogels used as cell culture substrates. Hydrogels have water retention characteristics comparable to that of native human tissues. A variety of hydrogel systems with varying compliance have been used to evaluate the effects of the physical environment on cell behavior^{89,103,89} and protein binding¹⁰⁴. Hydrogels that find use as culture surfaces for TM cells include without limitation those based upon poly(ethylene glycol)diacrylate (PEGDA), poly (2-hydroxyethylmethacrylate) (PHEMA), collagen I, collagen IV, elastin, hyaluronic acid (HA), polyacrylamide and hybrids of these components. Gelation, resulting in a covalently cross-linked network, can be accomplished by free radical polymerization. The chemical structure of hydrogels allows manipulation of their physical properties, including a range of compliances. Hydrogel flexibility can be controlled by a number of strategies. For example, differing values for compliance can be accomplished by changing the cross-linker density of polymer chains, changing the molecular weight of the pre-polymer chains, and changing the chemistry of the gels. The range of compliance values obtainable in hydrogels is wide, from near liquid through the rubbery region to glassy polymers, a range that will encompass the biomimetic values of 2 kPa-250 MPa¹⁰⁵. Preferred hydrogels have a Young's modulus in the range of about 3 kPa to about 100 kPa.

In some embodiments, the TM cells are cultured on a textured surface, e.g., with fabricated patterns. Substrates comprising a range of substratum features including grooves, ridges, pores, steps, wells, nodes, and adsorbed protein fibers find use. Numerous fabrication strategies can be employed to create textured substrates with a focus on photolithography^{109,122,130,88,131,132}.

Nanotextured surfaces with controlled and reproducible feature sizes can be fabricated. The textured area for each experiment can be at least a few mm² to perform assays such as proliferation, adhesion, migration, morphology, and differentiation assays. Larger substrates may be needed (up to 50 to 100 cm²) to perform Western blot or gene chip assays. A wide variety of master stamps containing the grooves, ridges, pores, wells and nodes have been made. These stamps range from the nanometer to micron size range with precise shapes, sizes and distributions. The composite stamping replication procedures developed by Whitesides¹³³ have been used to generate thousands of substrates in our laboratory^{131,132}. Polyurethane and hydrogel substrates have been successfully fabricated with these stamping protocols. Polyurethane substrates with anisotropic grooves and ridges have been extensively studied in the laboratory. These surfaces are optically transparent and can be used to culture cells and allow for real-time imaging. Substrates that integrate topographic patterns into hydrogels of differing compliance to provide a full menu of surfaces with biologically relevant biophysical properties can be created.

b. ECM

For determining compliance values of ECM, it is not necessary to use TM tissue. ECM from other bodily tissues find use. Also, ECM from different mammalian species can be used, e.g., humans, non-human primates, porcine.

For culturing extracellular matrix from the trabecular meshwork, TM tissue from donor corneal buttons are dissected. In the fluid commonly used for transplant materials, the TM tissue is usable for a couple of weeks. It is preferable to determine the compliance of the TM tissue as soon as possible after dissection, because the TM tissue starts to degenerate upon dissection. Compliance measurements of TM tissue include TM cells and ECM together. Measurements generally do not involve fixation so the ECM or the cells can be kept in PBS or media. The cell component of TM tissue is minor, so the compliance value of TM tissue is predominantly contributed by the ECM.

### 3. Contacting Trabecular Meshwork with an Agent

In the screening assays of the invention, the TM tissue or cultured TM cells is contacted with an agent. The TM tissue or cultured TM cells is exposed to a sufficient amount of test agent under conditions to allow the test agent to effect a change in compliance.

Functionally, test agents that modulate the compliance of a TM cell may disrupt the cytoskeleton of the TM cell, for example, by disrupting intermediate filaments, microtubules and/or actin polymerization. Further test agents that find use may modulate gelation of the cytosol (independent of altering cytoskeletal dynamics) or the stiffness of the lipid bilayer of the cell.

Functionally, test agents that modulate the compliance of the ECM may influence the level of cross-linking of ECM components, the expression levels of matricellular proteins, the expression levels or functionality of enzymes that degrade, erode or alter the ECM, or the ratio of ECM components (e.g., the ratio of hyaluronic acid (HA) to chondroitin SO₄). Agents that increase the compliance of the ECM in TM tissue increase the expression of matricellular proteins (e.g., myocilin, SPARC), increase the expression levels and/or functionality of enzymes that degrade, erode or alter the ECM (e.g., elastase, MMPs and/or ADAMTs), decrease the expression levels and/or functionality of enzymes that cross-link the ECM (e.g., transglutaminases) or increase the ratio of hyaluronic acid (HA) to chondroitin SO₄ (e.g., decrease versican expression).

Structurally, the test agents can be, e.g., small organic compounds (e.g., molecular weight less than 10,000 daltons, for example, less than 8000, 6000, 4000, 2000 daltons), polypeptides (e.g., peptides, antibodies, antigen binding molecules, aptamers), nucleic acids (e.g., short inhibitory mRNA (siRNA), micro RNA (miRNA), antisense nucleic acids, ribozymes, aptamers, plasmids), carbohydrates and lipids. Typically, the amount of inhibitory agent contacted with the TM tissue or cultured TM cells is from about 0.05 nM to about 50 μM, for example, about 1 nM to about 1 μM, about 0.1 μM to about 50 μM, or about 0.05 nM, 0.1 nM, 0.5 nM, 1.0 nM, 1.5 nM, 25 nM, 100 nM, 1.0 μM, 10 μM, or 50 μM.

Exposure times of the test agents to the TM cells and/or tissue can on the order of one or several hours (e.g., 1, 2, 3, 4, 5, 6, 7, 8 or more hours); however, agents can be taken up by HTM cells and work for extended periods (e.g., overnight, 12, 24, 36 or 48 hours, as appropriate).

Organic Compounds

In some embodiments, the one or more agents are small organic compounds. Essentially any chemical compound can be screened for its effects on the compliance of TM tissue or cultured TM cells. Most preferred are generally compounds that can be dissolved in aqueous or organic (especially DMSO-based) solutions and compound which fall within Lipinski's “Rule of 5” criteria. The assays are designed to screen large chemical libraries by automating the assay steps and providing compounds from any convenient source to assays, which are typically run in parallel (e.g., in microtiter formats on multiwell plates in robotic assays). It will be appreciated that there are many suppliers of chemical compounds, including Sigma-Aldrich (St. Louis, Mo.); Fluka Chemika-Biochemica Analytika (Buchs Switzerland), as well as numerous providers of small organic molecule libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), Tripos, Inc. (St. Louis, Mo.), Reaction Biology Corp. (Malvern, Pa.), Biomol Intl. (Plymouth Meeting, Pa.), TimTec (Newark, Del.), and AnalytiCon (Potsdam, Germany), among others.

Organic compounds known to disrupt the cytoskeleton of a cell can be tested for their effect on the compliance of a cultured TM cell. For example, one or more organic compounds that disrupt intermediate filaments, microtubules, or actin polymerization can be screened for its effects on the compliance of a cultured TM cell. Exemplary organic compounds that disrupt intermediate filaments include without limitation acrylamide, rho kinase inhibitors and blebbistatin. Exemplary organic compounds that disrupt microtubules include without limitation colchicine, colecemid, vinca alkaloids (e.g., vinblastine, vincristine, vinorelbine, vindesine), podophyllotoxin, capecotobine, nocodazole, tryprostatin A, rhizoxin, vinflunine, epothilones, ixabepilone, methyl benzimidazol-2-yl-carbamate, estramustine sodium phosphate, taxanes (e.g., paclitaxel, docetaxil, colchitaxel), and indibulin. Exemplary organic compounds that disrupt actin polymerization include without limitation cytochalasin B, cytochalasin D, latrunculin A, latrunculin B, migrastatin, cucurbitane-type tritepenes B&E, olivetoric acid, chivosazole A, chivosazole F and desmethoxymajusculamide C.

The organic compound can also be a substrate analog of an MMP or ADAMTS enzyme (as described herein), wherein the substrate analog increases the catalytic activity of the enzyme. In other embodiments, the organic compound is a substrate analog of a transglutaminase that decreases the catalytic activity of the enzyme.

Known organic compounds determined to modulate the compliance of TM tissue and/or cultured TM cells find therapeutic use, discussed herein, and also use as a core chemical structure upon which a chemical library can be based, i.e., to develop further derivatives having similar or improved functionality in modulating compliance.

Polypeptides and Antibodies

In some embodiments, the one or more agents are polypeptides (including but not limited to peptides having 8-30 amino acids, antibodies, etc.). Polypeptide libraries useful for screening are commercially available from numerous sources, for example, from Cambridge Peptides, Cambridge, United Kingdom; JPT Peptide Technologies, Berlin, Germany; Bio•Synthesis, Lewisville, Tex.; and Prestwick Chemical, Washington, D.C. Methods for producing peptide libraries are also well known in the art. See, for example, Synthetic Peptides: A User's Guide, Grant, ed., 2002, Oxford University Press; Benoiton, Chemistry of Peptide Synthesis, 2005, CRC Press; Jones, Amino Acid and Peptide Synthesis, 2002, Oxford University Press. Peptide synthesizers are commercially available, for example, from TechniKrom, Inc., Evanston, Ill.; Applied Biosystems, Foster City, Calif.; and Advanced Automated Peptide Protein Technologies (AAPPT), Louisville, Ky.

In some embodiments, the one or more agents are antibodies, including polyclonal or monoclonal antibodies, Fab fragments, single chain antibodies (scFv), complementary regions from combinatorial libraries, etc. A combinatorial antibody library useful for screening purposes is available from MorphoSys, Martinsried/Planegg, Germany. Methods for producing antibody libraries are known in the art. Non-antibody antigen binding molecules also find use as agents. Exemplary non-antibody antigen binding molecules include without limitation antibody mimics that use non-immunoglobulin protein scaffolds, including adnectins, avimers, anticalins, single chain polypeptide binding molecules, and antibody-like binding peptidomimetics.

Polypeptides known to disrupt the cytoskeleton of a cell can be tested for their effect on the compliance of a cultured TM cell. For example, one or more polypeptides that disrupt intermediate filaments, microtubules, or actin polymerization can be screened for its effects on the compliance of a cultured TM cell. Exemplary polypeptides that disrupt intermediate filaments include without limitation calpain-1 (NM_{—}005186.2→NP_{—}005177.2), calpain-2 (NM_{—}001146068.1→NP_{—}001139540.1 (calpain-2 catalytic subunit isoform 2); NM_{—}001748.4→NP_{—}001739.2 (calpain-2 catalytic subunit isoform 1; NM_{—}032330.1→NP_{—}115706.1 (calpain small subunit 2)), caldesmon (NM_{—}033138.3→NP_{—}149129.2 (isoform 1); NM_{—}033139.3→NP_{—}149130.1; NM_{—}004342.6→NP_{—}004333.1 (isoform 2); NM_{—}033157.3→NP_{—}149347.2 (isoform 3); NM_{—}033139.3→NP_{—}149130.1 (isoform 4); NM_{—}033140.3→NP_{—}149131.1 (isoform 5)). Exemplary polypeptides that disrupt microtubules include without limitation dolastatin 15. Exemplary agents that disrupt actin polymerization include without limitation E47 transcription factor for semaphorin 3F, semaphorin 3F (NM_{—}004186.3→NP_{—}004177.3), actin depolymerizing factor (NM_{—}001011546.1→NP_{—}001011546.1 (destrin isoform b); NM_{—}006870.3→NP_{—}006861.1 (destrin isoform a)), gelsolin (NM_{—}001747.2→NP_{—}001738.2 (macrophage-capping protein)) clostridium perfringens iota, and clostridium botulinum C2.

Known polypeptides determined to modulate the compliance of TM tissue and/or cultured TM cells find therapeutic use, discussed herein, and also use as a starting polypeptides upon which fragments, mutated polypeptides, or a peptide library can be based, i.e., to develop further polypeptides having similar or improved functionality in modulating compliance.

Inhibitory Oligonucleotides

In some embodiments, the one or more agents are inhibitory oligonucleotides, including antisense oligonucleotides, ribozymes, short inhibitory RNA (siRNA), micro RNA (miRNA). Libraries of randomized oligonucleotides are commercially available from, for example, Integrated DNA Technologies (IDT), Coralville, Iowa; Ambion, Austin, Tex. and Qiagen, Valencia, Calif. Inhibitory nucleic acids that find use inhibit the expression.

Inhibitory nucleic acids that inhibit the expression and/or activity of transglutaminase, versican, or other enzymes involved in the cross-linking of ECM find use. Inhibitory nucleic acids that inhibit the expression of effector molecules that inhibit MMPs, ADAMs, ADAM-TSs and other enzymes that degrade or modify the extracellular matrix find use. Such effector molecules include without limitation inhibitors of Tissue Inhibitors of Matrix Metalloproteinases (TIMPs) that inhibit enzyme activity. Exemplary TIMPs include without limitation TIMP1 (NM_{—}003254.2→NP_{—}003245.1), TIMP2 (NM_{—}003255.4→NP_{—}003246.1), TIMP3 (NM_{—}000362.4→NP_{—}000353.1) and TIMP4 (NM_{—}003256.2→NP_{—}003247.1).

Antisense Oligonucleotides

An “antisense” oligonucleotide corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vitro and in vivo conditions. The antisense molecule must have sufficient complementarity to the target gene so that the antisense RNA can hybridize to the target gene (or mRNA) and inhibit target gene expression regardless of whether the action is at the level of splicing, transcription, or translation. In some embodiments, the complementary antisense sequence is about 15-30 nucleotides in length, for example, 15, 16, 17, 18, 19, 20, 21, 22, 23, 24, 25, 26, 27, 28, 29 or 30 nucleotides, or longer or shorter, as desired. The antisense components of the present invention may be hybridizable to any of several portions of the target cDNA, including the coding sequence, 3′ or 5′ untranslated regions, or other intronic sequences, or to target mRNA.

Antisense oligonucleotides can include sequences hybridizable to any of several portions of the target DNA, including the coding sequence, 3′ or 5′ untranslated regions, or other intronic sequences, or to target mRNA.

Small Inhibitory RNA Oligonucleotides

siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. siRNA can be effected by introduction or expression of relatively short homologous dsRNAs. For screening purposes, the double stranded oligonucleotides used to effect inhibition of expression, at either the transcriptional or translational level, can be of any convenient length. siRNA molecules are typically from about 15 to about 30 nucleic acids in length, for example, about 19-25 nucleic acids in length, for example, about 15, 16, 17, 18, 19, 20, 21, 22, 23, 24, 25, 26, 27, 28, 29, 30 nucleic acids in length. Optionally the dsRNA oligonucleotides can include 3′ overhang ends. Exemplary 2-nucleotide 3′ overhangs can be composed of ribonucleotide residues of any type and can be composed of 2′-deoxythymidine resides, which lowers the cost of RNA synthesis and can enhance nuclease resistance of siRNAs in the cell culture medium and within transfected cells (see, Elbashi et al., 2001, Nature 411:494-8).

Longer dsRNAs of 50, 75, 100 or even 500 base pairs or more can also be utilized in certain embodiments of the invention. Exemplary concentrations of dsRNAs for effecting inhibition are about 0.05 nM, 0.1 nM, 0.5 nM, 1.0 nM, 1.5 nM, 25 nM or 100 nM, although other concentrations can be utilized depending upon the nature of the cells treated, the gene target and other factors readily discernable to the skilled artisan.

Exemplary dsRNAs can be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see, e.g., Elbashir, et al., 2001, Genes Dev. 15:188-200). Alternatively the dsRNAs can be transcribed from a mammalian expression vector. A single RNA target, placed in both possible orientations downstream of an appropriate promoter for use in mammalian cells, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the siRNA oligonucleotides can be any contiguous sequence of nucleotides contained within the expressed gene message of the target. Programs and algorithms, known in the art, may be used to select appropriate target sequences. See, the Ambion website at ambion.com. In addition, optimal sequences can be selected utilizing programs designed to predict the secondary structure of a specified single stranded nucleic acid sequence and allowing selection of those sequences likely to occur in exposed single stranded regions of a folded mRNA. Methods and compositions for designing appropriate siRNA oligonucleotides may be found, for example, in U.S. Pat. No. 6,251,588, the contents of which are incorporated herein by reference.

Ribozymes

Ribozymes are enzymatic RNA molecules capable of catalyzing specific cleavage of RNA. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Inhibitory oligonucleotides can be delivered to a cell by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells.

### 4. Determining Compliance of a Trabecular Meshwork

Compliance (inverse of stiffness) is a physical parameter that has a profound effect on a number of cell behaviors. The compliance of human trabecular meshworks (HTM) from both normal and glaucomatous donors can be determined. The local compliance can be measured using any method known in the art. For example, any technique that applies a stress and measure the resultant strain finds use. Exemplary methods for determining compliance include without limitation indenting (e.g., AFM, nanoindenters, instrons), pulling (e.g., instrons), compressing (e.g., instrons, AFM, nanoindenters), shearing (e.g., rheometers, AFM), bending (e.g., instrons), buckling (e.g., instrons), wave propagation (e.g., acoustic or ultrasonic; can use many different wavelengths), and drilling.

Methods to characterize the compliance of soft materials commonly utilize bulk samples in tension or compression testing. However, these methods oftentimes do not accurately reflect the local compliance. In preferred embodiments, AFM scanning probe techniques¹⁰⁶ are employed to characterize the local modulus of the surface of soft tissues, hydrogels, TM tissue and cultured TM cells. Determination of local compliance for cell substratum investigations using highly sensitive AFM techniques provides a more accurate characterization of the micro-, submicro- and nano-scale environment that the cells experience.

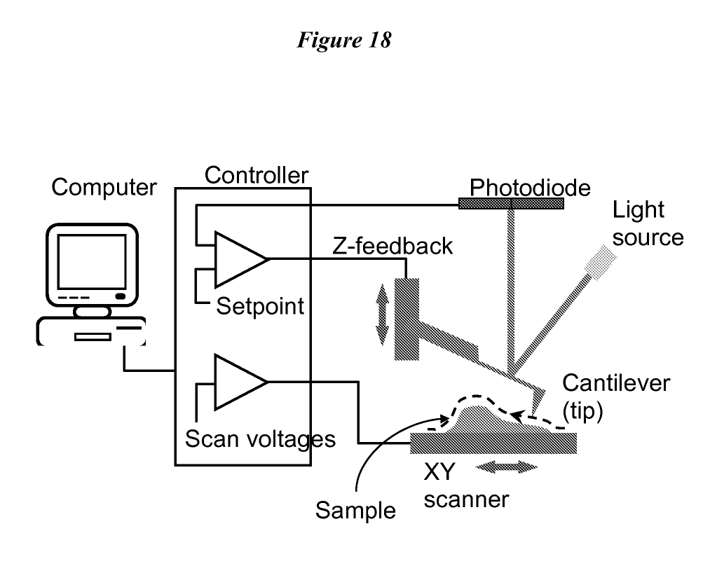

Accordingly, in some embodiments, the intrinsic or local compliance of the TM can be determined using an atomic force microscope (AFM). FIG. 18 shows a schematic of components of an AFM. Commercially available AFM find use in the present screening assays, e.g., from Asylum Research (Santa Barbara, Calif.), Veeco (veeco.com), Novascan (Ames, Iowa), Park Systems, (Santa Clara, Calif.). The parameters for measuring the compliance of a cultured TM cell include a cantilever frequency of 8-9 kHz, an applied cantilever voltage of 500 mV, RMS amplitude (probe oscillation) of 0.3-0.5 V, and a slow scan rate (0.3 Hz). Compliance can be measured with commercially available AFM probes with a 1 μm radius spherical tip (the approximate dimension of a focal adhesion complex). The force curves can be taken at a rate of 2 μm/sec. The resulting curves can be analyzed using the Hertz model to obtain the compliance. Different indentor spring constants, or probe geometries can be used for probing cells versus. tissues, as appropriate. For example, a very weak spring to probe can be used to probe cellular mechanics, whereas a comparatively stiffer spring can be used to probe cartilage.

The force curves generated by the AFM can be used to calculate the Young's modulus of the HTMs using the Hertz equation. With these results, a mathematical model of a flexible membrane containing open spaces (pores) of 1 micron was developed to determine how outflow resistance is influenced. The following model considers the biomechanical and biofluidic responses of the primary aqueous outflow resistance at the juxtacanalicular meshwork (JCT). This model demonstrates the utility of the compliance measurements described herein in understanding changes occurring in vivo after exposure to agents that alter cytoskeletal and/or ECM dynamics with concomitant alterations in cellular rigidity. In this model, the JCT is simplified as an elastic membrane structure with parallel cylindrical micropores imbedded. With intraocular pressure (IOP), the aqueous flow passes through the JCT layer and expands the porous structure outwards. The flow resistance (R) of JCT can be mathematically calculated using the Poiseuille's law:

$$R = \frac{Δ P}{Q} = \frac{8 μ t}{NA π {r}^{4}} = \frac{8 πμ t}{{NAA}_{c}^{2}} \qquad ( 1 )$$

where ΔP and Q are the intraocular pressure (IOP) and the aqueous humor flow rate, respectively, while μ represents the fluid viscosity. Other parameters are all structurally related parameters which include: A and t the overall area (10.8 mm2) and thickness of JCT (50 μm), N the porous density (350/mm2), r the pore radius (0.5 μm) and Ac the area of an individual micropore (0.785 μm) (Ethier, Johnson and Ruberti. Ann. Rev. Biomed. Eng 6:249-273, 2004).

Furthermore, Ac can be modeled as dependence on the Young's modulus of JCT (E) as well as the aqueous humor flow rate (Q):

A_{c}=f(Q,E) (2)

Therefore, combining both Eq. 1 and 2, the membrane resistance can be established in the following form:

$$R = \frac{8 π μ t}{NA} \frac{1}{{f ( Q , E )}^{2}} \qquad ( 3 )$$

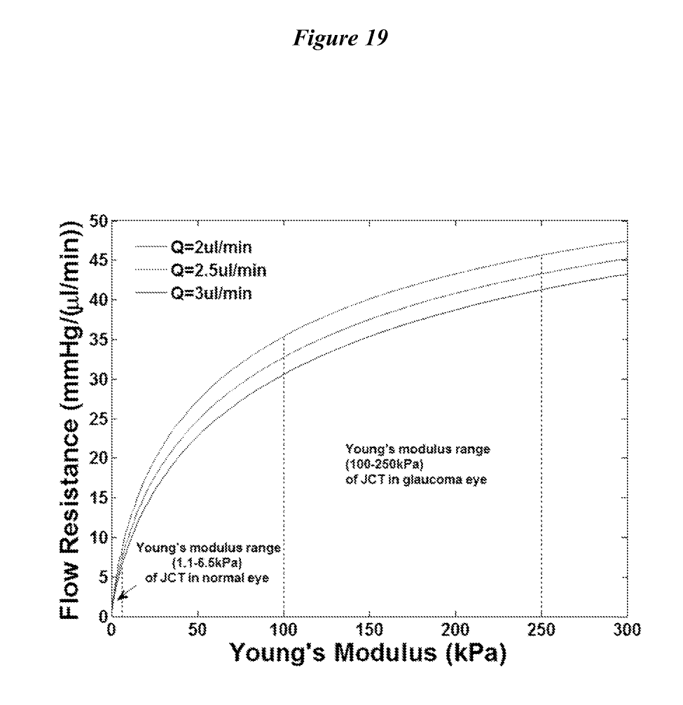

According to the above equation, the aqueous outflow resistance is plotted in FIG. 19. Considerable difference in flow resistance between the normal and glaucomatous eyes can be seen as a result of abnormal stiffness of the JCT. In addition, increasing the outflow rate lowers the flow resistance of the JCT. Elevated IOP established by increased flow can further enlarge the pore size and reduce the flow resistance of the JCT. This model uses established engineering formulae found in the text Theory of Plates and Shells 2nd Edition by Timoshenko and Woinowsky-Krieger. This model applies to correlating the drop in modulus in HTM cells with outflow through the JCT. If the drop in modulus of the HTM at the JCT mirrors that of the cell, changes in the JCT in vivo can be predicted.

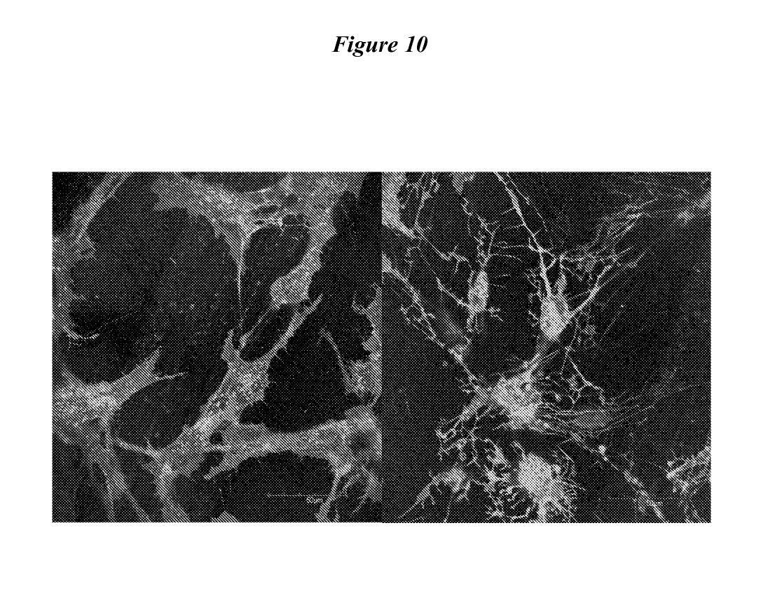

The model is consistent with the data presented herein that altering the biomechanical properties of the JCT alters outflow dynamics. First, the stiffness of the JCT in glaucomatous eyes is markedly increased (Table 2. Second, the stiffness of normal HTM cells is increased when in contact with stiffer substrates (Table 3). Third, the intrinsic stiffness of HTM cells is markedly decreased on exposure to latrunculin (FIG. 10). In the aggregate, the data and the model above are consistent with the conclusion that increasing the JCT stiffness in glaucoma is associated with increased stiffness of the HTM cells and together this increases resistance to outflow which can be addressed directly by exposure to agents, e.g., that disrupt the cytoskeleton or remodel the extracellular matrix.

### 5. Comparison to a Reference Point

The compliance value or Young's modulus determined for the test TM tissue or test cultured TM cell is compared to a reference point.

In some embodiments, the compliance value or Young's modulus determined for the test TM tissue or test cultured TM cell is compared to an untreated control TM tissue or control cultured TM cell, i.e., that has not been exposed to the test agent. Preferably, the test TM tissue or test cultured TM cell and the untreated control TM tissue or control cultured TM cell are from the same originating TM tissue. The agent is deemed to decrease compliance if the Young's modulus of the test TM tissue or test cultured TM cell is at least about 10% greater, e.g., at least about 20%, 25%, 30%, 50%, 75%, 100% greater, after exposure to the agent than the Young's modulus of the control TM tissue or test cultured TM cell. The agent is deemed to increase compliance if the Young's modulus of the test TM tissue or test cultured TM cell is at least about 10% smaller, e.g., at least about 20%, 25%, 30%, 50%, 75%, 100% smaller, after exposure to the agent than the Young's modulus of the control TM tissue or test cultured TM cell.

In some embodiments, the compliance value or Young's modulus is determined for the same test TM tissue or test cultured TM cell before and after exposure to the test agent. The agent is deemed to decrease compliance if the Young's modulus of the test TM tissue or test cultured TM cell is at least about 10% greater, e.g., at least about 20%, 25%, 30%, 50%, 75%, 100% greater, after exposure to the agent than the Young's modulus of the same TM tissue or test cultured TM cell prior to exposure to the agent. The agent is deemed to increase compliance if the Young's modulus of the test TM tissue or test cultured TM cell is at least about 10% smaller, e.g., at least about 20%, 25%, 30%, 50%, 75%, 100% smaller, after exposure to the agent than the Young's modulus of the same TM tissue or test cultured TM cell prior to exposure to the agent.

In some embodiments, the compliance value or Young's modulus determined for the test TM tissue or test cultured TM cell is compared to a threshold compliance value or Young's modulus, e.g., a predetermined compliance value based on the measurements of a population of TM tissues and/or cultured TM cells. Threshold values can be determined based on the compliance measurements of a population of TM tissues and/or cultured TM cells from normal and glaucomatous individuals. Accordingly, in such assays, it is generally known whether the TM tissue is from a normal or non-glaucomatous versus a glaucomatous individual. The agent is deemed to decrease compliance if the Young's modulus of the test TM tissue or test cultured TM cell is at least about 10% greater, e.g., at least about 20%, 25%, 30%, 50%, 75%, 100% greater, after exposure to the agent than the predetermined threshold Young's modulus. The agent is deemed to increase compliance if the Young's modulus of the test TM tissue or test cultured TM cell is at least about 10% smaller, e.g., at least about 20%, 25%, 30%, 50%, 75%, 100% smaller, after exposure to the agent than the predetermined threshold Young's modulus.

### 6. Methods of Treatment and Prevention

The invention provides methods of reducing intraocular pressure in a subject in need thereof, comprising administering to the subject an effective amount of an agent that increases the compliance of a TM cell and/or an agent that increases the compliance of an ECM in the TM, wherein the outflow of aqueous humor through the TM is increased, thereby reducing intraocular pressure in the subject. Further provided are methods of reducing the severity (e.g., reversing or delaying the progression) of glaucoma in a subject in need thereof, comprising administering to the subject an effective amount of an agent that increases the compliance of the TM cell and/or an agent that increases the compliance of the ECM in the TM, wherein the outflow of aqueous humor through the TM is increased, thereby reducing the severity of glaucoma in the subject. In some embodiments, it may be of therapeutic benefit to an individual to administer an agent that decreases the compliance of the TM. The agent that is administered can be identified according to the methods described herein.

a. Conditions Subject to Treatment and Prevention

The eye does not collapse because the pressure within the eye (the “intraocular pressure,” or “IOP”) is greater than that of the surrounding atmosphere. Normally, the IOP is between 10-20 mm Hg greater than the pressure of the atmosphere, although there is some modest daily fluctuation. IOP is created by the aqueous humor, a clear fluid that enters the anterior chamber of the eye via the ciliary body epithelium (inflow), flows through the anterior segment bathing the lens, iris, and cornea, and then leaves the eye via specialized tissues known as the trabecular meshwork (TM) and Schlemm's canal to flow into the venous system. Intraocular pressure is maintained by a balance between fluid secretion and fluid outflow. According to the NEI, most glaucomas result from a defect in the outflow and a subsequent buildup of pressure.

IOP is usually measured by determining the resistance of the eye to an external force. A variety of instruments are used to measure IOP clinically, including the Goldmann tonometer, which uses a prism to flatten the cornea, the Tono-Pen® XL applanation tonometer (Medtronic Xomed Ophthalmics, Inc., Jacksonville, Fla.), a hand-held device containing a plunger, and the Schiotz tonometer, which measures the indentation of the cornea produced by a weight.

Pharmacological agents that increase the compliance of TM tissue facilitate outflow of aqueous humor fluid through the TM, and therefore find use in the treatment (e.g., reversal or delay of progression) and prevention of eye disorders characterized by high intraocular pressures. Accordingly, patients who will benefit from administration of an agent that increases the compliance of TM tissue may present with intraocular pressure of greater than 20 mm Hg above ambient atmospheric pressure. The patient may have glaucoma and/or have been diagnosed by a clinician as having glaucoma.

In some embodiments, the patient has a normal IOP and is undergoing a therapeutic regime of another medication currently used to ameliorate symptoms of glaucoma. Independent of presenting IOP, the patient may be diagnosed as having glaucoma and under a therapeutic regime of an agent to treat glaucoma, e.g., a prostaglandin analog (e.g., latanoprost, bimatoprost, or travoprost); a topical beta-adrenergic receptor antagonist (e.g., timolol, levobunolol, and betaxolol); an alpha2-adrenergic agonist (e.g., brimonidine); a sympathomimetic (e.g., epinephrine, dipivefrin); a miotic agent (e.g., pilocarpine, ecothiopate); a carbonic anhydrase inhibitor (e.g., dorzolamide, brinzolamide, acetazolamide); or physostigmine. Other agents are described herein.

Individuals having increased IOP but who do not have clinically defined glaucoma can also benefit from administration of an agent that increases compliance of the TM.

b. Agents for Administration

Pharmacological agents for administration include those that increase the compliance of the trabecular meshwork, either at the level of the ECM or individual cells within the TM. The agents may be known compounds used for other indications, or new compounds identified by the screening methods described herein.

In some embodiments, an agent that disrupts the cytoskeleton of a TM cell is administered according to the methods described herein. Known agents that disrupt the cytoskeleton of a cell can be administered. For example, one or more agents that disrupt intermediate filaments, microtubules, or actin polymerization can be administered. Exemplary agents that disrupt intermediate filaments include without limitation acrylamide, calpain-1 (NM_{—}005186.2→NP_{—}005177.2), calpain-2 (NM_{—}001146068.1→NP_{—}001139540.1 (calpain-2 catalytic subunit isoform 2); NM_{—}001748.4→NP_{—}001739.2 (calpain-2 catalytic subunit isoform 1; NM_{—}032330.1→NP_{—}115706.1 (calpain small subunit 2)), rho kinase inhibitors, blebbistatin, caldesmon (NM_{—}033138.3→NP_{—}149129.2 (isoform 1); NM_{—}033139.3→NP_{—}149130.1; NM_{—}004342.6→NP_{—}004333.1 (isoform 2); NM_{—}033157.3→NP_{—}149347.2 (isoform 3); NM_{—}033139.3→NP_{—}149130.1 (isoform 4); NM_{—}033140.3→NP_{—}149131.1 (isoform 5)), and inhibitory RNA (RNAi) that inhibits expression of proteins involved in formation and maintenance of intermediate filaments. Exemplary agents that disrupt microtubules include without limitation colchicine, colecemid, vinca alkaloids (e.g., vinblastine, vincristine, vinorelbine, vindesine), podophyllotoxin, capecotobine, dolastatin 15, nocodazole, tryprostatin A, rhizoxin, vinflunine, epothilones, ixabepilone, methyl benzimidazol-2-yl-carbamate, estramustine sodium phosphate, taxanes (e.g., paclitaxel, docetaxil, colchitaxel), and indibulin. Exemplary agents that disrupt actin polymerization include without limitation cytochalasin B, cytochalasin D, latrunculin A, latrunculin B, migrastatin, E47 transcription factor for semaphorin 3F, semaphorin 3F (NM_{—}004186.3→NP_{—}004177.3), actin depolymerizing factor (NM_{—}001011546.1→NP_{—}001011546.1 (destrin isoform b); NM_{—}006870.3→NP_{—}006861.1 (destrin isoform a)), cucurbitane-type tritepenes B&E, gelsolin (NM_{—}001747.2→NP_{—}001738.2 (macrophage-capping protein)), olivetoric acid, chivosazole A, chivosazole F, clostridium perfringens iota, clostridium botulinum C2, and desmethoxymajusculamide C.

In some embodiments, the agent administered increases the compliance of the ECM within the TM tissue. In some embodiments, the agent administered decreases the compliance of the ECM within the TM tissue.

In some embodiments, the administered agent increases expression or activity of matricellular proteins, for example, increases the expression of myocilin (NM_{—}000261.1→NP_{—}000252.1) and/or secreted protein, acidic, cysteine-rich (osteonectin) (SPARC) (NM_{—}003118.2→NP_{—}003109.1). In some embodiments, the administered agent decreases expression or activity of matricellular proteins, for example, decreases the expression of myocilin (NM_{—}000261.1→NP_{—}000252.1) and/or secreted protein, acidic, cysteine-rich (osteonectin or SPARC) (NM_{—}003118.2→NP_{—}003109.1).

In some embodiments, the administered agent modulates (e.g., increases or decreases) expression or activity of enzymes that degrade, erode or alter the ECM, including but not limited to matrix metalloproteinases (MMPs), disintegrin and metalloproteinases (ADAMs) and disintegrin and metalloproteinase with thrombospondin motifs (ADAM-TSs). Agents that increase compliance increase the expression or activity of enzymes that degrade, erode or alter the ECM. Exemplary MMPs include without limitation MMP-1 (e.g., NM_{—}001145938.1→NP_{—}001139410.1 (interstitial collagenase isoform 2); NM_{—}002421.3→NP_{—}002412.1 (interstitial collagenase isoform 1), MMP-2 (NM_{—}001127891.1→NP_{—}001121363.1 (72 kDa type IV collagenase isoform b); NM_{—}004530.4→NP_{—}004521.1 (72 kDa type IV collagenase isoform a), MMP-3 (NM_{—}002422.3→NP_{—}002413.1 (stromelysin-1)), MMP-9 (NM_{—}004994.2→NP_{—}004985.2), MMP-11 (NM_{—}005940.3→NP_{—}005931.2 (stromelysin-3)), MMP-12 (NM_{—}002426.4→NP_{—}002417.2), MMP-14 (NM_{—}004995.2→NP_{—}004986.1 (membrane-type MMP or MT1-MMP)), MMP-15 (NM_{—}002428.2→NP_{—}002419.1), MMP-16 (NM_{—}005941.4→NP_{—}005932.2 (matrix metalloproteinase-16 isoform 1); NM_{—}022564.3→NP_{—}072086.2 (matrix metalloproteinase-16 isoform 2)), MMP-17 (NM_{—}016155.4→NP_{—}057239.4) and MMP-19 (NM_{—}002429.4→NP_{—}002420.1 (matrix metalloproteinase-19 isoform rasi-1)).

Exemplary ADAMs include without limitation ADAM metallopeptidase domain 2 (ADAM2; NM_{—}001464.3→NP_{—}001455.3); ADAM metallopeptidase domain 7 (ADAM7; NM_{—}003817.2→NP_{—}003808.2); ADAM metallopeptidase domain 8 (ADAM8; NM_{—}001109.4→NP_{—}001100.3 (isoform 1); NM_{—}001164489.1→NP_{—}001157961.1 (isoform 2); NM_{—}001164490.1→NP_{—}001157962.1 (isoform 3)); ADAM metallopeptidase domain 9 (meltrin gamma or ADAM9; NM_{—}003816.2→NP_{—}003807.1); ADAM metallopeptidase domain 10 (ADAM10; NM_{—}001110.2→NP_{—}001101.1); ADAM metallopeptidase domain 11 (ADAM11; NM_{—}002390.4→NP_{—}002381.2); ADAM metallopeptidase domain 12 (ADAM12; NM_{—}003474.4→NP_{—}003465.3 (isoform 1); NM_{—}021641.3→NP_{—}067673.2 (isoform 2)); ADAM metallopeptidase domain 15 (ADAM15; NM_{—}003815.3→NP_{—}003806.3 (isoform 1); NM_{—}207191.1→NP_{—}997074.1 (isoform 2); NM_{—}207194.1→NP_{—}997077.1 (isoform 3); NM_{—}207195.1→NP_{—}997078.1 (isoform 4); NM_{—}207196.1→NP_{—}997079.1 (isoform 5); NM_{—}207197.1→NP_{—}997080.1 (isoform 6)); ADAM metallopeptidase domain 17 (ADAM17; NM_{—}003183.4→NP_{—}003174.3); ADAM metallopeptidase domain 18 (ADAM18; NM_{—}014237.1→NP_{—}055052.1); ADAM metallopeptidase domain 19 (meltrin beta or ADAM19; NM_{—}033274.2→NP_{—}150377.1); ADAM metallopeptidase domain 20 (ADAM20; NM_{—}003814.4→NP_{—}003805.3); ADAM metallopeptidase domain 21 (ADAM21; NM_{—}003813.2→NP_{—}003804.1); ADAM metallopeptidase domain 22 (ADAM22; NM_{—}004194.2→NP_{—}004185.1 (isoform 4); NM_{—}016351.3→NP_{—}057435.2 (isoform 3); NM_{—}021721.2→NP_{—}068367.1 (isoform 5); NM_{—}021722.3→NP_{—}068368.2 (isoform 2); NM_{—}021723.2→NP_{—}068369.1 (isoform 1)); ADAM metallopeptidase domain 23 (ADAM23; NM_{—}003812.2→NP_{—}003803.1); ADAM metallopeptidase domain 28 (ADAM28; NM_{—}014265.4→NP_{—}055080.2 (isoform 1); NM_{—}021777.3→NP_{—}068547.2 (isoform 3)); ADAM metallopeptidase domain 29 (ADAM29; NM_{—}001130703.1→NP_{—}001124175.1 (variant 2); NM_{—}001130704.1→NP_{—}001124176.1 (variant 3); NM_{—}001130705.1→NP_{—}001124177.1 (variant 4); NM_{—}014269.4→NP_{—}055084.3 (variant 1)); ADAM metallopeptidase domain 30 (ADAM30; NM_{—}021794.2→NP_{—}068566.2); ADAM metallopeptidase domain 33 (ADAM33; NM_{—}025220.2→NP_{—}079496.1 (isoform alpha); NM_{—}153202.1→NP_{—}694882.1 (isoform beta)).

Exemplary ADAM-TSs include without limitation ADAM-TS1 (NM_{—}006988.3→NP_{—}008919.3), ADAM-TS4 (NM_{—}005099.4→NP_{—}005090.3), and ADAM-TS5 (NM_{—}007038.3→NP_{—}008969.2).

The administered agent can be a substrate analog for an MMP, ADAM or ADAM-TS enzyme, wherein the analog increases the catalytic activity of the enzyme. Agents that increase compliance increase the catalytic activity of an MMP, ADAM or ADAM-TS enzyme. Agents that decrease compliance decrease the catalytic activity of an MMP, ADAM or ADAM-TS enzyme. In some embodiments, the administered agent is the enzyme itself or an enzymatically active variant thereof.

In other embodiments, the administered agent can modulate effector molecules that inhibit elastase, MMPs, ADAMs, ADAM-TSs and other enzymes that degrade or modify the extracellular matrix. Such an agent is exemplified by but is not limited to inhibitors of Tissue Inhibitors of Matrix Metalloproteinases (TIMPs) that inhibit enzyme activity. Exemplary TIMPs include without limitation TIMP1 (NM_{—}003254.2→NP_{—}003245.1), TIMP2 (NM_{—}003255.4→NP_{—}003246.1), TIMP3 (NM_{—}000362.4→NP_{—}000353.1) and TIMP4 (NM_{—}003256.2→NP_{—}003247.1). In one embodiment, the administered agent is an inhibitory nucleic acid (e.g., an siRNA) that inhibits the expression of one or more TIMPs and thus augments the degradative action of the ECM by native enzymes modulated by TIMPs. In another embodiment, the administered agent enhances promoter activation of a MMP, ADAM or ADAM-TS gene, thereby increasing endogenous activity of the enzyme. See, e.g., Brew, et al., (2000). Biochim Biophys Acta 1477 (1-2): 267-83; Lee, et al., J Biol Chem. (2005) 280(16):15967-75; and Seals and Courtneidge (2003) Genes & Dev. 17: 7-30.

The expression and activity of MMPs can be modulated by agents including prostaglandins, COX-2 inhibitors and tetracyclines. Generally, prostaglandins and COX-2 inhibitors up-regulate MMP activity. However, selective COX-2 inhibitors can inhibit expression of MMP9 and tetracyclines decrease MMP activity. Prostaglandin F(2 alpha) (PGF2alpha) inhibits expression of TIMP1, resulting in increased expression of MMPs See, e.g., Ricke, et al., (2002) Biology of Reproduction 66(3):685-691; Ito, et al., (2004) Cancer Research 64:7439-7446 and Steenport, et al., J Immunol. (2009) 183(12):8119-27.

In some embodiments, the administered agent decreases cross-linking of the ECM. For example, the agent may decrease the cross-linking of one or more ECM components, including but not limited to fibronectin or a collagen, for example, collagen I, collagen III, collagen IV and collagen VI. In some embodiments, the agent decreases or inhibits the expression and/or activity of a transglutaminase. For example, a substrate analog of a transglutaminase that decreases the catalytic activity of the enzyme can be administered.

In some embodiments, the administered agent increases the ratio of glycosaminoglycans hyaluronic acid (HA) to chondroitin SO₄. For example, the agent may decrease the expression of a versican isoform, a chondroitin sulfate proteoglycan.

The patient may also benefit from combination therapies, co-administering a regime of two or more agents with distinct targets or mechanisms of action within the trabecular meshwork tissue. For example, the first agent can be a cytoskeleton disruptor (e.g., at the level of the intermediate filaments, the microtubules or actin polymers) and the second agent can effect remodeling of the ECM. In another example, two or more different agents that disrupt different aspects the cytoskeleton can be administered. In a further example, an agent that inhibits decrease aqueous humor production can be combined with an agent that promotes outflow through the TM. Also, an agent that promotes uveoscleral outflow of aqueous humor can be combined with an agent that promotes outflow through the TM.

Agents that modulate the compliance of TM tissue find use in combination therapies with one or more anti-glaucoma agents. Exemplary anti-glaucoma agents include without limitation brimonidine, brimonidine tartrate, brinzolamide ophthalmic suspension, levobunolol hydrochloride ophthalmic solution, betaxolol HCL, dorzolamide hydrochloride ophthalmic solution, dorzolamide hydrochloride-timolol maleate ophthalmic solution, timolol maleate ophthalmic solution, timolol maleate ophthalmic gel forming solution, dichlorphenamide, acetazolamide epinephrine, apraclonidine, carbachol, bimatoprost ophthalmic solution, methazolamide, carteolol hydrochloride ophthalmic solution, metipranolol ophthalmic solution, echothiophate iodide, pilocarpine, dipivefrin hydrochloride, unoprostone isopropyl ophthalmic solution, travoprost ophthalmic solution, latanoprost ophthalmic solution, and latanaprost and timolol ophthalmic solution. Other anti-glaucoma agents are described herein.

Additionally, agents that modulate compliance of the TM can be used in combination with therapeutic agents that increase outflow through effects on cytoskeletal disruption aimed at relaxing the TM (which is distinct from altering the intrinsic compliance of cells and/or extracellular matrix) and/or disrupting intercellular adhesions. Such compounds are exemplified by but not limited to non-corneotoxic macrolides, such as latrunculin-A, latrunculin-B, swinholide-A, and/or jasplakinolide. See, e.g., U.S. Pat. Nos. 6,586,425; 6,110,912; and 5,798,380.

c. Formulation

The pharmacological agents that increase compliance of TM tissue can be prepared and administered in a wide variety of formulations for administration to the eyes. The formulations can be introduced onto or into the eye by, for example, applying the formulation to the eyelids or to the conjunctival sac in aqueous or viscous solutions or suspensions, in ointments, in small pellets, as fine powders, on cotton pledgets, by drug-impregnated contact lenses, by injection, by mechanical pumps, or by membrane release systems. In preferred forms, compounds for topical use in the methods of the present invention can be administered as eye drops, ointments, or small pellets to be placed under the eyelids. Accordingly, the methods of the invention permit administration of pharmaceutical compositions comprising a pharmaceutically acceptable carrier or excipient and a selected pharmacological agent, or combination thereof.

The pharmacological agents can be administered systemically (e.g., usually orally, but also intravenously, buccally, subcutaneously and via other systemic routes, as appropriate) or locally (e.g., topically, onto the eye directly or onto tissues around the eye; directly in and/or around the TM tissue; or intraocularly).

Administration of pharmacologically active agents to the eyes is well known, and considerable information is set forth in standard works, such as Zimmerman et al. (eds.), TEXTBOOK OF OCULAR PHARMACOLOGY, Lippincott Williams & Wilkins (1997); Jannus et al., (eds.), CLINICAL OCULAR PHARMACOLOGY, Butterworth-Heinemann (4th Ed., 2001), and Mauger and Craig, HAVENER'S OCULAR PHARMACOLOGY, Mosby-Year Book (6th Ed., 1994), Grosvenor, PRIMARY CARE OPTOMETRY, Butterworth-Heinemann, (4th Ed., 2001), Duvall and Kerschner, OPHTHALMIC MEDICATIONS AND PHARMACOLOGY, SLACK Inc., Thorofare, N.J. (1998), and Fechner and Teichmann, OCULAR THERAPEUTICS: PHARMACOLOGY AND CLINICAL APPLICATION, SLACK Inc., Thorofare, N.J. (1997). These well known techniques can be readily applied to prepare and administer agents that increase the compliance of TM tissue to persons in need thereof.

For preparing pharmaceutical compositions, pharmaceutically acceptable carriers can be either solid or liquid. The carriers may also act, for example, as diluents, binders, or preservatives.

Liquid form preparations include solutions, suspensions, and emulsions, for example, water or water/propylene glycol solutions. Other typical forms for administration of the agents, or combinations thereof are liquid paraffin, polyvinyl alcohol, povidine, carbomers, hypromellose, hydroxyethylcellulose, hydroxypropylcellulose, and carboxymethylcellulose.

Formulations for intravitreous injection are also known in the art. Intravitreal injection is typically performed in the outpatient setting using topical anesthesia and a small-bore needle (e.g., 27 or 30 gauge) to deliver the medication into the vitreous cavity of the eye via the pars plana portion of the globe. Typically, the agents, or combinations thereof are administered as a sterile, preservative-free aqueous solution, which may optionally contain sodium chloride, monobasic sodium phosphate monohydrate, dibasic sodium phosphate heptahydrate, hydrochloric acid, and/or sodium hydroxide and other agents to adjust the viscosity and pH.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as vials or ampoules.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of one or more agents is employed in reducing intraocular pressure, e.g., for slowing or reversing the progression of glaucoma. The dosage of the specific compound for treatment depends on many factors that are well known to those skilled in the art. They include for example, the route of administration and the potency of the particular compound.

In some aspects of the invention, the agent, or combinations thereof, is dissolved or suspended in a suitable solvent, such as water, ethanol, or saline, and administered as an aerosol of fine particles by breaking a fluid into fine droplets and dispersing them into a flowing stream of gas. Typically, such aerosols develop approximately 15 to 30 microliters of aerosol per liter of gas in finely divided droplets with volume or mass median diameters in the range of 2 to 4 micrometers. Predominantly, water or saline solutions are used with low solute concentrations, typically ranging from 1.0 to 5.0 mg/mL.

As noted, drugs may be applied to the eyelids or instilled in the conjunctival sac in aqueous or viscous solutions or suspensions, in ointments, as fine powders, on cotton pledgets, by drug-impregnated contact lenses, by injection, by mechanical pumps, or by membrane release systems. In contrast to systemic administration, the ocular concentration after topical administration is high. Dilution of the drug by tears, overflow onto the cheek, and excretion through the nasolacrimal system limit tissue concentration. Placing the drug beneath a contact lens, applying a cotton pledget, or applying a collagen shield saturated with the drug to the eye prolongs the contact and aids penetration.

d. Dosing and Scheduling

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. Generally, an efficacious or effective amount of one or more agents is determined by first administering a low dose or small amount of the agent and then incrementally increasing the administered dose or dosages, and/or adding a second agent as needed, until a desired effect of inhibiting or preventing high intraocular pressures or symptoms of glaucoma is observed in the treated subject, with minimal or no toxic side effects. Applicable methods for determining an appropriate dose and dosing schedule for administration of a pharmaceutical composition of the present invention is described, for example, in Goodman and Gilman's The Pharmacological Basis of Therapeutics, 11 th Ed., Brunton, et al., Eds., McGraw-Hill (2006), and in Remington: The Science and Practice of Pharmacy, 21st Ed., University of the Sciences in Philadelphia (USIP), Lippincott Williams & Wilkins (2005), both of which are hereby incorporated herein by reference.

Dosage amount and interval can be adjusted individually to provide plasma or tissue levels of the agent sufficient to maintain a therapeutic effect. Single or multiple administrations of the compositions comprising an effective amount of one or more agents can be carried out with dose levels and pattern selected by the treating physician. The dose and administration schedule can be determined and adjusted based on the severity of the glaucoma or high intraocular pressure in the subject, which can be monitored throughout the course of treatment according to methods commonly practiced by clinicians or those described herein. In some embodiments, therapeutic levels will be achieved by administering single daily doses. In other embodiments, the dosing schedule can include multiple daily dose schedules. In still other embodiments, dosing every other day, semi-weekly, or weekly are included in the invention.

In embodiments where the agent is a polypeptide or an antibody, typical dosages can range from about 0.1 μg/kg body weight up to and including about 1 gm/kg body weight, preferably between about 1 μg/kg body weight to about 500 mg/kg body weight. More preferably, about 1, 2, 3, 4, 5, 10, 20, 30, 40, 50, 60, 70, 80, 90, or 100 mg/kg body weight.

In embodiments where the agent is a nucleic acid, typical dosages can range from about 0.1 mg/kg body weight up to and including about 100 mg/kg body weight, preferably between about 1 mg/kg body weight to about 50 mg/kg body weight. More preferably, about 1, 2, 3, 4, 5, 10, 15, 20, 30, 40 or 50 mg/kg body weight.

In embodiments were the agent is a small organic compound, typical dosages can range from about 0.1 μg/kg body weight up to and including about 1 gm/kg body weight, preferably between about 1 μg/kg body weight to about 500 mg/kg body weight. More preferably, about 0.1, 1, 2, 3, 4, 5, 10, 20, 30, 40, 50, 60, 70, 80, 90, or 100 mg/kg body weight.

The exact dose will depend on a variety of factors as discussed supra, including the particular inhibitor, severity of the disease, and route of administration. Determining the exact therapeutically effective dose can be determined by a clinician without undue experimentation and can include any dose included within the ranges disclosed above.

In cases of local administration or selective uptake, the effective local concentration of the agent may not be related to plasma concentration. One having skill in the art will be able to optimize therapeutically effective local dosages without undue experimentation.

Without further elaboration, it is believed that one skilled in the art can, using the preceding description, practice the present invention to its fullest extent.

### EXAMPLES

The following examples are offered to illustrate, but not to limit the claimed invention.

### Example 1

### Materials and Methods

Sample Preparation.

Normal human trabecular meshwork (HTM) cells were dissected from donated, unused corneal buttons using published protocols¹³⁹. Human donor eyes or corneal buttons determined unsuitable for transplantation were obtained from several eye banks. In general, the glaucomatous samples were obtained without detailed information on the type of glaucoma, the extent of disease or the medications used for treatment. Information with regards to the type of glaucoma was provided with only 3 of the 10 glaucoma samples, each with a diagnosis of primary open angle glaucoma. The ages of the donors ranged from 32 to 92 years. The tissues were stored in Optisol at 4° C. prior to dissection to remove the trabecular meshwork. Samples were prepared by dissection of the iris and uveal tissue with an ophthalmic knife (Alcon Surgical, Fort Worth, Tex.). The trabecular meshwork was sectioned with a razor blade to provide samples less than 1 cm in length and the sections were removed from the angle with forceps. The tissue was oriented so that the Schlemm's canal side of the juxtacanalicular tissue (JCT) was probed and was adhered by the trabecular region with cyanoacrylate glue in the center of a stainless steel AFM disk. AFM analysis was performed in 1× phosphate buffered saline (PBS). The average time from donation to analysis was 17.5±10.7 days for normal and 7.4±4.2 days for glaucomatous samples.

In order to characterize the region of tissue investigated with the AFM, multiple normal corneal buttons were processed for histopathology at various stages of preparation. Donor tissues were processed for routine histology as: 1. wedges of intact limbus; 2. wedges of limbus after sharp dissection of uveal tissue; 3. isolated trabecular meshwork (longitudinal and cross section); and 4. wedges of limbus after trabecular meshwork isolation. All samples were fixed for 24-48 hours and processed routinely, and sections of isolated HTM were oriented for either longitudinal or cross-sectional sampling. After embedding, samples were sectioned at 4 μm and stained with hematoxylin and eosin.

Instrumentation.

Force curves were acquired with a Nanoscope IIIa Multimode scanning probe microscope (Veeco Instruments Inc., Santa Barbara, Calif.). The samples were transferred to the AFM without drying and placed in a commercially available liquid cell (Veeco Instruments Inc.). Silicon nitride cantilevers with a borosilicate sphere as the tip (1 μm radius, Novascan Technologies, Inc. Ames, Iowa) were used to sample a large area of the trabecular meshwork. The nominal spring constant of the cantilevers was 0.06 N/m. Force curves were obtained on at least 10 different locations, at either random locations on the sample or in a line with each measurement location separated by approximately 50 μm. Data exhibiting non-linear behavior or a large adhesion with the surface were not included in the analysis and a minimum of three locations were used to calculate a mean elastic modulus value. In addition, when data was acquired at random locations a minimum of three force curves were obtained at each location. Each force curve was taken at a rate of 2 μm/sec.

Data Analysis.

The force curves were analyzed using the Hertz model for a sphere in contact with a flat surface using the Punias force curve analysis software. To obtain an accurate modulus value, the optical sensitivity and the spring constant of each cantilever was determined. Optical sensitivity was measured as the slope of the force curve, taken in PBS, when the tip was in contact with a rigid surface. The optical sensitivity was used to convert cantilever deflection in volts to deflection in nanometers (x). Spring constants (k) were measured using Sader's method (Sader, et al., Rev Sci Instrum (1995) 66:4583-4587). The force was determined by F=kx. The Hertz model provides a relationship between the loading force and the indentation, which for a spherical indenter is:

$$F = \frac{4}{3} \frac{E {( \sqrt{R} )}^{3 / 2}}{1 {-}^{’ 2}} \qquad ( 1 )$$

where F is the loading force in Newtons, ν is Poisson's ratio (assumed to be 0.5), δ is the indentation depth, E is the elastic modulus in Pascals and R is the radius of the tip. The values obtained from the force curve are z, zₒ, d and dₒ, where z is the piezo displacement, d is the cantilever deflection, and zₒ and dₒ are the values at initial contact of the tip with the sample. These values can be used to calculate the indentation, which is given by:

δ=(z−zₒ)−(d−dₒ) (2)

Using these equations and knowing that F=k(d−dₒ), where k is the cantilever spring constant, gives the following equation for E:

$$E = \frac{3}{4} \frac{k ( d - {d}_{o} ) ( 1 - {v}^{2} )}{\sqrt{R} {( ( z - {z}_{o} ) - ( d - {d}_{o} ) )}^{3 / 2}} \qquad ( 3 )$$

Models to Describe Flow.

In the combined biomechanical and biofluidic model, the HTM is considered as an elastic porous membrane (36 mm in length, 0.3 mm in width and 50 μm in thickness) with parallel cylindrical micropores embedded (with the original porous diameter of 1 μm and the porosity of 350/mm², chosen to be within the range reported in the literature, e.g., Ethier, et al., Invest Ophthalmol Vis Sci 1998; 39:2041-2048). The IOP causes the HTM to bow outward, which leads to the enlargement of the pore size. The size of micropore is a function of the elastic modulus of HTM (E) and aqueous outflow rate (Q), which can be derived from the following equations. Taking the HTM as a thin elastic porous membrane, the assumption of thin plate with small deflections is applied, in which the normal stresses transverse to the plate (HTM) are disregarded (Timoshenko, et al., Theory of plates and shells, 2nd Edition. New York: McGraw-Hill; 1987). Under the external pressure difference, the HTM (of length 1, width w, and thickness t) deforms into a spherical shape with a central angle of 2α. The deflection of HTM membrane can be related to the IOP (ΔP) acting on the HTM and the elastic modulus. According to Laplace equation, the relationship among IOP, geometrical parameters and material properties of the HTM can be expressed as:

$$Δ P = T ( \frac{sin α}{l / 2} + \frac{sin α}{w / 2} ) = 2 T sin α ( \frac{1}{l} + \frac{1}{w} ) \approx \frac{2 T sin α}{w} \qquad ( 4 )$$

where T represents internal tension parallel to the HTM and the length of the HTM is much greater than the width. Furthermore, the internal tension can be derived from the strain-stress relationship, which leads to the following governing equation:

$$\frac{E}{1 - {γ}^{2}} ( \frac{α}{sin α} - 1 ) = \frac{w Δ P}{2 t sin α} \qquad ( 5 )$$

where γ is Poisson's ratio. As can be seen, the central angle is a function of the IOP and the elastic modulus of the HTM. Eventually, the overall dynamic fluidic resistance of the HTM membrane can be calculated as follows:

$$R = \frac{IOP}{Q} = \frac{8 μ t}{NA π {r}^{4}} = \frac{8 π μ t}{{{NAA}_{c} ( r , α )}^{2}} \qquad ( 6 )$$

where the aqueous outflow rate (Q) ranges from 2-3 μL/min and μ is the aqueous viscosity. Other structural parameters include the overall area and thickness of JCT (A and t), the porous density (N), as well as the area of an individual micropore (A_{c}), which can be calculated from the original pore size and the central angle of the HTM. Thus, the “effective” flow pathway leads to t/NA or 0.013 μm as modeled herein.

HTM Cell Line and Fluorescent Staining Reagents.

Primary HTM cells were obtained from corneal buttons from donors with no ocular diseases and that were not suitable for transplant. HTM cells were isolated as previously described (Rhee, et al., Experimental Eye Research 77:749-746), and cultured in DMEM/F12 (HyClone, Fisher Scientific, Waltham, Mass.) medium with 10% fetal bovine serum (Atlanta Biologicals, Lawrenceville, Ga. New primary cultures were incubated for four days with 0.1 μM dexamethasone, and only cells that had increased expression of myocilin (a marker for HTM cell identity) were used. Experiments were conducted using cells before the 7th passage. Normal HTM cells were employed because growth of HTM cells from glaucomatous individuals is generally not robust enough for cell culture experiments (Tschumper, et al., (1990) Investigative Ophthalmology & Visual Science 31:1327-1331; Caballero, et al., (2003) Biochemical and Biophysical Research Communications 308:346-352; Schutt, et al., (2008) Klinische Monatsblatter Fur Augenheilkunde 225:548-554; and Gasiorowski, et al., (2009) Exp Eye Res 88:671-675). Cells were plated onto glass surfaces at 100,000 cells per surface. A similar number of cells were plated onto the biomimetic PA hydrogels. The cells were allowed to attach to the surfaces for 12 hours prior to the start of the experiment. Fluorescent images of the actin cytoskeleton were obtained from fixed cells stained with phalloidin-AlexaFluor 568 (Invitrogen, Carlsbad, Calif.). Fluorescent images of the cell membrane were obtained from live cells stained with wheat germ agglutinin-AlexaFluor 488 (Invitrogen).

Fabrication of Substrates:

Hydrogels have been fabricated and protocols have been established to make surfaces with a range of compliance. Hydrogels that provide for compliances of around 3, 50 and 100 kPa to model the compliances of normal (3 kPa), transitional (50 kPa) and glaucomatous (100 kPa) HTM and hard surfaces (GPa) similar to standard lab plasticware are used. Polyacrylamide, polyethylene glycol and patterned hydrogels can be used.

Biomimetic Polyacrylamide Hydrogel Synthesis.

The compliance of the trabecular meshwork was measured using atomic force microscopy (Russell, et al., (2010) “Compliance of the human trabecular meshwork: implications about glaucoma,” In The Association for Research in Vision and Ophthalmology, Fort Lauderdale, Fla.). The average Young's modulus of the tissue was 4.0±2.2 kPa for normal tissue and 80.0±32.5 kPa for glaucomatous tissue. Based on these results, two different polyacrylamide gels (PA) gels that mimicked the compliant conditions of normal (homeomimetic) and glaucomatous (pathomimetic) tissue were prepared. Polyacrylamide gels, formed by free-radical polymerization, have been widely used in the study of substrate compliance on cell behavior because the compliance can be easily tuned by altering the cross-linker density (Pelham, et al., (1997) Proc Natl Acad Sci USA 94:13661-13665, Discher, et al., (2005) Science 310:1139-1143). In addition, the surfaces of the PA gels can be functionalized to ensure cell adhesion and survival (Pelham, et al., supra).

Pre-gel monomer solutions were prepared in 50 ml conical tubes. For the homeomimetic tissue polyacrylamide hydrogels (HPA), 1.1 ml of a pre-mixed solution of acrylamide (Am) and N,N′-methylenebisacrylamide (BIS) (Am:BIS 29:1, 40%, Fisher Scientific), and 400 μl of (3 acrylamidepropyl)trimethylammonium chloride (API, 75% w/w, Sigma Aldrich, St. Louis, Mo.) were dissolved in 8.5 ml of ultrapure water (Millipore, Billerica, Mass.). To create pathomimetic substrates that approximate the compliance of glaucomatous meshwork (PPA), an Am:BIS solution (18:1) was prepared by dissolving 3.554 g of Am (MP Biomedical, Solon, Ohio), 192.7 mg of BIS (Sigma), and 400 μl of API in 9.6 ml of ultrapure water. Dissolved oxygen is known to interfere with this type of free radical polymerization. The above solutions were kept at room temperature for one hour, prior to the addition of polymerization initiator and catalyst, to obtain repeatable results. Once equilibrated, 200 μl of 10% w/v solution of ammonium persulfate (Fisher Scientific) and 30 μl of tetramethylethylenediamine (Sigma) were added. These solutions were gently swirled in the conical tubes for 10-15 seconds and quickly poured into empty Criterion gel casting cassettes (1 mm thick, Bio-Rad, Hercules, Calif.). These containers are closed to the atmosphere except for a thin strip at the top of the cassette. After 60 minutes, the cassettes were cracked open, and substrates were cut using a ⅜ inch diameter round punch. The top centimeter of gel, closest to atmosphere was discarded due to inconsistency in compliance values with the rest of the gel. To remove unreacted reagents, the cut PA gels were placed in polystyrene dishes and rinsed three times in 1×PBS (HyClone, Fisher Scientific). The gels were then sterilized in PBS with short wavelength (280 nm) UV light for 30 minutes. Finally, the PBS was replaced with fresh, sterile PBS and the substrates were stored in a CO2 incubator at 37° C. for at least 24 hours to attain their final equilibrium swelling.

The PA gels were then adhered to UV-cleaned, glass-bottom petri dishes (World Precision Instruments, Sarasota, Fla.), incubated in HTM medium for 12 hours, and then coated with a mixture of fibronectin and collagen (FNC coating mix, Athena Environmental Sciences, Baltimore, Md.) for 10 minutes prior to cell seeding. HTM cells were added to these dishes in media and were allowed to incubate overnight to ensure proper cell adhesion to the FNC-coated PA gels. Immediately prior to AFM measurements, the HTM media was rinsed away with PBS. This was done because fetal bovine serum is known to inactivate Lat-B (Spector, et al., (1989) Cell Motil Cytoskeleton 13:127-144).

Contact Mechanics.

The contact mechanics of the PA substrates and the HTM cells were studied with two MFP-3D atomic force microscopes (Asylum Research, Santa Barbara, Calif.) interfaced with either an Olympus (FluoView 1000 laser scanning confocal microscope, Olympus America, Center Valley, Pa.) or Zeiss (Axio Observer A1, Carl Zeiss, Thornwood, N.Y.) inverted microscope. No differences in contact mechanics of the samples were observed between the two instruments. The AFM probes in the substrate studies were silicon nitride cantilevers (PNP-TR-50, k=60 pN/nm, NanoAndMore, Lady's Island, S.C.) with a square pyramid tip incorporated at the free end. For each experiment, the actual spring constant of the cantilever was determined by monitoring the amplitude of the lever's thermal vibration at resonance and applying the equipartition theorem (Hutter, et al., (1993) Review of Scientific Instruments 64:3342-3342). Probe indentation measurements were obtained over the central region of the cell where the nucleus was present. Phase contrast imaging demonstrates alignment.

Compliance of the HTM cells and PA gels was quantified by fitting the force, generated by the indenting probe, vs. the depth of indentation with equation 1 (Hertz, H. (1882) J. Reine Angew. Mathematik 92:156-171; Love, A. E. H. (1939) Quarterly Journal of Mathematics 10:161-175; Harding, et al., (1945) Proceedings of the cambridge philosophical society 41:16-26).

$$F = \frac{2}{π} tan ( α ) \frac{E}{1 - {v}^{2}} {δ}^{2} \qquad Eq . 1$$

Where α is the half-angle opening of the square pyramid tip (35°), ν is Poisson's ratio, E is Young's modulus and δ is the penetration depth. By using Eq. 1, we have assumed that the HTM cells and PA gels were perfectly elastic, isotropic, and infinitely thick and that the square pyramid indenter was a rigid cone. A Poisson ratio of 0.5 (incompressible) for both the cell and polyacrylamide gels was assigned, since both samples were well hydrated. This allows a solution for Young's modulus of the sample.

Application of Eq. 1 is not straightforward, as both the cells and gels were not perfectly elastic or isotropic, they were viscoelastic and anisotropic, and they were also not infinitely thick. However, in the limit of small indentations both the cells and gels were well described by Eq. 1. The method proposed by Mahaffey et al., (Phys Rev Lett (2000) 85:880-883) was applied to quantitatively define the indentation depth over which these viscoelastic materials behave as elastic bodies. This is accomplished by plotting experimental values of E vs. δ during tip indentation and noting the penetration depth over which E remains constant. In addition to defining the region of elastic response, defining the exact position of contact between the probe and cell or gel can introduce error in determining E. To help minimize this, very weak cantilever springs with manufacturers' spring constants of 60 pN/nm were used. Finally, many curves were averaged together to produce a mean and standard deviation force vs. indentation plots. The main graph shows 25 different indentation vs. force curves (grey dots) measured at multiple positions on a PA gel. Contact was defined by visually noting where the cantilever deviates from a forward extrapolation of zero deflection. From these curves we generated a single curve of the average and two additional curves of the positive and negative standard deviations (black line with error bars, and open circles in the inset). A linear least-squares fit of the data using Eq. 1 (black lines) was then applied to the elastic region of these three curves to give an average and standard deviation of Young's modulus.

For each cell experiment, 5-7 cells were probed at ˜2 μm/s, with 5 indentation curves on each cell. Drive speeds were approximate due to the fact that the free end of cantilever slowed down once contact was made and the probe began to indent the sample. Young's modulus of the HTM cells was initially measured before Lat-B exposure. Force measurements were conducted in PBS. The cells were then exposed to a 0.2 μM Lat-B solution for 30 minutes. The concentration of Lat-B was based on previous studies examining the effects of Lat-B on IOP (Ethier, et al., (2006) Invest Ophthalmol Vis Sci 47:1991-1998). At the end of this dose period, the Lat-B was rinsed away and the cells recovered in HTM medium. Young's modulus data were measured at 90 and 270 minutes (in PBS) post removal of Lat-B. Experiments were done in triplicate using primary HTM cells from three different donors.

Fluorescence Microscopy:

HTM cells are incubated and then fixed in 4% paraformaldehyde in PBS for 20 minutes to check the localization and expression of cytoskeletal proteins. After washing with phosphate buffered saline, the cells are permeabilized with 0.5% Triton-X for 5 minutes and then washed again. Primary and fluorescent secondary antibodies are added serially at this point. TRITC labeled phalloidin and DAPI are used to fluorescently stain the actin filaments and the cell nucleus, respectively. Images of the cells on the substrates are taken with an Axiovert 200M microscope with deconvolution capabilities and analyzed using the Zeiss KS300 software program. Immunofluorescence is done for cytoskeletal proteins (actin, vinculin, paxillin and focal adhesion kinase) at 24, 48, and 72 hours, and at 1 and 2 weeks using the standard protocols¹⁴⁰.

Real Time PCR:

mRNA is extracted from cells after they have been grown on substrates for three days and one week. This time period was chosen so that parallel cultures, either untreated or treated with either 10⁻⁷M dexamethasone or 4×10⁻¹¹M activated TGF-β, could be harvested to assess any possible interaction of biophysical and biochemical cues on gene expression. The mRNA is extracted with Qiagen RNeasy kits following the manufacturer's protocol. The amount of mRNA is measured and 75 ng is used with the one step Taqman kit for real time PCR. Individual reactions with the Step One real time PCR machine is performed with a total volume of 10 μl. The reverse transcription reaction is done for 30 minutes at 50° C. followed by PCR enzyme activation for 10 minutes at 95° C. Forty cycles of 60° C. for 1 minute followed by 95° C. for 15 seconds is done. The reference mRNA is the 18S ribosomal RNA. At least three reactions are run for each sample. This procedure has been demonstrated to successfully obtain a relative quantitative analysis of mRNAs in HTM cells⁸⁶.

Western Blot:

Cells grown on surfaces treated with either dexamethasone or TGF-β are lysed at the third day, 1 and 2-week time points with M-Per buffer containing 0.002% protease inhibitor cocktail. Protein concentration is assessed using the Bradford protein assay. Samples of 10 μg are electrophoresed on a 10% polyacrylamide gel and the protein transferred to nitrocellulose membranes as previously described¹⁴¹. Antibodies for the proteins being detected are commercially available or publicly described. A chemiluminescent detection method is used with peroxidase linked antibodies to mouse, chick or rabbit in conjunction with the LumiGLO chemiluminesent kit. The banding pattern obtained on the x-ray film is quantified using ImageQuant software with the actin band serving as a normalized reference for protein loading. Imaging can also be done using a CCD camera without the use of x-ray film. These protocols are standard in the laboratory and have been used successfully in the past.

Zymography:

For the analysis of active MMPs, zymography is used. The change in activity of the MMPs is examined both with changes in biophysical cues (compliance and topography) and the combination of biophysical and biochemical cues (dexamethasone and TGF-β, test agents). Time points for measurements are the same as described in the above sections (24, 48, and 72 hours and 1 and 2 weeks). HTM cells are homogenized at 4° C. in 50 mM Tris-HCl, pH 7.4, including a protease inhibitor cocktail to eliminate nonspecific protease activity and centrifuged at 15,000 rpm, 4° C. Protein concentrations for each tissue sample are measured, and fifteen micrograms of protein from each sample are mixed with non-reducing sample buffer containing SDS. Samples are loaded onto SDS polyacrylamide gels containing either gelatin as a substrate for MMP-2 or β-casein as a substrate for MMP-3. After electrophoresis, gels are rinsed in Triton X-100 to remove SDS and incubated overnight at 37° C. in development buffer. After incubation, gels are stained with 0.1% Coomassie blue and destained until bands of gel digestion are visible. Digested bands are compared with active-recombinant human MMP-2 and MMP-3 standards included as positive controls. MMP-2 and MMP-3 bands are verified by Western blot analysis. Multiple replicates are performed for each sample, and the densitometry readings of band intensities are averaged.

Phagocytosis:

Since phagocytosis is thought to play a role in cellular remodeling, alterations in biophysical cues may change phagocytic rates. With both dexamethasone and TGF-β treatments, phagocytosis is decreased. Phagocytosis is measured using the pHrodo BioParticles Kit from Invitrogen. The bioparticles are not fluorescent extracellularly; however, they fluoresce when phagocytosed. Cells are imaged using a fluorescence microscope (Zeiss Axiovert 200) to show that the bioparticles are being phagocytosed by the HTM cells. For quantification, levels of phagocytosis in HTM cells are assayed in a 96-well format using a fluorescent microplate reader. Polymeric substrates are fabricated on 3 mm glass cover slips (Belco) and inserted into wells of 96 well plates.

### Example 2

### Determination of the Compliance for the HTM in Normal and Glaucomatous Eyes and how Alterations in Substratum Compliance Influence HTM Cells

Histologic examination of donor corneal buttons confirmed the presence of HTM and Schlemm's canal tissue in the examined buttons. Tissue removed via sharp dissection was largely ciliary body muscle and pigmented uvea. Isolated HTM was difficult to precisely orient for histology, but the samples were confirmed to be composed predominantly of HTM beams and cells, with variable, but typically small amounts of scleral collagen, rare melanocytes and, in one case, remnants of Descemet's membrane. The inner wall of Schlemm's canal was multifocally/segmentally absent from post-isolation corneal buttons. Trabecular meshwork tissue was identified in well oriented regions of isolated HTM tissue, and the inner wall of Schlemm's canal was identified in some sections.

Cytoskeletal Changes with Changes in Substratum Compliance.

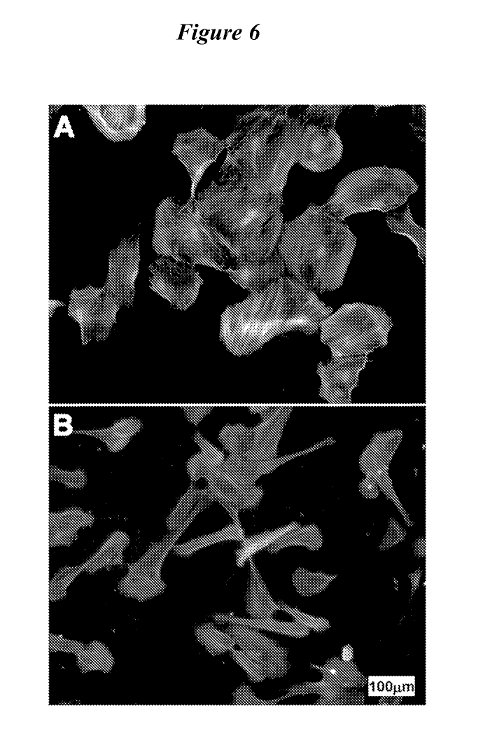

Actin cytoskeleton of HTM cells were investigated, either grown on a polyacrylamide gel with a modulus of 3 kPa or on glass, which has a modulus in the 50 GPa range (FIG. 6). On the glass surface (A), the HTM cells are spread out and contain multiple stress fibers. On the more compliant gel (B), the cells are more rounded with far fewer stress fibers. Also, HTM cells have a longer population doubling time of 36.6 hours on the 3 kPa gels compared to around 28 hours on the tissue culture polystyrene (“TCPS”). This is consistent with the conclusion that compliance does influence cellular behaviors.

Compliance of the HTM is Decreased (Becomes Stiffer) in Glaucomatous Globes.

Compliance of HTM was measured using AFM. AFM force curves, a plot of cantilever deflection vs. z piezo movement, were obtained on HTM from normal and glaucomatous donors. The force curves obtained on these tissues, taken from the internal surface of Schlemm's canal, had the same characteristics as those obtained on other soft, elastic materials (Cappella, et al., Surf Sci Rep (1999) 34:1-104; and Clifford and Seah, Appl Surf Sci (2005) 252:1915-1933). In general, the force curves consist of a straight-line approach when the tip is still away from the surface. As the tip comes into contact with the surface there is a gradual increase in the deflection of the cantilever, as expected for soft samples. Large “pull-off” forces were not typically observed as the tip was retracted from the surface, indicating negligible adhesion of the tip with the HTM. The approach and retract curves overlapped, indicating an absence of viscoelastic effects at the indentation rate used (1 Hz).

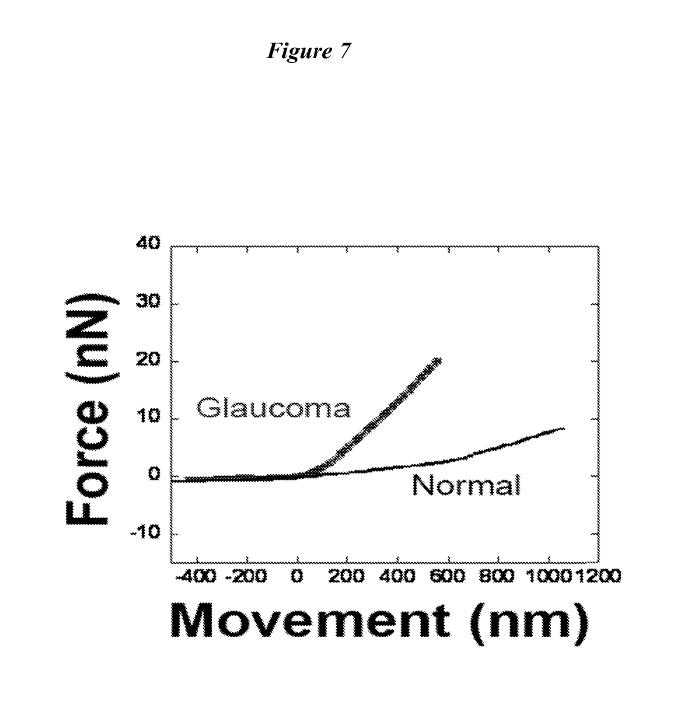

FIG. 7 shows the force curves generated for HTM from normal donor tissue (black) and glaucomatous tissue (blue). In this figure, the tip is approaching the sample on the left and then engages the HTM generating a slope. The modulus for the normal HTM (78 year-old) was 6.5 kPa. At least six normal meshworks have been measured. The average value for the six meshworks is 3.1 kPa with the range from 1.7 to 6.5 kPa. In contrast, the meshwork from the glaucomatous donor had a much lower compliance. The 92 year-old donor with glaucoma was diagnosed eight years prior to death and had been taking Travaprost and Alphagan. The slope of this curve is much steeper than that of the normal 78 year-old indicating the glaucomatous HTM is more than 10 fold stiffer (less compliant) than normal HTM. The force curves document the glaucomatous HTM has a modulus of 75 kPa. The average value of six glaucomatous meshworks is 108.7 kPa.

Compliance from Glaucomatous HTM Varies Spatially.

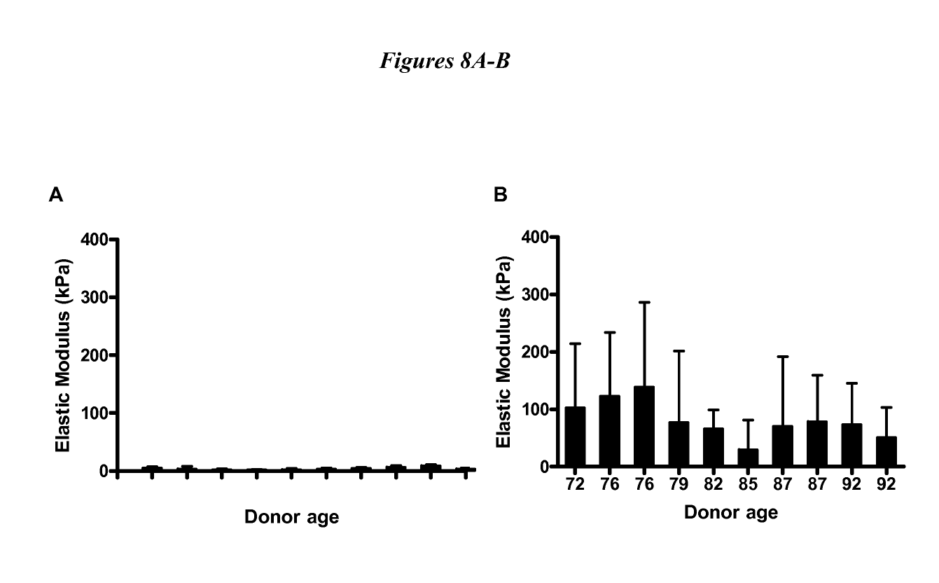

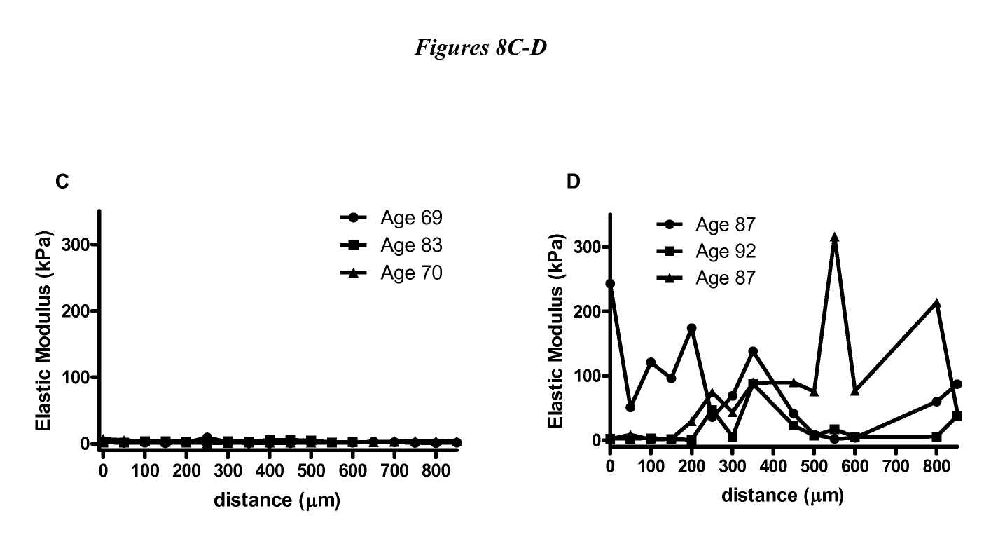

Force curves taken at 50 micron distances along HTM from normal subjects show little variation of modulus values (FIGS. 8A-B). Measurement of the elastic modulus in normal HTM (n=10, mean age 65 years, range 32-83 years) ranged from 1.7 kPa to 8.8 kPa (mean 4.0±2.2 kPa) (FIG. 8A). In comparison, glaucomatous HTM (n=10, mean age 84 years, range 72-92 years) had a significant increase in elastic modulus ranging from 29.6 kPa to 138.4 kPa (mean 80.8±32.5 kPa, p<0.0001) (FIG. 8B). Analysis of 50 μm intervals along the glaucomatous HTM revealed large variations ranging from less than 10 kPa to greater than 200 kPa (FIG. 8D). In contrast, minimal variations in elastic modulus were identified in normal HTM, with all values less than 15 kPa (FIG. 8C). The variation observed in TM tissue from glaucomatous patients is consistent with segmental flow in diseased HTM, and indicates that the stiffening of the HTM is not uniform around the HTM. This result also suggests the disease process is not uniform around the whole tissue. These regions of increased rigidity are becoming less active, in a turnover sense, and thus become less sensitive to the mechanical stretch that signals the need to adjust IOP and less able to respond as well. Force curves on other glaucomatous HTM also showed a similar segmental variation.

Table 2 delineates the average results obtained with both normal and glaucomatous samples. Force curves taken on six normal and six glaucomatous HTM samples show the marked difference in compliance between the two sets of samples regardless of age.

TABLE 2
Average Modulus Values for Normal and Glaucomatous HTMs.

| Normal | | Glaucoma | |
|---|---|---|---|
| | Young's Modulus | | Young's Modulus |
| Age | (kPA) | Age | (kPA) |
| 39 | 3.4 | 72 | 102.6 |
| 56 | 1.9 | 79 | 180 |
| 61 | 1.7 | 82 | 73.2 |
| 77 | 4.0 | 83 | 74 |
| 78 | 6.5 | 87 | 134.5 |
| 83 | 3.5 | 92 | 88 |

A mathematical model was employed to determine if the change in modulus between normal and glaucomatous HTM might influence facility of aqueous outflow. A mathematical model has been established to evaluate the impact of modulating intrinsic stiffness on outflow resistance. In this model, the HTM is considered as an elastic porous membrane with conjugated biomechanical and biofluidic responses. FIG. 19 shows that the increase in HTM stiffness increases the flow resistance of the JCT. This increase is due to a pressure elevation across the JCT/SC layer that enlarges the pore size, which is inversely proportional to the 4^{th} power of the outflow resistance. As a consequence, the pathologically increased stiffness of the HTM in glaucomatous eyes leads to considerably higher outflow resistance than that of the normal control group. The model shows a similar trend of the outflow facility dependence on IOP/flow rate variation, as predicted by previous biomechanical models (Ethier, Exp Eye Res (2002) 74:161-172). While this model is one of several that could be applied, in all conventional models a change in facility would be substantially influenced by the changes in elastic modulus measured between the normal and diseased HTM.

Intrinsic Compliance of HTM Cells is Modulated by the Compliance of the Underlying Substratum.

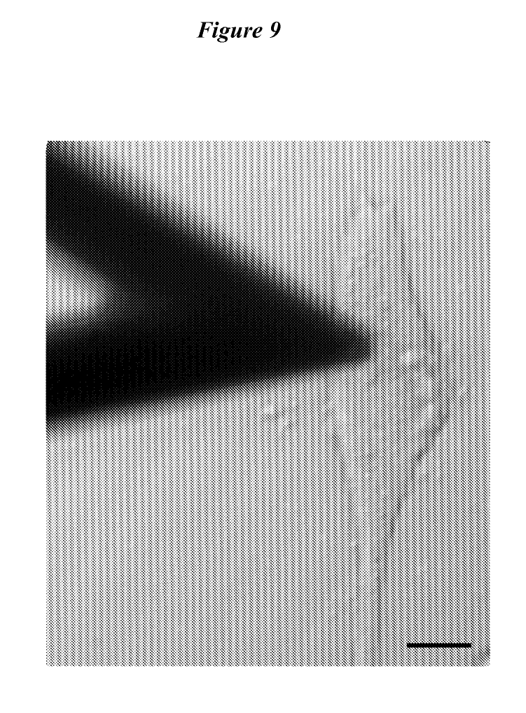

FIG. 9 shows the tip of the Asylum model MFP-3D AFM with the Nikon phase configuration in contact with a single HTM cell. This cell was from a 33 year-old donor. The image highlights the fact that the HTM cells on nanoscale topography elongate and align with the patterned surfaces in the biomimetic range. In this case, the surface was a 1200 nm pitch substrate (pitch=ridge width plus groove width), the groove and ridges can be clearly seen in this photograph. AFM data on cultured HTM cells revealed a five-fold change in compliance of the cells grown on polyacrylamide substrates with a modulus similar to the native HTM compared to cells grown on rigid glass (Table 3). The moduli of HTM cells from the same donor button were measured by AFM on either glass or a polyacrylamide gel. As the compliance of the surface decreases, the cell itself also becomes more rigid.

TABLE 3
The Intrinsic Modulus of HTM Cells is Dependent
on the Modulus of the Underlying Substratum

| | | Modulus of | Modulus of HTM |
|---|---|---|---|
| Surface | Type of Surface | Surface | Cells on Surface |
| glass | Hard | 50 GPa | 5.5 kPa |
| Polyacrylamide | Soft—similar to | 3 kPa | 1.1 kPa |
| hydrogel | normal HTM | | |

These data indicate that cells modulate their compliance in response to the compliance of the underlying substrate. As the compliance of the substrate decreases (became stiffer), the overlying cells respond by decreasing their intrinsic compliance. These data are consistent with the conclusion that the compliance of the HTM cells are altered by the compliance of the extracellular matrix or synthetic substrata that they interface with. As the compliance of the ECM decreases in glaucoma, the cells will modify their cytoskeleton to increase their moduli and thus decrease their compliance. The more rigid HTM cells will, in turn, decrease outflow facility.

Although the HTM cells are synthesizing ECM, at least over a six day time period, the modulus value of the HTM cells were not dramatically influenced by new ECM proteins. The modulus of HTM cells measured by the Asylum AFM on day 1 after plating was 1.9±1.4 kPa. On the sixth day, the modulus was 2.1±1.1 kPa.

Biophysical Cues Modulate the Effects of Certain Compounds that Influence Cytoskeleton.

Latrunculin B influences cytoskeleton of cells by associating with actin. This compound has been suggested as a therapeutic to increase outflow by changing cellular shape. FIG. 10 shows the effect of 0.2 μM latrunculin on HTM cells in as little as 30 minutes. The micrographs were taken with a confocal AFM. The live HTM cells were labeled with fluorescent wheat germ agglutinin and the latrunculin was added. In 30 minutes the cells were starting to round up. By one hour, the cells were completely rounded. Measurements of compliance were taken prior to the introduction of drug, and at the 30 minute time point. The cells had moduli of 2.0 kPa prior to addition of latrunculin, and 0.07 kPa at 30 minutes. The concentration of drug used was not in the range of 1-100 μM used in other published studies. It should be noted, however, that the higher ranges (100 μM) were topically applied to primate eyes (20 μl) in one bolus while our studies had the drug present during the entire one hour time period. The effects of the latrunculin were profound.

Cell Morphology and Response to Exposure and Recovery from Latrunculin B.

The morphology of HTM cells adhered to glass and the glaucomatous (pathomimetic)polyacrylamide gel (PPA, 92.2±10.4 kPa) substrates were predominantly elongated with pronounced asymmetry. On the homeomimetic polyacrylamide gel (HPA, 4.0±1.5 kPa), the cells were predominantly adhered in a radially symmetric fashion and were considerably rounder in appearance (See, FIGS. 6 and 10). When exposed to Lat-B, HTM cells on glass and PPA substrates responded in a similar fashion to other cell types previously studied (Spector, et al., (1983) Science 219:493-495, Wakatsuki, et al., (2001) Journal of Cell Science 114:1025-1036, Coue, et al., (1987) Febs Letters 213:316-318; Cha, et al., (2004) J Cell Sci 117:3353-3365). Lat-B disrupted the dynamic process of actin filament maintenance and the actin polymerization process rapidly shifted towards net depolymerization. Confocal imaging showed this resulted in a dramatic change in cell membrane morphology, demonstrating that actin filaments play a central role in HTM cell morphology. Confocal fluorescent emission images obtained before and at 30 minutes of exposure to Lat-B on glass by staining live cells with wheat germ agglutinin (a lectin that adheres to the cell membrane) revealed that as the cell retracted during drug treatment, it adhered to the glass with delicate processes. Z-stack images showed that these processes helped to anchor the retracting cell to the underlying glass. These strands resembled retraction fibers observed during mitosis (Sanger, et al., (1984) Cell Tissue Res 237:409-417). Similar cable-like filaments have also been reported by Evanko, et. al., (J Histochem Cytochem (2009) 57:1041-1060). A significant number of cells detached from the glass and GPA substrates during subsequent rinsing steps, suggesting these were very weak adhesion points.

After rinsing the Lat-B from the dish, the cells very quickly began to repolymerize actin filaments, and they returned to their pre-exposure morphology within 60-90 minutes. The dramatic changes in cell morphology observed on glass and PPA substrates were not as pronounced for HTM cells adhered to HPA substrates as a result of exposure to, or recovery from Lat-B.

Effect of Substrate Modulus on Cell Modulus Before and During Recovery from Latrunculin B.

Prior to treatment with Lat-B, HTM cell compliance was proportional to the compliance of the underlying substrate. On glass with GPa modulus, Young's modulus of the HTM cells was 2.7±0.7 kPa. The elastic modulus of HTM cells grown on pathomimetic substrates was 1.8±0.5 kPa and was approximately 12% higher than cells grown on homeomimetic substrates 1.6±0.2 kPa (Table 4). The differences between cells on pathomimetic and homeomimetic surfaces were also present, and significantly more pronounced, upon recovery from Lat-B exposure. Fluorescent images of the actin cytoskeleton confirmed that HTM cells on glass and the PPA substrates produced significantly more actin stress fibers than cells adhered to the more compliant HPA substrate. The greater degree of actin polymerization contributed to increased cell rigidity as demonstrated by an increased Young's modulus. The fluorescent images also demonstrated HTM cells on the HPA substrates were more rounded in appearance and had fewer actin stress fibers. Loss of stress fibers with Lat-B exposure did not alter cell morphology as noticeably as the cells on the other two substrates.

The modulus of HTM cells at 30 minutes of Lat-B exposure was difficult to obtain, since the contracted cells offered almost no resistance to the indenting probe and the initial point of contact could not be reliably determined. However, when compared to pre-exposure levels, the cell resistance to deformation at 30 minutes of Lat-B exposure was substantially decreased. During recovery, HTM cell modulus initially increased and then decreased back to pre-exposure levels by 270 minutes. For example, on glass, Young's modulus of the HTM cells before drug exposure was 2.7±0.7 kPa (circles). At 90 minutes after removal of Lat-B, the cell modulus increased to 11.0±2.3 kPa (squares) and then decreased back to 2.3±0.2 kPa (triangles) by 270 minutes of recovery. A similar trend was observed for both the PPA and HPA substrates. The difference in cell elastic modulus from before exposure and at 90 minutes of recovery was less as the substrate compliance increased. The trend of increasing cell elastic modulus with decreasing substrate compliance was found at all time points, pre and post exposure to Lat-B, with a maximum percentage difference of approximately 54% (PPA to HPA) at 90 minutes of recovery. The combined results for HTM cells on glass and the compliant substrates mimicking glaucomatous and healthy tissue are shown in Table 4. Table 4 shows HTM Cell modulus before exposure to Latrunculin B, and 90 and 270 minutes after exposure and removal of Latrunculin B. Cell modulus on both glass and pathomimetic substrate was significantly increased at 90 minutes of actin re-polymerization. Within 270 minutes, cells have returned to pre-dose modulus values.

TABLE 4
HTM Cell Modulus on Substrates (kPa)

| Condition | Glass¹ | Pathomimetic² | Homeomimetic³ |
|---|---|---|---|
| Pre-dose | 2.7 ± 0.7 | 1.8 ± 0.5 | 1.6 ± 0.2 |
| 90 min. rec. | 11.0 ± 2.3 | 4.0 ± 1.8 | 2.3 ± 0.2 |
| 270 min. rec. | 2.3 ± 0.2 | 2.0 ± 0.4 | 1.7 ± 0.5 |

¹Elastic modulus, E, in GPa range;
²E = 92 ± 10 kPa;
³E = 4.0 ± 1.5 kPa

### Example 3

### Determination of how Biophysical Properties Modulate HTM-Mediated ECM Protein Production

Biophysical Cues Alter Gene Expression.

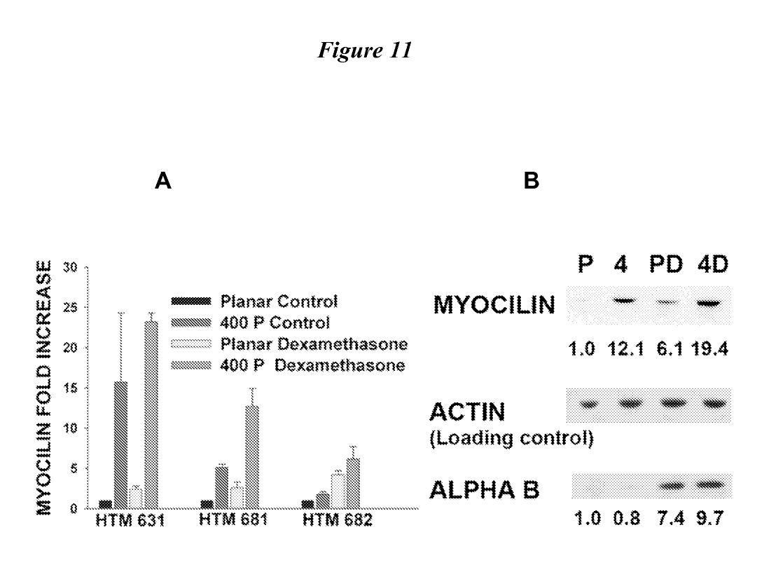

Biophysical cues alter not only cellular behaviors, but they also change gene and protein expression levels. In HTM cells grown on either flat surfaces or chemically identical 400 nm pitch surfaces of equal compliance, the mRNA for myocilin is increased significantly on the topographically patterned surfaces containing features in the biomimetic range compared to the flat surfaces. The myocilin mRNA is further increased if the HTM cells are incubated with dexamethasone. The three HTM cultures presented on the left side of FIG. 11 were from different donors. The increases in mRNA caused increases in protein expression as well. For the HTM 631 cells, the western blots for the loading control (actin) and myocilin demonstrate a marked increase in myocilin expression for cells cultured on topographically patterned surfaces compared to chemically identical flat surfaces of equal compliance (FIG. 11 right). The increases in myocilin protein expression (19-fold observed for cells cultured on the 400 nm pitch surfaces with dexamethasone) match the increases seen with the mRNA levels.

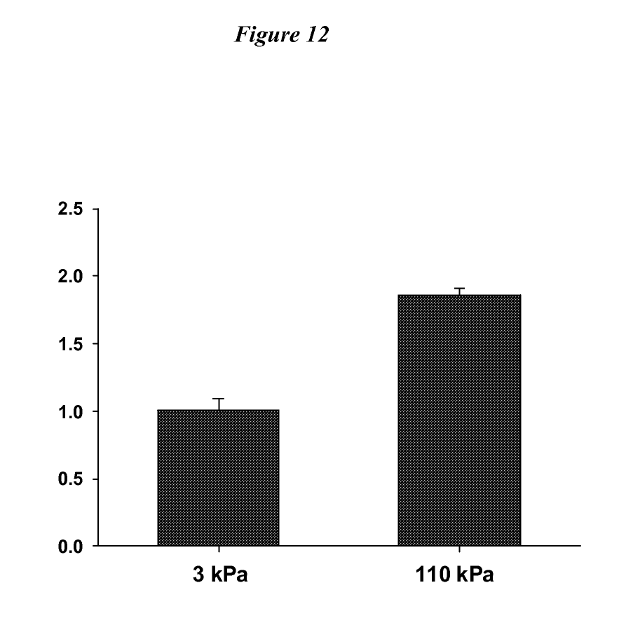

On polyacrylamide hydrogels, the level of gene expression of transglutaminase-2 showed an increase in cells grown on gels of 100 kPa compared to cells on 3 kPa substrates (FIG. 12). The levels of transglutaminase were 1.8 fold higher on the more rigid substrate consistent with the reported increases in this enzyme in glaucomatous HTM. Transglutaminase has been shown to bind to collagen and fibronectin and cause crosslinks in these two ECM proteins which would cause the combination to become more rigid.

Biophysical Cues Influence Gene Splicing.

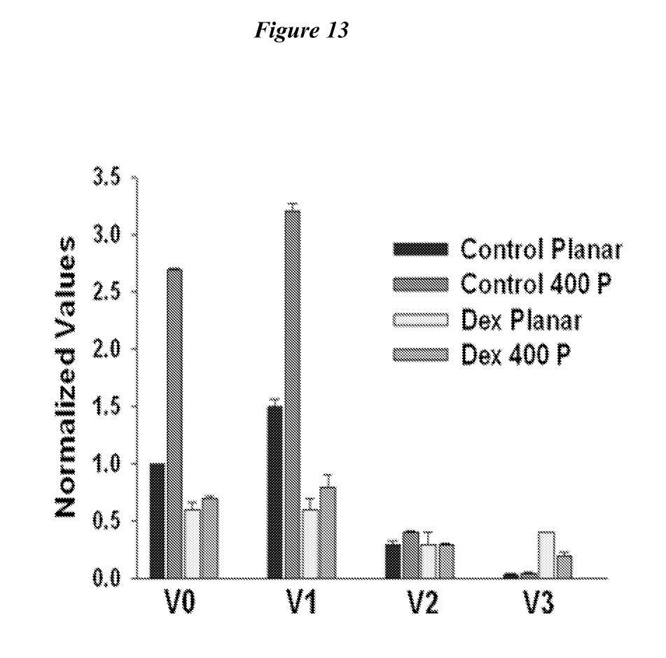

Substrates having topographic features of biomimetic size scale also influence gene splicing. Versican isoform expression is modulated by the presence of topographic cues. In this case, both the V0 and V1 isoforms, the two isoforms with the highest level of chondroitin sulfate binding sites, have increased expression on the patterned surfaces (FIG. 13). The V2 and V3 isoforms were not changed on these substrates. Curiously, the addition of dexamethasone negated the effects of the biophysical cues and no changes in isoform expression were observed with the V0 and V1 forms. These results indicate cells can respond to biochemical and biophysical cues in a variety of ways and that sometimes one set of cues will oppose the other. These data also indicate that glycoaminoglycan deposition can be influenced by biophysical cues.

Deposition of Fibronectin by HTM Cells Increases with Increasing Stiffness of the Substratum.

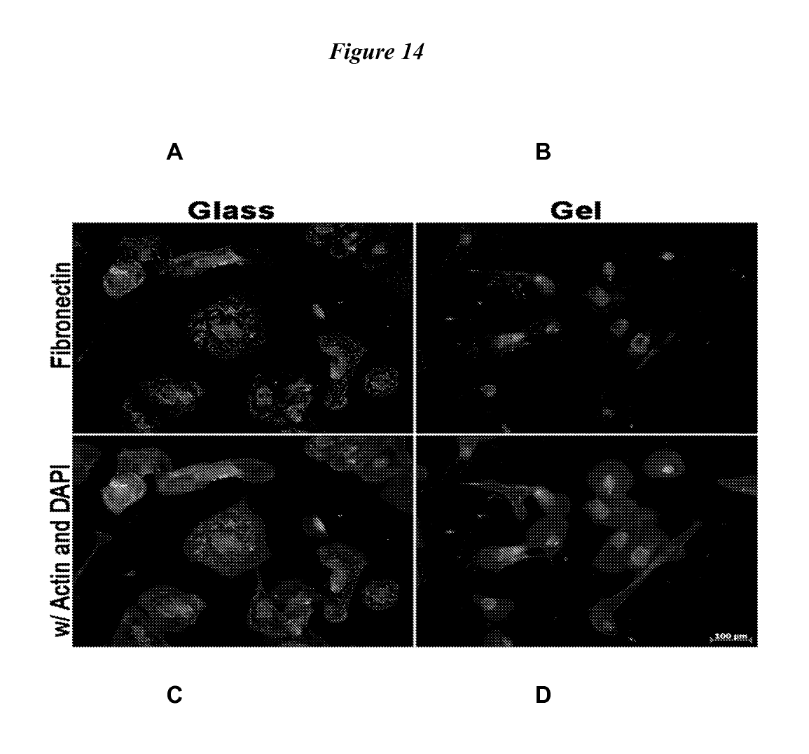

Data on fibronectin expression indicates that growth of cells on compliant polyacrylamide gels decreased expression to 37% of the value of cells grown on plastic. The addition of dexamethasone on these compliant hydrogels brought the expression to 1.2 fold the level seen with the cells on plastic (not treated with dexamethasone). The increase in fibronectin seen with increasing stiffness of the substratum is consistent with finding glaucomatous cells exhibiting increased fibronectin expression^{134,135}. Further support for increased expression of fibronectin on more rigid substrates is shown in FIG. 14. The top two panels show immunofluorescent staining of the fibronectin expressed by HTM cells on glass or a polyacrylamide gel with a modulus of 30 kPa. There was a marked increase in the staining on the less compliant surface as had been expected based on the mRNA data. The bottom figures show the fibronectin staining (green), phalloidin staining for actin (red) and the DAPI staining of the nuclei (blue). The data are consistent with the conclusion that glaucomatous HTM cells, interacting with a stiffer substratum, exhibit an increase in secretion of elements of the ECM (increased fibronectin) with decreased remodeling activity (decrease MMP expression).

### Example 4

### Determination how Biophysical Properties of the Substratum Modulate HTM Mediated ECM Remodeling

MMPs are Present in HTM Cells.

MMPs are present in the HTM and are involved with ECM remodeling. To verify, the expression of the mRNA levels of the MMPs by real time PCR was investigated and the results showed that MMP-2 and MMP-14 had the highest level of expression in HTM cells (Table 5).

TABLE 5

| Class of MMP | MMP | Expression Level |
|---|---|---|
| Collagenase | MMP-1 | ++ |
| Gelatinases | MMP-2 | +++++ |
| | MMP-9 | ND |
| Stromelysins | MMP-3 | ++ |
| | MMP-11 | ++ |
| MT-MMPs | MMP-14 | +++++ |
| | MMP-15 | +++ |
| | MMP-16 | +++ |
| | MMP-17 | ++ |
| | MMP-24 | +++ |
| Other MMPs | MMP-12 | + |
| | MMP-19 | +++ |

Comparisons among the MMPs were made by using the threshold values (Ct) of samples done in triplicate. Those MMPs with the lowest Ct values were given an expression level of 5+ and those with higher Ct values were given lesser expression levels. MMP-9 was not detected in any of our samples. The size of the PCR amplified fragment was verified in each case. The reporting on all the MMPs in the HTM cells is published¹³⁶.

Decreasing Substratum Compliance Causes a Decrease in MMP Gene Expression.

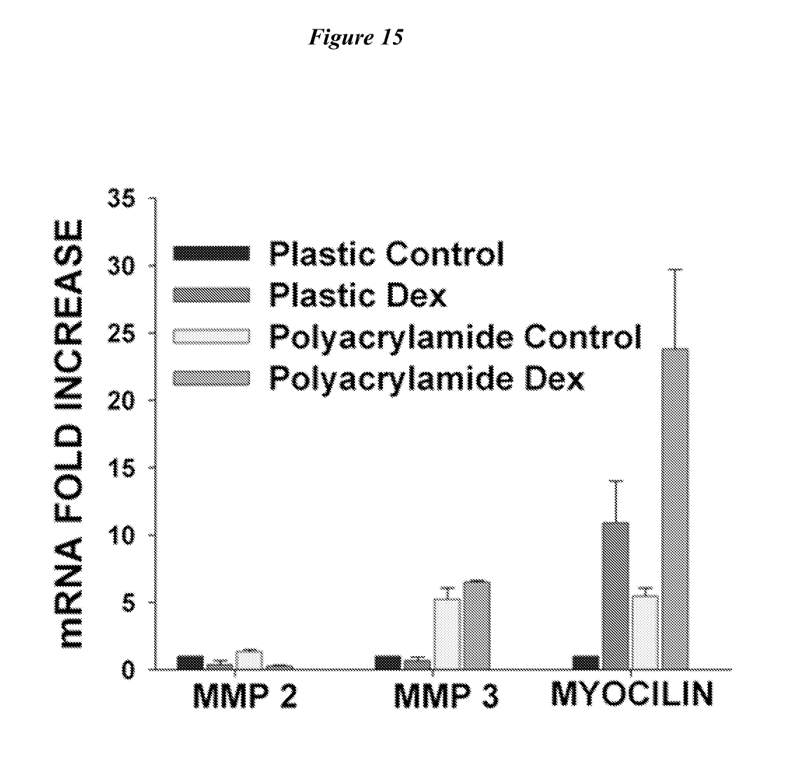

Similar to the biophysical cues from topographic surfaces, changes in the compliance of the substrate upon which the cells are cultured influences gene expression. Data with HTM cells grown on compliant 30 kPa polyacrylamide surfaces compared to stiff plastic surfaces (3 GPa) revealed that the compliance of the substrate made a difference to both MMP-3 and myocilin mRNA expression (FIG. 15). With both MMP-3 and myocilin there were substantial increased in gene expression (about 5-fold in each case) when the cells were grown on the polyacrylamide rather than on the hard tissue culture plastic. The increase in MMP-3 is consistent with increased remodeling activity occurring on the more compliant substrates. Myocilin has been reported to have increased expression on more compliant but uncharacterized surfaces⁸⁷. The data are consistent with the conclusion that biophysical cues are inducing the expression levels of HTM cell myocilin to levels observed in vivo. The addition of dexamethasone further increased the mRNA levels, consistent with the data obtained from organ cultured HTMs. While the addition of dexamethasone had no effect on the expression of the MMP-3, cells cultured on compliant polyacrylamide had about 6-fold more expression than those cells cultured on plastic. The growth of the cells on the polyacrylamide surfaces had no effect on the expression of MMP-2, but addition of dexamethasone decreased expression by around a factor of 3 on both surfaces.

HTM Cells are Phagocytic.

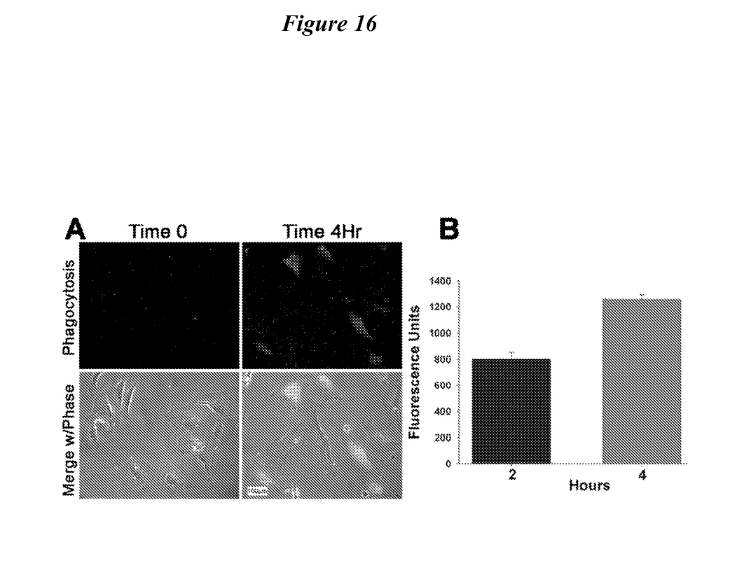

ECM remodeling was also investigated in relation to phagocytosis. Active phagocytosis was observed with the HTM cells (FIG. 16). After a four hour challenge with the pHrodo BioParticles, numerous fluorescent particles were taken up and localized inside the HTM cells. Control cells did not take up the particles. A time course for uptake of these particles was undertaken. In the right part of this figure is a time course of uptake of these particles by HTM cells as measured in a fluorescent plate reader. The four hour time point was significantly higher in fluorescence (and therefore with higher cellular uptake of the particles) than the two hour time point. These data document the ability to quantify phagocytic activity.

Fabrication of Nanotopography.

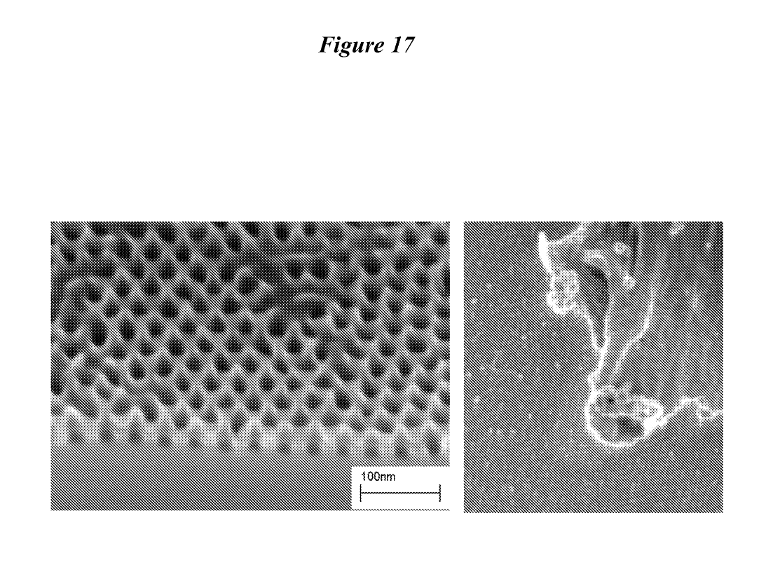

Nanotypography can be provided by anisotropic ridges and grooves, as well as pores of a similar size scale. It has been shown that the size scale and not the shape of the features that impacts cell behavior^{130,121}. FIG. 17 shows a fabricated substrate patterned with pores of 30 nm. Polymeric patterned surfaces with pores of varying feature dimensions can be fabricated. This surface pattern provides an isotropically ordered surface (surface order more characteristic of that seen in native basement membrane) and will compliment our studies using anisotropically ordered patterns of ridges and grooves.

### REFERENCES

1. Quigley, H. A. and Broman, A. T. The number of people with glaucoma worldwide in 2010 and 2020. Br J Ophthalmol. 2006; 90:262-267.
2. The AGIS Investigators. The Advanced Glaucoma Intervention Study (AGIS): 7. the relationship between control of intraocular pressure and visual field deterioration. Am J Ophthalmol. 2000; 130:429-440.
3. Collaborative Normal-Tension Glaucoma Study Group. Comparison of glaucomatous progression between untreated patients with normal-tension glaucoma and patients with therapeutically reduced intraocular pressures. Am J Ophthalmol. 1998; 126:487-497.
4. Heijl A, Leske M C, Bengtsson B, Hyman L, Bengtsson B, and Hussein M. For the Early Manifest Glaucoma Trial Group: reduction of intraocular pressure and glaucoma progression. Arch Ophthalmol. 2002; 120:1268-1279.
5. Bill, A. Some aspects of aqueous humour drainage. Eye. 1993; 7 (Pt 1):14-19.
6. Johnstone M A and Grant W M. Pressure-dependent changes in structure of the aqueous outflow system of human and monkey eyes. Am J Ophthalmol. 2008; 75:365-383.
7. Johnson, M. C. and Kamm, R. D. The role of Schlemm's canal in aqueous outflow from the human eye. Invest Ophthalmol Vis Sci 1983 March; 24(3):3. 1983; 24:3-5.
8. Millar, C and Kaufman, P. L. Aqueous humor: secretion and dynamics. 1987; 5-44.
9. Nerenberg, P. S., Salsas-Escat, R., and Stultz, C. M. Collagen—a necessary accomplice in the metastatic process. Cancer Genomics Proteomics. 2007; 4:319-328.
10. Goldbarg, S. H., Elmariah, S., Miller, M. A., and Fuster, V. Insights into degenerative aortic valve disease. J Am Coll Cardiol. 9-25-2007; 50:1205-1213.
11. Burns, W. C., Kantharidis, P., and Thomas, M. C. The role of tubular epithelial-mesenchymal transition in progressive kidney disease. Cells Tissues Organs. 2007; 185:222-231.
12. Fuchshofer, R., Welge-Lussen, U., and Lutjen-Drecoll, E. The effect of TGF-beta2 on human trabecular meshwork extracellular proteolytic system. Exp Eye Res. 2003; 77:757-765.
13. Acott, T. S. and Kelley, M. J. Extracellular matrix in the trabecular meshwork. Exp Eye Res. 2008; 86:543-561.
14. Johnson, M. ‘What controls aqueous humour outflow resistance?’. Exp Eye Res. 2006; 82:545-557.
15. Ethier, C. R., Kamm, R. D., Palaszewski, B. A., Johnson, M. C., and Richardson, T. M. Calculations of flow resistance in the juxtacanalicular meshwork. Invest Ophthalmol Vis Sci. 1986; 27:1741-1750.
16. Acott, T. S., Kingsley, P. D., Samples, J. R., and Van Buskirk, E. M. Human trabecular meshwork organ culture: morphology and glycosaminoglycan synthesis. Invest Ophthalmol Vis Sci. 1988; 29:90-100.
17. Knepper, P. A., Goossens, W., Hvizd, M., and Palmberg, P. F. Glycosaminoglycans of the human trabecular meshwork in primary open-angle glaucoma. Invest Ophthalmol Vis Sci. 1996; 37:1360-1367.
18. Zhao, X. and Russell, P. Versican splice variants in human trabecular meshwork and ciliary muscle. Mol Vis 2005 Aug. 12. 1911; 603-608.
19. Tane, N., Dhar, S., Roy, S., Pinheiro, A., Ohira, A., and Roy, S. Effect of excess synthesis of extracellular matrix components by trabecular meshwork cells: possible consequence on aqueous outflow. Exp Eye Res. 2007; 84:832-842.
20. Tamm, E. R. and Fuchshofer, R. What increases outflow resistance in primary open-angle glaucoma? Surv Ophthalmol. 2007; 52 Suppl 2:S101-S104.
21. Tovar-Vidales, T., Roque, R., Clark, A. F., and Wordinger, R. J. Tissue transglutaminase expression and activity in normal and glaucomatous human trabecular meshwork cells and tissues. Invest Ophthalmol Vis Sci. 2008; 49:622-628.
22. Xue, W., Wallin, R., Olmsted-Davis, E. A., and Borras, T. Matrix GLA protein function in human trabecular meshwork cells: inhibition of BMP2-induced calcification process. Invest Ophthalmol Vis Sci. 2006; 47:997-1007.
23. Xue, W., Comes, N., and Borras, T. Presence of an established calcification marker in trabecular meshwork tissue of glaucoma donors. Invest Ophthalmol Vis Sci. 2007; 48:3184-3194.
24. Rohen, J. W., Lutjen-Drecoll, E., Flugel, C., Meyer, M., and Grierson, I. Ultrastructure of the trabecular meshwork in untreated cases of primary open-angle glaucoma (POAG). Exp Eye Res. 1993; 56:683-692.
25. Lutjen-Drecoll, E. Morphological changes in glaucomatous eyes and the role of TGFbeta2 for the pathogenesis of the disease. Exp Eye Res. 2005; 81:1-4.
26. Gottanka, J., Johnson, D. H., Martus, P., and Lutjen-Drecoll, E. Severity of optic nerve damage in eyes with POAG is correlated with changes in the trabecular meshwork. J Glaucoma. 1997; 6:123-132.
27. Rhee, D. J., Fariss, R. N., Brekken, R., Sage, E. H., and Russell, P. The matricellular protein SPARC is expressed in human trabecular meshwork. Exp Eye Res. 2003; 77:601-607.
28. Peters, D. M., Herbert, K., Biddick, B., and Peterson, J. A. Myocilin binding to Hep II domain of fibronectin inhibits cell spreading and incorporation of paxillin into focal adhesions. Exp Cell Res. 2-15-2005; 303:218-228.
29. Wentz-Hunter, K., Kubota, R., Shen, X., and Yue, B. Y. Extracellular myocilin affects activity of human trabecular meshwork cells. J Cell Physiol. 2004; 200:45-52.
30. Shen, X., Koga, T., Park, B. C., SundarRaj, N., and Yue, B. Y. Rho GTPase and cAMP/protein kinase A signaling mediates myocilin-induced alterations in cultured human trabecular meshwork cells. J Biol Chem. 1-4-2008; 283:603-612.
31. Polansky, J. R., Fauss, D. J., Chen, P., Chen, H., Lutjen-Drecoll, E., Johnson, D., Kurtz, R. M., Ma, Z. D., Bloom, E., and Nguyen, T. D. Cellular pharmacology and molecular biology of the trabecular meshwork inducible glucocorticoid response gene product. Ophthalmologica. 1997; 211:126-139.
32. Russell, P., Tamm, E. R., Grehn, F. J., Picht, G., and Johnson, M. The presence and properties of myocilin in the aqueous humor. Invest Ophthalmol Vis Sci. 2001; 42:983-986.
33. Karali, A., Russell, P., Stefani, F. H., and Tamm, E. R. Localization of myocilin/trabecular meshwork—inducible glucocorticoid response protein in the human eye. Invest Ophthalmol Vis Sci. 2000; 41:729-740.
34. Zillig, M., Wurm, A., Grehn, F. J., Russell, P., and Tamm, E. R. Overexpression and properties of wild-type and Tyr437H is mutated myocilin in the eyes of transgenic mice. Invest Ophthalmol Vis Sci. 2005; 46:223-234.
35. Bradley, J. M., Kelley, M. J., Zhu, X., Anderssohn, A. M., Alexander, J. P., and Acott, T. S. Effects of mechanical stretching on trabecular matrix metalloproteinases. Invest Ophthalmol Vis Sci 2001 June 1988; 1505-1513.
36. Ronkko, S., Rekonen, P., Kaarniranta, K., Puustjarvi, T., Terasvirta, M., and Uusitalo, H. Matrix metalloproteinases and their inhibitors in the chamber angle of normal eyes and patients with primary open-angle glaucoma and exfoliation glaucoma. Graefes Arch Clin Exp Ophthalmol. 2007; 245:697-704.
37. Bradley, J. M., Kelley, M. J., Zhu, X., Anderssohn, A. M., Alexander, J. P., and Acott, T. S. Effects of mechanical stretching on trabecular matrix metalloproteinases. Invest Ophthalmol Vis Sci. 2001; 42:1505-1513.
38. WuDunn, D. The effect of mechanical strain on matrix metalloproteinase production by bovine trabecular meshwork cells. Curr Eye Res. 2001; 22:394-397.
39. Johnson, D. H., Richardson, T. M., and Epstein, D. L. Trabecular meshwork recovery after phagocytic challenge. Curr Eye Res. 1989; 8:1121-1130.
40. Zhou, L., Fukuchi, T., Kawa, J. E., Higginbotham, E. J., and Yue, B. Y. Loss of cell-matrix cohesiveness after phagocytosis by trabecular meshwork cells. Invest Ophthalmol Vis Sci. 1995; 36:787-795.
41. Buller, C., Johnson, D. H., and Tschumper, R. C. Human trabecular meshwork phagocytosis. Observations in an organ culture system. Invest Ophthalmol Vis Sci. 1990; 31:2156-2163.
42. Zhang, X., Ognibene, C. M., Clark, A. F., and Yorio, T. Dexamethasone inhibition of trabecular meshwork cell phagocytosis and its modulation by glucocorticoid receptor beta. Exp Eye Res. 2007; 84:275-284.
43. Park, C. H. and Latina, M. A. Effects of gamma-interferon on human trabecular meshwork cell phagocytosis. Invest Ophthalmol Vis Sci. 1993; 34:2228-2236.
44. Coppolino, M. G., Dierckman, R., Loijens, J., Collins, R. F., Pouladi, M., Jongstra-Bilen, J., Schreiber, A. D., Trimble, W. S., Anderson, R., and Grinstein, S. Inhibition of phosphatidylinositol-4-phosphate 5-kinase Ialpha impairs localized actin remodeling and suppresses phagocytosis. J Biol Chem. 11-15-2002; 277:43849-43857.
45. Tschumper, R. C. and Johnson, D. H. Trabecular meshwork cellularity. Differences between fellow eyes. Invest Ophthalmol Vis Sci. 1990; 31:1327-1331.
46. Caballero, M., Liton, P. B., Epstein, D. L., and Gonzalez, P. Proteasome inhibition by chronic oxidative stress in human trabecular meshwork cells. Biochem Biophys Res Commun. 8-22-2003; 308:346-352.
47. Gasiorowski, J. Z. and Russell, P. Biological properties of trabecular meshwork cells. Exp Eye Res. 8-26-2008;
48. Yu, A. L., Fuchshofer, R., Kampik, A., and Welge-Lussen, U. Effects of oxidative stress in trabecular meshwork cells are reduced by prostaglandin analogues. Invest Ophthalmol Vis Sci. 2008; 49:4872-4880.
49. Polansky, J. R., Fauss, D. J., Chen, P., Chen, H., Lutjen-Drecoll, E., Johnson, D., Kurtz, R. M., Ma, Z. D., Bloom, E., and Nguyen, T. D. Cellular pharmacology and molecular biology of the trabecular meshwork inducible glucocorticoid response gene product. Ophthalmologica. 1997; 211:126-139.
50. Polansky, J. R., Fauss, D. J., and Zimmerman, C. C. Regulation of TIGR/MYOC gene expression in human trabecular meshwork cells. Eye. 2000; 14:503-514.
51. Tamm, E. R., Russell, P., Epstein, D. L., Johnson, D. H., and Piatigorsky, J. Modulation of myocilin/TIGR expression in human trabecular meshwork.
52. Ishibashi, T., Takagi, Y., Mori, K., Naruse, S., Nishino, H., Yue, B. Y., and Kinoshita, S. cDNA microarray analysis of gene expression changes induced by dexamethasone in cultured human trabecular meshwork cells. Invest Ophthalmol Vis Sci. 2002; 43:3691-3697.
53. Lo, W. R., Rowlette, L. L., Caballero, M., Yang, P., Hernandez, M. R., and Borras, T. Tissue differential microarray analysis of dexamethasone induction reveals potential mechanisms of steroid glaucoma. Invest Ophthalmol Vis Sci. 2003; 44:473-485.
54. Steely, H. T., Browder, S. L., Julian, M. B., Miggans, S. T., Wilson, K. L., and Clark, A. F. The effects of dexamethasone on fibronectin expression in cultured human trabecular meshwork cells. Invest Ophthalmol Vis Sci. 1992; 33:2242-2250.
55. Clark, A. F., Wilson, K., McCartney, M. D., Miggans, S. T., Kunkle, M., and Howe, W. Glucocorticoid-induced formation of cross-linked actin networks in cultured human trabecular meshwork cells. Invest Ophthalmol Vis Sci. 1994; 35:281-294.
56. Clark, A. F., Brotchie, D., Read, A. T., Hellberg, P., English-Wright, S., Pang, I. H., Ethier, C. R., and Grierson, I. Dexamethasone alters F-actin architecture and promotes cross-linked actin network formation in human trabecular meshwork tissue. Cell Motil Cytoskeleton. 2005; 60:83-95.
57. Read, A. T., Chan, D. W., and Ethier, C. R. Actin structure in the outflow tract of normal and glaucomatous eyes. Exp Eye Res. 2007; 84:214-226.
58. Gonzalez, P., Epstein, D. L., and Borras, T. Genes upregulated in the human trabecular meshwork in response to elevated intraocular pressure. Invest Ophthalmol Vis Sci. 2000; 41:352-361.
59. Tumminia, S. J., Mitton, K. P., Arora, J., Zelenka, P., Epstein, D. L., and Russell, P. Mechanical stretch alters the actin cytoskeletal network and signal transduction in human trabecular meshwork cells. Invest Ophthalmol Vis Sci. 1998; 39:1361-1371.
60. Keller, K. E., Kelley, M. J., and Acott, T. S. Extracellular matrix gene alternative splicing by trabecular meshwork cells in response to mechanical stretching. Invest Ophthalmol Vis Sci. 2007; 48:1164-1172.
61. Wordinger, R. J., Agarwal, R., Talati, M., Fuller, J., Lambert, W., and Clark, A. F. Expression of bone morphogenetic proteins (BMP), BMP receptors, and BMP associated proteins in human trabecular meshwork and optic nerve head cells and tissues. Mol Vis. 7-15-2002; 8:241-250.
62. Wordinger, R. J., Fleenor, D. L., Hellberg, P. E., Pang, I. H., Tovar, T. O., Zode, G. S., Fuller, J. A., and Clark, A. F. Effects of TGF-beta2, BMP-4, and gremlin in the trabecular meshwork: implications for glaucoma. Invest Ophthalmol Vis Sci. 2007; 48:1191-1200.
63. Wordinger, R. J., Zode, G., and Clark, A. F. Focus on Molecules: Gremlin. Exp Eye Res. 12-4-2007;
64. Picht, G., Welge-Luessen, U., Grehn, F., and Lutjen-Drecoll, E. Transforming growth factor beta 2 levels in the aqueous humor in different types of glaucoma and the relation to filtering bleb development. Graefes Arch Clin Exp Ophthalmol. 2001; 239:199-207.
65. Zhao, X., Ramsey, K. E., Stephan, D. A., and Russell, P. Gene and Protein Expression Changes in Human Trabecular Meshwork Cells Treated with Transforming Growth Factor-{beta}. Invest Ophthalmol Vis Sci. 2004; 45:4023-4034.
66. Welge-Lussen, U., May, C. A., Eichhorn, M., Bloemendal, H., and Lutjen-Drecoll, E. AlphaB-crystallin in the trabecular meshwork is inducible by transforming growth factor-beta. Invest Ophthalmol Vis Sci. 1999; 40:2235-2241.
67. Welge-Lussen, U., May, C. A., and Lutjen-Drecoll, E. Induction of tissue transglutaminase in the trabecular meshwork by TGF-beta1 and TGF-beta2. Invest Ophthalmol Vis Sci. 2000; 41:2229-2238.
68. Flugel-Koch, C., Ohlmann, A., Fuchshofer, R., Welge-Lussen, U., and Tamm, E. R. Thrombospondin-1 in the trabecular meshwork: localization in normal and glaucomatous eyes, and induction by TGF-beta1 and dexamethasone in vitro. Exp Eye Res. 2004; 79:649-663.
69. Wang, N., Chintala, S. K., Fini, M. E., and Schuman, J. S. Activation of a tissue-specific stress response in the aqueous outflow pathway of the eye defines the glaucoma disease phenotype. Nat Med. 2001; 7:304-309.
70. Hosseini, M., Rose, A. Y., Song, K., Bohan, C., Alexander, J. P., Kelley, M. J., and Acott, T. S. IL-1 and TNF induction of matrix metalloproteinase-3 by c-Jun N-terminal kinase in trabecular meshwork. Invest Ophthalmol Vis Sci. 2006; 47:1469-1476.
71. Kelley, M. J., Rose, A. Y., Song, K., Chen, Y., Bradley, J. M., Rookhuizen, D., and Acott, T. S. Synergism of TNF and IL-1 in the induction of matrix metalloproteinase-3 in trabecular meshwork. Invest Ophthalmol Vis Sci. 2007; 48:2634-2643.
72. Fleenor, D. L., Pang, I. H., and Clark, A. F. Involvement of AP-1 in interleukin-1alpha-stimulated MMP-3 expression in human trabecular meshwork cells. Invest Ophthalmol Vis Sci. 2003; 44:3494-3501.
73. Tamm, E. R., Russell, P., Johnson, D. H., and Piatigorsky, J. Human and monkey trabecular meshwork accumulate alpha B-crystallin in response to heat shock and oxidative stress. Invest Ophthalmol Vis Sci 1996 November 1937; 2402-2413.
74. Li, G., Luna, C., Liton, P. B., Navarro, I., Epstein, D. L., and Gonzalez, P. Sustained stress response after oxidative stress in trabecular meshwork cells. Mol Vis. 2007; 13:2282-2288.
75. Johnson, D. H. Human trabecular meshwork cell survival is dependent on perfusion rate. Invest Ophthalmol Vis Sci 1996 May; 37(6):1. 1996; 37:1-8.
76. Kiland, J. A., Gabelt, B. T., and Kaufman, P. L. Studies on the mechanism of action of timolol and on the effects of suppression and redirection of aqueous flow on outflow facility. Exp Eye Res. 2004; 78:639-651.
77. Ramos, R. F. and Stamer, W. D. Effects of cyclic intraocular pressure on conventional outflow facility. Invest Ophthalmol Vis Sci. 2008; 49:275-281.
78. Crowston, J. G. and Weinreb, R. N. Glaucoma medication and aqueous humor dynamics. Curr Opin Ophthalmol. 2005; 16:94-100.
79. Kaufman, P. L. Enhancing trabecular outflow by disrupting the actin cytoskeleton, increasing uveoscleral outflow with prostaglandins, and understanding the pathophysiology of presbyopia interrogating Mother Nature: asking why, asking how, recognizing the signs, following the trail. Exp Eye Res. 2008; 86:3-17.
80. Rao, P. V., Deng, P. F., Kumar, J., and Epstein, D. L. Modulation of aqueous humor outflow facility by the Rho kinase-specific inhibitor Y-27632. Invest Ophthalmol Vis Sci. 2001; 42:1029-1037.
81. Rao, P. V., Deng, P., Sasaki, Y., and Epstein, D. L. Regulation of myosin light chain phosphorylation in the trabecular meshwork: role in aqueous humour outflow facility. Exp Eye Res. 2005; 80:197-206.
82. Ethier, C. R., Read, A. T., and Chan, D. W. Effects of latrunculin-B on outflow facility and trabecular meshwork structure in human eyes. Invest Ophthalmol Vis Sci. 2006; 47:1991-1998.
83. Okka, M., Tian, B., and Kaufman, P. L. Effects of latrunculin B on outflow facility, intraocular pressure, corneal thickness, and miotic and accommodative responses to pilocarpine in monkeys. Trans Am Ophthalmol Soc. 2004; 102:251-257.
84. Peyton, S. R. and Putnam, A. J. Extracellular matrix rigidity governs smooth muscle cell motility in a biphasic fashion. J Cell Physiol. 2005; 204:198-209.
85. Peyton, S. R., Ghajar, C. M., Khatiwala, C. B., and Putnam, A. J. The emergence of ECM mechanics and cytoskeletal tension as important regulators of cell function. Cell Biochem Biophys. 2007; 47:300-320.
86. Russell, P., Gasiorowski, J. Z., Nealy, P. F., and Murphy, C. J. Response of human trabecular meshwork cells to topographic cues on the nanoscale level. Invest Ophthalmol Vis Sci. 2008; 49:629-635.
87. Schlunck, G., Han, H., Wecker, T., Kampik, D., Meyer-Ter-Vehn, T., and Grehn, F. Substrate rigidity modulates cell matrix interactions and protein expression in human trabecular meshwork cells. Invest Ophthalmol Vis Sci. 2008; 49:262-269.
88. Foley, J. D., Grunwald, E. W., Nealey, P. F., and Murphy, C. J. Cooperative modulation of neuritogenesis by PC12 cells by topography and nerve growth factor. Biomaterials. 2005; 26:3639-3644.
89. Pelham, R. J., Jr. and Wang, Y. L. Cell locomotion and focal adhesions are regulated by the mechanical properties of the substrate. Biol Bull. 1998; 194:348-349.
90. Discher, D. E., Janmey, P., and Wang, Y. L. Tissue cells feel and respond to the stiffness of their substrate. Science. 11-18-2005; 310:1139-1143.
91. Yeung, T., Georges, P. C., Flanagan, L. A., Marg, B., Ortiz, M., Funaki, M., Zahir, N., Ming, W., Weaver, V., and Janmey, P. A. Effects of substrate stiffness on cell morphology, cytoskeletal structure, and adhesion. Cell Motil Cytoskeleton. 2005; 60:24-34.
92. McDaniel, D. P., Shaw, G. A., Elliott, J. T., Bhadriraju, K., Meuse, C., Chung, K. H., and Plant, A. L. The stiffness of collagen fibrils influences vascular smooth muscle cell phenotype. Biophys J. 3-1-2007; 92:1759-1769.
93. Khatiwala, C. B., Peyton, S. R., and Putnam, A. J. Intrinsic mechanical properties of the extracellular matrix affect the behavior of pre-osteoblastic MC3T3-E1 cells. Am J Physiol Cell Physiol. 2006; 290:C1640-C1650.
94. Janmey, P. A. and McCulloch, C. A. Cell mechanics: integrating cell responses to mechanical stimuli. Annu Rev Biomed Eng. 2007; 9:1-34.
95. Solon, J., Levental, I., Sengupta, K., Georges, P. C., and Janmey, P. A. Fibroblast adaptation and stiffness matching to soft elastic substrates. Biophys J. 12-15-2007; 93:4453-4461.
96. Wang, N., Tolic-Norrelykke, I. M., Chen, J., Mijailovich, S. M., Butler, J. P., Fredberg, J. J., and Stamenovic, D. Cell prestress. I. Stiffness and prestress are closely associated in adherent contractile cells. Am J Physiol Cell Physiol. 2002; 282:C606-C616.
97. Stamenovic, D., Mijailovich, S. M., Tolic-Norrelykke, I. M., Chen, J., and Wang, N. Cell prestress. II. Contribution of microtubules. Am J Physiol Cell Physiol. 2002; 282:C617-C624.
98. Khatiwala, C. B., Peyton, S. R., Metzke, M., and Putnam, A. J. The regulation of osteogenesis by ECM rigidity in MC3T3-E1 cells requires MAPK activation. J Cell Physiol. 2007; 211:661-672.
99. Heling, A., Zimmermann, R., Kostin, S., Maeno, Y., Hein, S., Devaux, B., Bauer, E., Klovekorn, W. P., Schlepper, M., Schaper, W., and Schaper, J. Increased expression of cytoskeletal, linkage, and extracellular proteins in failing human myocardium. Circ Res. 4-28-2000; 86:846-853.
100. Wang, X., LeMaire, S. A., Chen, L., Shen, Y. H., Gan, Y., Bartsch, H., Carter, S. A., Utama, B., Ou, H., Coselli, J. S., and Wang, X. L. Increased collagen deposition and elevated expression of connective tissue growth factor in human thoracic aortic dissection. Circulation. 7-4-2006; 114:I200-I205.
101. Chiarelli, F., Trotta, D., Verrotti, A., and Mohn, A. Treatment of hypertension and microalbuminuria in children and adolescents with type 1 diabetes mellitus. Pediatr Diabetes. 2002; 3:113-124.
102. Schrot, S., Weidenfeller, C., Schaffer, T. E., Robenek, H., and Galla, H. J. Influence of hydrocortisone on the mechanical properties of the cerebral endothelium in vitro. Biophys J. 2005; 89:3904-3910.
103. Pelham, R. J., Jr. and Wang, Y. High resolution detection of mechanical forces exerted by locomoting fibroblasts on the substrate. Mol Biol Cell. 1999; 10:935-945.
104. Yu, T. and Ober, C. K. Methods for the topographical patterning and patterned surface modification of hydrogels based on hydroxyethyl methacrylate. Biomacromolecules. 2003; 4:1126-1131.
105. Peppas, N. A., Huang, Y., Torres-Lugo, M., Ward, J. H., and Zhang, J. Physicochemical foundations and structural design of hydrogels in medicine and biology. Annu Rev Biomed Eng. 2000; 2:9-29.
106. Engler, A. J., Griffin, M. A., Sen, S., Bonnemann, C. G., Sweeney, H. L., and Discher, D. E. Myotubes differentiate optimally on substrates with tissue-like stiffness: pathological implications for soft or stiff microenvironments. J Cell Biol. 9-13-2004; 166:877-887.
107. Abrams, G. A., Goodman, S. L., Nealey, P. F., Franco, M., and Murphy, C. J. Nanoscale topography of the basement membrane underlying the corneal epithelium of the rhesus macaque. Cell Tissue Res. 2000; 299:39-46.
108. Abrams, G. A., Murphy, C. J., Wang, Z. Y., Nealey, P. F., and Bjorling, D. E. Ultrastructural basement membrane topography of the bladder epithelium. Urol Res. 2003; 31:341-346.
109. Abrams, G. A., Schaus, S. S., Goodman, S. L., Nealey, P. F., and Murphy, C. J. Nanoscale topography of the corneal epithelial basement membrane and Descemet's membrane of the human. Cornea. 2000; 19:57-64.
110. Brody, S., Anilkumar, T., Liliensiek, S., Last, J. A., Murphy, C. J., and Pandit, A. Characterizing nanoscale topography of the aortic heart valve basement membrane for tissue engineering heart valve scaffold design. Tissue Eng. 2006; 12:413-421.
111. Yamasaki, Y., Makino, H., and Ota, Z. Meshwork structures in bovine glomerular and tubular basement membrane as revealed by ultra-high-resolution scanning electron microscopy. Nephron. 1994; 66:189-199.
112. Kawabe, T. T., MacCallum, D. K., and Lillie, J. H. Variation in basement membrane topography in human thick skin. Anat Rec. 1985; 211:142-148.
113. Ruben, G. C. and Yurchenco, P. D. High resolution platinum-carbon replication of freeze-dried basement membrane. Microsc Res Tech. 5-1-1994; 28:13-28.
114. Wojciak-Stothard, B., Curtis, A., Monaghan, W., MacDonald, K., and Wilkinson, C. Guidance and activation of murine macrophages by nanometric scale topography. Exp Cell Res. 3-15-1996; 223:426-435.
115. Wojciak-Stothard, B., Curtis, A. S., Monaghan, W., McGrath, M., Sommer, I., and Wilkinson, C. D. Role of the cytoskeleton in the reaction of fibroblasts to multiple grooved substrata. Cell Motil Cytoskeleton. 1995; 31:147-158.
116. Martin, J. Y., Schwartz, Z., Hummert, T. W., Schraub, D. M., Simpson, J., Lankford, J., Jr., Dean, D. D., Cochran, D. L., and Boyan, B. D. Effect of titanium surface roughness on proliferation, differentiation, and protein synthesis of human osteoblast-like cells (MG63). J Biomed Mater Res. 1995; 29:389-401.
117. Hohn, H. P., Steih, U., and Denker, H. W. A novel artificial substrate for cell culture: effects of substrate flexibility/malleability on cell growth and morphology. In Vitro Cell Dev Biol Anim. 1995; 31:37-44.
118. Fitton, J. H., Dalton, B. A., Beumer, G., Johnson, G., Griesser, H. J., and Steele, J. G. Surface topography can interfere with epithelial tissue migration. J Biomed Mater Res. 1998; 42:245-257.
119. Steele, J. G., Johnson, G., McLean, K. M., Beumer, G. J., and Griesser, H. J. Effect of porosity and surface hydrophilicity on migration of epithelial tissue over synthetic polymer. J Biomed Mater Res. 6-15-2000; 50:475-482.
120. Lee, J. H., Lee, S. J., Khang, G., and Lee, H. B. Interaction of fibroblasts on polycarbonate membrane surfaces with different micropore sizes and hydrophilicity. J Biomater Sci Polym Ed. 1999; 10:283-294.
121. Karuri, N. W., Porri, T. J., Albrecht, R. M., Murphy, C. J., and Nealey, P. F. Nano- and microscale holes modulate cell-substrate adhesion, cytoskeletal organization, and -beta1 integrin localization in SV40 human corneal epithelial cells. IEEE Trans Nanobioscience. 2006; 5:273-280.
122. Teixeira, A. I., Abrams, G. A., Bertics, P. J., Murphy, C. J., and Nealey, P. F. Epithelial contact guidance on well-defined micro- and nanostructured substrates. J Cell Sci. 5-15-2003; 116:1881-1892.
123. Curtis, A. S. and Clark P. The effect of topographic and mechanical properties on materials on cell behavior. Crit Rev Biocompatability. 1990; 5:343-362.
124. Chen, C. S., Mrksich, M., Huang, S., Whitesides, G. M., and Ingber, D. E. Geometric control of cell life and death. Science. 5-30-1997; 276:1425-1428.
125. Ruoslahti, E. Stretching is good for a cell. Science. 5-30-1997; 276:1345-1346.
126. Chen, D., Magnuson, V., Hill, S., Arnaud, C., Steffensen, B., and Klebe, R. J. Regulation of integrin gene expression by substrate adherence. J Biol Chem. 11-25-1992; 267:23502-23506.
127. Hormia, M. and Kononen, M. Immunolocalization of fibronectin and vitronectin receptors in human gingival fibroblasts spreading on titanium surfaces. J Periodontal Res. 1994; 29:146-152.
128. Ertel, S. I., Ratner, B. D., Kaul, A., Schway, M. B., and Horbett, T. A. In vitro study of the intrinsic toxicity of synthetic surfaces to cells. J Biomed Mater Res. 1994; 28:667-675.
129. Waldegger, S. and Lang, F. Cell volume and gene expression. J Member Biol. 3-15-1998; 162:95-100.
130. Karuri, N. W., Liliensiek, S., Teixeira, A. I., Abrams, G., Campbell, S., Nealey, P. F., and Murphy, C. J. Biological length scale topography enhances cell-substratum adhesion of human corneal epithelial cells. J Cell Sci. 7-1-2004; 117:3153-3164.
131. Teixeira, A. I., Nealey, P. F., and Murphy, C. J. Responses of human keratocytes to micro- and nanostructured substrates. J Biomed Mater Res A. 12-1-2004; 71:369-376.
132. Liliensiek, S. J., Campbell, S., Nealey, P. F., and Murphy, C. J. The scale of substratum topographic features modulates proliferation of corneal epithelial cells and corneal fibroblasts. J Biomed Mater Res A. 2006; 79:185-192.
133. Whitesides, G. M., Ostuni, E., Takayama, S., Jiang, X., and Ingber, D. E. Soft lithography in biology and biochemistry. Annu Rev Biomed Eng 2001. 2003; 335-373.
134. Floyd, B. B., Cleveland, P. H., and Worthen, D. M. Fibronectin in human trabecular drainage channels. Invest Ophthalmol Vis Sci. 1985; 26:797-804.
135. Clark, A. F., Wilson, K., de Kater, A. W., Allingham, R. R., and McCartney, M. D. Dexamethasone-induced ocular hypertension in perfusion-cultured human eyes. Invest Ophthalmol Vis Sci. 1995; 36:478-489.
136. Oh, D. J., Martin, J. L., Williams, A. J., Peck, R. E., Pokorny, C., Russell, P., Birk, D. E., and Rhee, D. J. Analysis of expression of matrix metalloproteinases and tissue inhibitors of metalloproteinases in human ciliary body after latanoprost. Invest Ophthalmol Vis Sci. 2006; 47:953-963.
137. Hengsberger, S., Kulik, A., and Zysset, P. A combined atomic force microscopy and nanoindentation technique to investigate the elastic properties of bone structural units. Eur Cell Mater. 1-10-2001; 1:12-17.
138. Hansma, H. G., Clegg, D. O., Kokkoli, E., Oroudjev, E., and Tirrell, M. Analysis of matrix dynamics by atomic force microscopy. Methods Cell Biol. 2002; 69:163-193.
139. Rhee, D. J., Tamm, E. R., and Russell, P. Donor corneoscleral buttons: a new source of trabecular meshwork for research. Exp Eye Res. 2003; 77:749-756.
140. Teixeira, A. I., McKie, G. A., Foley, J. D., Bertics, P. J., Nealey, P. F., and Murphy, C. J. The effect of environmental factors on the response of human corneal epithelial cells to nanoscale substrate topography. Biomaterials. 2006; 27:3945-3954.
141. Tumminia, S. J. and Russell, P. Alpha B-crystallin accumulation in human astroglioma cell line U373MG is stress-dependent and phosphorylation-independent. J Biochem (Tokyo). 1994; 116:973-979.

It is understood that the examples and embodiments described herein are for illustrative purposes only and that various modifications or changes in light thereof will be suggested to persons skilled in the art and are to be included within the spirit and purview of this application and scope of the appended claims. All publications, sequences represented by accession numbers, patents, and patent applications cited herein are hereby incorporated by reference in their entirety for all purposes.

1. A method of identifying an agent that modulates the intrinsic compliance of a trabecular meshwork (TM) cell, comprising:
a) contacting the agent to a test TM cell cultured in vitro;
b) determining the compliance of the test TM cell, wherein a Young's modulus of the test TM cell that is different from the Young's modulus of a control TM cell not contacted with the agent indicates that the agent modulates the compliance of the TM cell, thereby identifying an agent that modulates the intrinsic compliance of the (TM) cell.

2. The method of claim 1, wherein the intrinsic compliance of a single TM cell or a region of a TM cell is measured.

3. The method of claim 1, wherein the TM cell is cultured on a surface having fabricated topographical features.

4. The method of claim 1, wherein the TM cell is cultured on a surface having different values of compliance.

5. The method of claim 1, wherein the TM cell is cultured on a surface with a Young's modulus value in the range of about 3 kPa to about 100 kPa.

6. The method of claim 1, wherein the TM cell is cultured on a hydrogel.

7. The method of claim 1, wherein the TM cell is from a glaucomatous subject.

8. The method of claim 1, wherein the TM cell is from a non-glaucomatous subject.

9. The method of claim 1, wherein the intrinsic compliance is measured by a method selected from the group consisting of indenting, pulling, compressing, shearing, bending, buckling, wave propagation, and drilling.

10. The method of claim 1, wherein the intrinsic compliance is measured by atomic force microscopy (ATM).

11. The method of claim 1, wherein the TM cell is from the juxtacanalicular region (JCT).

12. The method of claim 1, wherein the agent increases the intrinsic compliance of the TM cell.

13. The method of claim 1, wherein the agent disrupts the cytoskeleton of the TM cell.

14. The method of claim 1, wherein the agent decreases the intrinsic compliance of the TM cell.

15. An agent that modulates the intrinsic compliance of a trabecular meshwork (TM) cell, wherein the agent is identified according to the method of claim 1.

16. A method of identifying an agent that modulates the intrinsic compliance of an extracellular matrix (ECM) within a trabecular meshwork (TM), comprising:
a) contacting the agent to a test ECM of TM tissue cultured in vitro;
b) determining the compliance of the test ECM, wherein a Young's modulus of the test ECM that is different from the Young's modulus of a control ECM not contacted with the agent indicates that the agent modulates the compliance of the ECM, thereby identifying an agent that modulates the intrinsic compliance of the ECM.

17-28. (canceled)

29. An agent that modulates the intrinsic compliance of an extracellular matrix (ECM) within a trabecular meshwork (TM), wherein the agent is identified according to the method of claim 16.

30. A method of increasing the compliance of a trabecular meshwork (TM), comprising contacting the TM with an agent that increases the compliance of a TM cell identified by the method of claim 1, thereby increasing the compliance of the TM.

31-32. (canceled)

33. A method of reducing intraocular pressure in a subject in need thereof, comprising administering to the subject an effective amount of an agent that increases the compliance of a TM cell and/or an agent that increases the compliance of an ECM in the TM, wherein the outflow of aqueous humor through the TM is increased, thereby reducing intraocular pressure in the subject.

34-38. (canceled)

39. A method of reducing the severity of glaucoma in a subject in need thereof, comprising administering to the subject an effective amount of an agent that increases the compliance of the TM cell and/or an agent that increases the compliance of the ECM in the TM, wherein the outflow of aqueous humor through the TM is increased, thereby reducing the severity of glaucoma in the subject.

40-43. (canceled)
